(19) United States
(12) Patent Application Publication
(10) Pub. No.: US 2024/0216222 A1
(43) Pub. Date: Jul. 4, 2024

(54) CLOSED SYSTEM TRANSFER DEVICE

(71) Applicants: B. Braun Medical Inc., Bethlehem, PA (US); B. Braun Melsungen AG, Melsungen (DE)

(72) Inventors: Karl-Martin Berg, Melsungen (DE); Florin Kopp, Schortens (DE); Uwe Erik Schneider, Spangenberg (DE); Varaprasad Sikhile, Hamburg (DE); Christian Walter, Kassel (DE); Bruce W. Brunetti, Phillipsburg, NJ (US); Scott Alan Moyer, Green Lane, PA (US); Gary Higgins, Waco, TX (US); James Albert Nixon, Allentown, PA (US); Nicholas Panick, Irvine, CA (US)

(21) Appl. No.: 18/557,847

(22) PCT Filed: Apr. 28, 2022
(86) PCT No.: PCT/US2022/026752

§ 371 Date: Oct. 27, 2023

Related U.S. Application Data
(60) Provisional application No. 63/181,313, filed on Apr. 29, 2021.
(60) Provisional application No. 63/181,387, filed on Apr. 29, 2021.
(60) Provisional application No. 63/181,429, filed on Apr. 29, 2021.
(60) Provisional application No. 63/181,446, filed on Apr. 29, 2021.
(60) Provisional application No. 63/181,457, filed on Apr. 29, 2021.
(60) Provisional application No. 63/196,735, filed on Jun. 4, 2021.

(51) Int. Cl. A61J 1/20; A61J 1/14

(52) U.S. Cl. CPC A61J 1/2051; A61J 1/1406; A61J 1/201; A61J 1/2068; A61J 1/2082; A61J 1/2096

(57) ABSTRACT

A closed system transfer device includes a first adaptor and a second adaptor that attach with one another in a coupled state to form a sealed fluid passage between two reservoirs. The first adaptor can be attached to a first reservoir, and the second adaptor can be attached to a second reservoir. One or both adaptors can include a septum that engages another septum during coupling and decoupling to prevent liquid or vapor from being released to the environment. The device can also include a vent line that equalizes pressure between the two reservoirs. The vent line can include at least one filter to filter gas in device. In addition, or in the alternative, the vent line can be connected in fluid communication with an inflatable membrane that stores gas inside the device and assists in equalizing pressure.

### CROSS-REFERENCES TO RELATED APPLICATIONS

This application claims priority to U.S. Provisional Application No. 63/181,313, filed Apr. 29, 2021, U.S. Provisional Application No. 63/181,387, filed Apr. 29, 2021, U.S. Provisional Application No. 63/181,429, filed Apr. 29, 2021, U.S. Provisional Application No. 63/181,446, filed Apr. 29, 2021, U.S. Provisional Application No. 63/181,457, filed Apr. 29, 2021, and U.S. Provisional Application No. 63/196,735, filed Jun. 4, 2021. The contents of all of the foregoing applications are incorporated by reference herein in their entireties and for all purposes.

### FIELD

The present disclosure relates to devices for safely transferring hazardous drugs between containers in a closed system that prevents the inflow of contaminants into the system, and prevents the release of hazardous vapors from the system into the environment.

### BACKGROUND

Closed system transfer devices (CSTDs) are systems used to transfer medication from one reservoir or vessel (e.g. a syringe) to another reservoir or vessel (e.g. a vial), while limiting the potential for drug aerosolization, drug contamination, sharps exposure, and hazardous drug exposure. Once connected between reservoirs, CSTD devices can equalize pressure gradients between the reservoirs. Without a pressure equalization system, differences in pressure can lead to the generation of fine aerosols that escape into the air and expose the environment, patients, and health care professionals to hazardous drugs.

### SUMMARY

A closed system transfer device includes a first adaptor and a second adaptor that attach with one another in a coupled state to form a sealed fluid passage between two reservoirs.

In one aspect of the disclosure, a closed system transfer device includes a first adaptor that can attach to a first reservoir. The first adaptor can define a first passage and have a first septum that seals an end of the first passage. The device can also have a second adaptor configured to attach to a second reservoir. The second adaptor can include a housing having an interior and defining a second passage. A second septum can seal an end of the second passage.

In another aspect of the disclosure, the device can include a carrier that is movable in the interior of the second adaptor. The carrier can define a chamber that contains at least a portion of the second septum. A needle with a needle opening can be disposed in the interior of the second adaptor.

In another aspect of the disclosure, the interior of the second adaptor can be adapted to receive the first adaptor in a telescoping manner, with the first adaptor insertable into the second adaptor.

In another aspect of the disclosure, the carrier can be displaceable in the interior of the second adaptor, and relative to the needle, by the first adaptor when the first adaptor is inserted into the second adaptor.

In another aspect of the disclosure, the carrier can be displaceable within the second adaptor between a first position, in which the first septum abuts the second septum and the needle opening is sealed inside the second passage, and a second position, in which the first septum abuts the second septum and the needle opening is in fluid communication with the first passage to connect the first adaptor and the second adaptor in a fluid path open state.

In another aspect of the disclosure, the device can include a releasable lock that locks the first adaptor inside the second adaptor after the first adaptor is inserted into the second adaptor.

In another aspect of the disclosure, the releasable lock can lock the first adaptor inside the second adaptor when the carrier is displaced to the second position.

In another aspect of the disclosure, the releasable lock can include a first locking element on the carrier and a second locking element in the housing.

In another aspect of the disclosure, the releasable lock can include a first locking element on the first adaptor and a second locking element on the second adaptor.

In another aspect of the disclosure, the releasable lock can be released by pressing at least one side of the housing radially inwardly.

In another aspect of the disclosure, at least one side of the housing can include at least one push button.

In another aspect of the disclosure, the at least one push button can be depressible radially inwardly to disengage a portion of the carrier from a section of the housing.

In another aspect of the disclosure, the portion of the carrier can include at least one locking lug and the section of the housing can include at least one locking ramp inside the housing.

In another aspect of the disclosure, the at least one push button can be depressible radially inwardly to disengage a portion of the first adaptor from a section of the second adaptor.

In another aspect of the disclosure, the portion of the first adaptor can include at least one flange and the section of the second adaptor can include at least one retaining clip attached to the at least one push button.

In another aspect of the disclosure, the releasable lock can be released by rotating the second adaptor relative to the first adaptor.

In another aspect of the disclosure, the second adaptor can include a third septum that is axially spaced from the second septum.

In another aspect of the disclosure, the needle opening can be sealed between the second septum and the third septum when the carrier is in the first position.

In another aspect of the disclosure, the second septum can include a piston having a piston head at least partially contained in the carrier and a collapsible midsection.

In another aspect of the disclosure, the collapsible midsection of the piston can define a hollow core, and the needle can be at least partially contained inside the hollow core.

In another aspect of the disclosure, the device can include a female Luer connector rotatably mounted to the housing of the second adaptor.

In another aspect of the disclosure, the female Luer connector can include a thread that can mate with a threaded connection on the second reservoir.

In another aspect of the disclosure, the first adaptor can include a male Luer connector.

In another aspect of the disclosure, the first adaptor can include a vial spike.

In another aspect of the disclosure, the first adaptor and the second adaptor can be rectangular.

In another aspect of the disclosure, the first adaptor and the second adaptor can be cylindrical.

In another aspect of the disclosure, the needle can be fixed in the interior of the second adaptor.

In another aspect of the disclosure, the releasable lock can include a first locking element on the carrier and a second locking element on the first adaptor.

In another aspect of the disclosure, a section of the carrier can include at least one locking aperture and a portion of the housing can include at least one detent.

In another aspect of the disclosure, the releasable lock can include a locking arm extending through a slot in a wall of the housing.

In another aspect of the disclosure, the locking arm can be pivotally mounted in the slot on at least one hinge.

In another aspect of the disclosure, the locking arm can be pivotally mounted in the slot between a locking position that locks the first adaptor inside the second adaptor and a release position that permits the first adaptor to be removed from the second adaptor.

In another aspect of the disclosure, the locking arm can include a first end that projects radially outwardly from the wall of the housing when the locking arm is in the locking position.

In another aspect of the disclosure, the first end of the locking arm can include a button.

In another aspect of the disclosure, the locking arm can include a second end that projects radially inwardly from the wall of the housing when the locking arm is in the locking position.

In another aspect of the disclosure, the second end can include a detent that engages the carrier when the locking arm is in the locking position.

In another aspect of the disclosure, the detent can include a ramped surface and an abutment surface.

In another aspect of the disclosure, the carrier can include a locking aperture adapted to receive the detent when the carrier is in the second position and when the locking arm is in the locking position.

In another aspect of the disclosure, the locking aperture can include an abutment edge that engages the abutment surface of the detent when the carrier is in the second position and when the locking arm is in the locking position to prevent displacement of the carrier out of the second position.

In another aspect of the disclosure, the second adaptor can include a third septum that is axially spaced from the second septum.

In another aspect of the disclosure, the needle opening can be sealed between the second septum and the third septum when the carrier is in the first position.

In another aspect of the disclosure, a closed system transfer device includes a first adaptor configured to attach to a first reservoir. The first adaptor can define a first passage and have a first septum that seals an end of the first passage. The device can also have a second adaptor configured to attach to a second reservoir. The second adaptor can include a housing having an interior and defining a second passage. A second septum can seal an end of the second passage.

In another aspect of the disclosure, the device can include a carrier that is movable in the interior of the second adaptor. The carrier can define a chamber that contains at least a portion of the second septum. A needle with a needle opening can be fixed in the interior of the second adaptor.

In another aspect of the disclosure, the interior of the first adaptor can be adapted to receive the second adaptor in a telescoping manner.

In another aspect of the disclosure, the carrier can be displaceable in the interior of the second adaptor, and relative to the needle, by an inner portion of the first adaptor when the second adaptor is inserted into the first adaptor.

In another aspect of the disclosure, the carrier can be displaceable within the second adaptor between a first position, in which the first septum abuts the second septum and the needle opening is sealed inside the second passage, and a second position, in which the first septum abuts the second septum and the needle opening is in fluid communication with the first passage to connect the first adaptor and the second adaptor in a fluid path open state.

In another aspect of the disclosure, the device can include a releasable lock that locks the second adaptor inside the first adaptor after the second adaptor is inserted into the first adaptor.

In another aspect of the disclosure, the releasable lock can lock the second adaptor inside the first adaptor when the carrier is displaced to the second position.

In another aspect of the disclosure, the releasable lock can include a first locking element on the first adaptor and a second locking element on the second adaptor.

In another aspect of the disclosure, the releasable lock can be released by pressing a side of the first adaptor radially inwardly.

In another aspect of the disclosure, the side of the first adaptor can include a push button.

In another aspect of the disclosure, the push button can be depressible radially inwardly to disengage a portion of the first adaptor from a section of the housing.

In another aspect of the disclosure, the portion of the first adaptor can include at least one locking aperture and the section of the housing can include at least one locking ramp that extends radially outwardly from the housing.

In another aspect of the disclosure, the at least one locking ramp can include a leading end, a trailing end and a ramped surface between the leading end and trailing end.

In another aspect of the disclosure, an abutment surface in the at least one locking aperture can engage the trailing end of the at least one locking ramp to lock the second adaptor to the first adaptor.

In another aspect of the disclosure, the ramped surface can include a straight section that is adjacent to the leading end and a curved section that extends between the straight section and the trailing end.

In another aspect of the disclosure, the curved section can have a compound curvature that defines a concave portion and a convex portion.

In another aspect of the disclosure, the releasable lock can include a locking arm extending through a slot in a wall of the first adaptor.

In another aspect of the disclosure, the locking arm can be pivotally mounted in the slot on at least one hinge.

In another aspect of the disclosure, the locking arm can be pivotally mounted in the slot between a locking position that locks the second adaptor inside the first adaptor and a release position that permits the second adaptor to be removed from the first adaptor.

In another aspect of the disclosure, the locking arm can include a first end that projects radially outwardly from the wall of the housing when the locking arm is in the locking position.

In another aspect of the disclosure, the first end can include a button.

In another aspect of the disclosure, the locking arm can include a second end positioned in the slot when the locking arm is in the locking position.

In another aspect of the disclosure, the second end can include an abutment surface that engages the housing when the locking arm is in the locking position.

In another aspect of the disclosure, the second adaptor can include a third septum that is axially spaced from the second septum.

In another aspect of the disclosure, the needle opening can be sealed between the second septum and the third septum when the carrier is in the first position.

In another aspect of the disclosure, the first adaptor can include a male Luer connector.

In another aspect of the disclosure, a vial spike can include a housing and a spike connector extending from the housing.

In another aspect of the disclosure, the housing and spike connector can define a vent line and a transfer line that is separate from the vent line.

In another aspect of the disclosure, the vent line can include a hydrophobic filter in the housing.

In another aspect of the disclosure, the vent line can include an activated carbon filter in series with the hydrophobic filter.

In another aspect of the disclosure, the housing can include a first housing portion having a dry break coupling fluidly connected with the transfer line.

In another aspect of the disclosure, the dry break coupling can include a mating element for connecting the vial spike to a first fluid reservoir.

In another aspect of the disclosure, the housing can include a second housing portion from which the spike connector extends.

In another aspect of the disclosure, the first housing portion can include a first cover piece and the second housing portion can include a second cover piece configured to connect with the first cover piece and form a narrow space therebetween.

In another aspect of the disclosure, the first cover piece can include a ring-shaped lip portion extending at least partially around a periphery of the first cover piece, and the second cover piece comprises a ring-shaped wall portion extending at least partially around a periphery of the second cover piece, the ring-shaped wall portion adapted to the receive ring-shaped lip portion to join the first housing portion to the second housing portion in a mated arrangement.

In another aspect of the disclosure, the ring-shaped lip portion of the first cover piece can include a divider wall that extends into the narrow space formed by the first cover piece and second cover piece.

In another aspect of the disclosure, the divider wall can define a first chamber in the narrow space on a first side of the divider wall, and a second chamber in the narrow space on a second side of the divider wall.

In another aspect of the disclosure, the first chamber can fluidly connect with the vent line but not the transfer line, and the second chamber can fluidly connect with the transfer line but not the vent line.

In another aspect of the disclosure, the hydrophobic filter can be housed in the first chamber.

In another aspect of the disclosure, the spike connector can define a first passage fluidly connected to the first chamber but not the second chamber, and a second passage fluidly connected to the second chamber but not the first chamber.

In another aspect of the disclosure, the first passage can extend parallel to the second passage in the spike connector.

In another aspect of the disclosure, the transfer line can include a particle filter.

In another aspect of the disclosure, the particle filter can be housed in the second chamber arranged parallel to the hydrophobic filter.

In another aspect of the disclosure, the housing can include a third housing portion that houses the activated carbon filter, and the vent line can pass from the first housing portion into the third housing portion and exit to the atmosphere through an outlet formed through a wall of the third housing portion.

In another aspect of the disclosure, the vent line can include a check valve.

In another aspect of the disclosure, the housing can include a first housing portion having a dry break coupling fluidly connected with the transfer line.

In another aspect of the disclosure, the dry break coupling can include a mating element for connecting the vial spike to a first fluid reservoir.

In another aspect of the disclosure, the housing can include a second housing portion from which the spike connector extends.

In another aspect of the disclosure, the first housing portion can include a first cover piece and the second housing portion can include a second cover piece configured to connect with the first cover piece and form a narrow space therebetween.

In another aspect of the disclosure, the first cover piece can include a ring-shaped lip portion extending at least partially around a periphery of the first cover piece, and the second cover piece can include a ring-shaped wall portion extending at least partially around a periphery of the second cover piece, the ring-shaped wall portion adapted to the receive ring-shaped lip portion to join the first housing portion to the second housing portion in a mated arrangement.

In another aspect of the disclosure, the ring-shaped lip portion of the first cover piece can include a divider wall that extends into the narrow space formed by the first cover piece and second cover piece.

In another aspect of the disclosure, the divider wall can define a first chamber in the narrow space on a first side of the divider wall, and a second chamber in the narrow space on a second side of the divider wall.

In another aspect of the disclosure, the first chamber can fluidly connect with the vent line but not the transfer line, and the second chamber can fluidly connect with the transfer line but not the vent line.

In another aspect of the disclosure, the hydrophobic filter can be housed in the first chamber.

In another aspect of the disclosure, the spike connector can define a first passage fluidly connected to the first chamber but not the second chamber, and a second passage fluidly connected to the second chamber but not the first chamber.

In another aspect of the disclosure, the first passage can extend parallel to the second passage in the spike connector.

In another aspect of the disclosure, the transfer line can include a particle filter.

In another aspect of the disclosure, the particle filter can be housed in the second chamber arranged parallel to the hydrophobic filter.

In another aspect of the disclosure, the housing can include a third housing portion in fluid communication with the vent line, and the third housing portion can be connected to a flexible membrane that forms a gas storage volume between the third housing portion and flexible membrane.

In another aspect of the disclosure, a vial adaptor can include a vial spike according to any one of the preceding aspect and a vial clip connectable to the vial spike.

In another aspect of the disclosure, the vial clip can include a proximal end with a fastener mechanism configured to connect to the vial spike.

In another aspect of the disclosure, the fastener mechanism can include a plurality of flexible arms, each flexible arm having a barbed end.

In another aspect of the disclosure, the flexible arms can releasably engage a portion of the housing of the vial spike to connect the vial clip to the vial spike.

In another aspect of the disclosure, the vial clip can include at least one arcuate flange forming a receptacle.

In another aspect of the disclosure, the spike connector can extend into the receptacle, and the at least one arcuate flange can form a guard to protect a user from accidentally being sticked by the spike connector.

### BRIEF DESCRIPTION OF THE DRAWINGS

The drawing figures depict one or more implementations by way of example only, not by way of limitations. In the figures, like reference numerals can refer to the same or similar elements.

FIG. 1 is a perspective view of a CSTD according to one embodiment.

FIG. 2 is an exploded perspective view of the CSTD of FIG. 1.

FIG. 3 is a front view of the CSTD of FIG. 1 shown in cross section, with components of the CSTD shown in an uncoupled state.

FIG. 4 is a front view of the CSTD of FIG. 1 shown in cross section, with components of the CSTD shown in a coupled and locked state.

FIG. 5 is a cross sectional view of the CSTD of FIG. 1 taken through a first section.

FIG. 6 is a cross sectional view of the CSTD of FIG. 1 taken through a second section.

FIG. 7 is a perspective view of a CSTD according to another embodiment.

FIG. 8 is an exploded perspective view of the CSTD of FIG. 7.

FIG. 9 is a front view of the CSTD of FIG. 7 shown in cross section, with components of the CSTD shown in an uncoupled state.

FIG. 10 is a front view of the CSTD of FIG. 7 shown in cross section, with components of the CSTD shown in a coupled and locked state.

FIG. 11 is a perspective view of a CSTD according to another embodiment.

FIG. 12 is an exploded perspective view of the CSTD of FIG. 11.

FIG. 13 is a front view of the CSTD of FIG. 11 shown in cross section, with components of the CSTD shown in an uncoupled state.

FIG. 14 is a front view of the CSTD of FIG. 11 shown in cross section, with components of the CSTD shown in a coupled and locked state.

FIG. 15 is a cross sectional view of the CSTD of FIG. 11 taken through a section of a housing.

FIG. 16 is a truncated cross sectional view of a CSTD according to another embodiment.

FIG. 17 is a perspective view of a CSTD according to another embodiment.

FIG. 18 is a perspective view of the CSTD of FIG. 17, with components of the CSTD shown in an uncoupled state.

FIG. 19 is an exploded perspective view of the CSTD of FIG. 17.

FIG. 20 is a front view of the CSTD of FIG. 17 shown in cross section, with components of the CSTD shown in a coupled and locked state.

FIG. 21 is a cross sectional view of the CSTD of FIG. 17 taken through a first section.

FIG. 22 is a cross sectional view of the CSTD of FIG. 17 taken through a second section.

FIG. 23 is a perspective view of a CSTD according to another embodiment.

FIG. 24 is a perspective view of the CSTD of FIG. 23, with components shown in an uncoupled state.

FIG. 25 is an exploded perspective view of the CSTD of FIG. 23.

FIG. 26 is a front view of the CSTD of FIG. 23 shown in cross section through a first plane, with components of the CSTD shown in an uncoupled state.

FIG. 27 is a side view of the CSTD of FIG. 23 shown in cross section through a second plane, with components of the CSTD shown in an uncoupled state.

FIG. 28 is a front view of the CSTD of FIG. 23 shown in cross section through the first plane, with components of the CSTD shown in a coupled and locked state.

FIG. 29 is a side view of the CSTD of FIG. 23 shown in cross section through the second plane, with components of the CSTD shown in a coupled and locked state.

FIG. 30 is a cross sectional view of the CSTD of FIG. 23 through a third plane perpendicular to the first plane and second plane, with components of the CSTD shown in a coupled and locked state.

FIG. 31 is a perspective view of a CSTD according to another embodiment.

FIG. 32 is a perspective view of the CSTD of FIG. 31, with components shown in an uncoupled state.

FIG. 33 is an exploded perspective view of the CSTD of FIG. 31.

FIG. 34 is a front view of the CSTD of FIG. 31, shown in cross section through a first plane, with components shown in the uncoupled state.

FIG. 35 is a front view of the CSTD of FIG. 31, shown in cross section through the first plane, with components shown in the coupled and locked state.

FIG. 36 is a cross section view of the CSTD of FIG. 31 taken through a second plane perpendicular to the first plane, with components shown in the coupled and locked state.

FIG. 37 is a front view of a CSTD according to another embodiment, showing a vial spike and vial clip in a coupled state, with a portion of the vial spike broken away to illustrate an interior component.

FIG. 38 is a perspective view of the CSTD of FIG. 37, showing the vial spike and vial clip in an uncoupled state.

FIG. 39 is a front view of the vial spike of FIG. 37.

FIG. 40 is an exploded perspective view of the vial spike of FIG. 37.

FIG. 41 is a front view of the vial spike of FIG. 37 shown in cross section.

FIG. 42 is a bottom view of a housing portion of the vial spike of FIG. 37.

FIG. 43 is a side view of the vial spike of FIG. 37 shown in cross section.

FIG. 44 is a perspective view of the vial spike of FIG. 37 with a housing portion shown transparent to illustrate flow directions inside the vial spike.

FIG. 45 is a magnified cross section view of another embodiment of a vial spike, showing an alternate arrangement.

FIG. 46 is a front view of a CSTD according to another embodiment, showing an alternate vial spike and vial clip in a coupled state.

FIG. 47 is a perspective view of a CSTD according to another embodiment, showing a vial spike and vial clip in a coupled state.

FIG. 48 is a front view of the CSTD of FIG. 47.

FIG. 49 is an exploded front view of the CSTD of FIG. 47.

FIG. 50 is a first cross sectional view of the vial spike of FIG. 47.

FIG. 51 is a second cross sectional view of the vial spike of FIG. 47.

FIG. 52 is a perspective view of a modular system for assembling CSTDs according to another embodiment, with components shown in a disassembled state.

FIG. 53 is an exploded perspective view of some of the components of the modular system shown in FIG. 52.

FIG. 54 is a cross sectional view of components of the modular system shown in FIG. 52.

FIG. 55 is a top view of one of the components shown in FIG. 52.

FIG. 56 is an enlarged perspective view of another component shown in FIG. 52.

FIG. 57 is a top view of some of the components in FIG. 52 shown in partial cross section.

FIG. 58 is a front view of a CSTD that can be assembled from components in the modular system of FIG. 52.

FIG. 59 is a front view of another CSTD that can be assembled from components in the modular system of FIG. 52.

FIG. 60 is a front view of another CSTD that can be assembled from components in the modular system of FIG. 52.

FIG. 61 is a front view of another CSTD that can be assembled from components in the modular system of FIG. 52.

FIG. 62 is a perspective view of a modular system for assembling CSTDs according to another embodiment, with components shown in a disassembled state.

FIG. 63 is an exploded perspective view of some of the components shown in FIG. 62 that can be assembled to form a CSTD.

FIG. 64 is a top view of some of the components shown in FIG. 63, the components being shown in an assembled state and in partial cross section.

FIG. 65 is an exploded perspective view of other components shown in FIG. 62 that can be assembled to form another CSTD.

FIG. 66 is a top view of some components shown in FIG. 65, the components being shown in an assembled state and in partial cross section.

FIG. 67 is a perspective view of a CSTD according to another embodiment.

FIG. 68 is a front view of the CSTD of FIG. 67, shown in cross section through a first plane, with components of the CSTD shown in a coupled state.

FIG. 69 is a side view of the CSTD of FIG. 67, shown in cross section through a second plane, with components of the CSTD shown in a coupled state.

FIG. 70 is an exploded perspective view of the CSTD of FIG. 67.

FIG. 71 is a perspective view of a CSTD according to another embodiment.

FIG. 72 is a front view of the CSTD of FIG. 71, shown in cross section through a first plane, with components of the CSTD shown in a coupled state.

FIG. 73 is a side view of the CSTD of FIG. 71, shown in cross section through a second plane, with components of the CSTD shown in a coupled state.

FIG. 74 is an exploded perspective view of the CSTD of FIG. 71.

FIG. 75 is a perspective view of a CSTD according to another embodiment.

FIG. 76 is a side view of the CSTD of FIG. 75, shown in cross section through a first plane, with components of the CSTD shown in a coupled state.

FIG. 77 is a front view of the CSTD of FIG. 75, shown in cross section through a second plane, with components of the CSTD shown in a coupled state.

FIG. 78 is a top view of the CSTD of FIG. 75, shown in cross section through a third plane, with components of the CSTD shown in a coupled state.

FIG. 79 is an exploded perspective view of the CSTD of FIG. 75.

FIG. 80 is a perspective view of a CSTD according to another embodiment.

FIG. 81 is a side view of the CSTD of FIG. 80, shown in cross section through a first plane, with components of the CSTD shown in a coupled state.

FIG. 82 is a front view of the CSTD of FIG. 80, shown in cross section through a second plane, with components of the CSTD shown in a coupled state.

FIG. 83 is a top view of the CSTD of FIG. 80, shown in cross section through a third plane, with components of the CSTD shown in a coupled state.

FIG. 84 is an exploded perspective view of the CSTD of FIG. 80.

FIG. 85 is a perspective view of a CSTD according to another embodiment.

FIG. 86 is another perspective view of the CSTD of FIG. 85, with components of the CSTD shown in an uncoupled state.

FIG. 87 is an exploded perspective view of the CSTD of FIG. 85.

FIG. 88 is a front view of the CSTD of FIG. 85 shown in cross section, with components of the CSTD shown in the uncoupled state.

FIG. 89 is a front view of the CSTD of FIG. 85 shown in cross section, with components of the CSTD shown in a coupled and locked state.

FIG. 90 is a front view of a CSTD according to another embodiment, with components of the CSTD shown in an uncoupled state.

FIG. 91 is a front of view of the CSTD of FIG. 90, with components of the CSTD shown in a partially inserted state.

FIG. 92 is a front of view of the CSTD of FIG. 90, with components of the CSTD shown in a coupled and locked state.

FIG. 93 is a front view of a CSTD according to another embodiment, with components of the CSTD shown in an uncoupled state.

FIG. 94 is a front of view of the CSTD of FIG. 93, with components of the CSTD shown in a partially inserted state.

FIG. 95 is a front of view of the CSTD of FIG. 93, with components of the CSTD shown in a coupled and locked state.

### DETAILED DESCRIPTION

In the following detailed description, numerous specific details are set forth by way of examples in order to provide a thorough understanding of the relevant teachings. It will be understood that such examples are non-limiting. Numerous variations, changes, substitutions and combinations will occur to those skilled in the art without departing from the scope of the present disclosure and its teachings, and are part of the present disclosure. This includes a substitution of a feature shown in one example with a feature shown in another example, or a combination of a feature shown in one example with a feature shown in another example. All substitutions and combinations are considered part of this written description.

The following description uses various defined terms to describe the physical arrangement and/or orientation of individual parts. The term “longitudinal axis” means or refers to the central axis of an element extending through the long dimension of the element. The terms “axial” and “axially” mean or refer to the direction parallel to the longitudinal axis. The terms “radial” and “radially” mean or refer to the direction perpendicular to the longitudinal axis. The terms “inward” and “inwardly” when used with “radially” refer to a direction toward the longitudinal axis in the radial direction. The terms “outward” and “outwardly” when used with “radially” refer to a direction away the longitudinal axis in the radial direction.

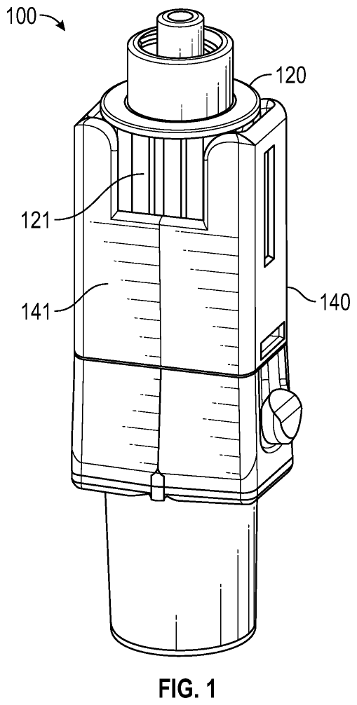

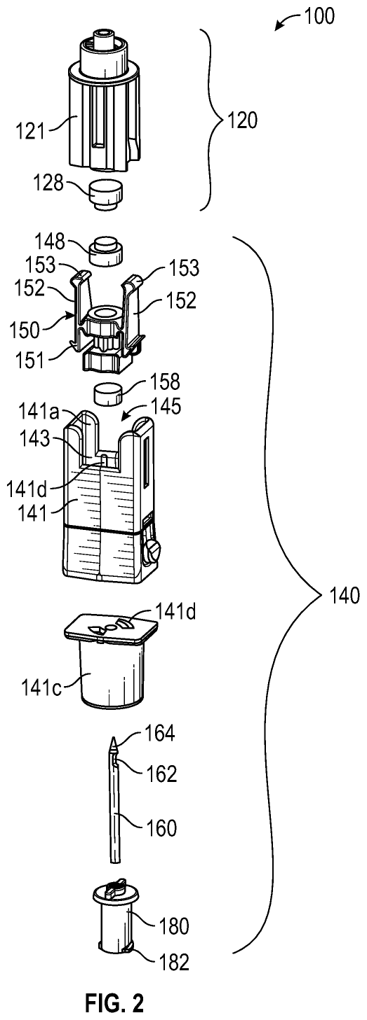

Referring to FIGS. 1 and 2, a CSTD 100 is shown according to a first embodiment. CSTD 100 has a first adaptor 120 configured to attach to a first fluid reservoir and a second adaptor 140 configured to attach to a second fluid reservoir. The first adaptor 120 can be connected to a vial, bag, or patient line, for example. The second adaptor 140 has a female Luer connector 180 that can be connected to a syringe or any male Luer connector on a fluid delivery device, such as a pump. Once first adaptor 120 and second adaptor 140 are attached to first and second fluid reservoirs, respectively, and interconnected to each other in the coupled state shown in FIG. 1, CSTD 100 forms a closed fluid passage between the first and second fluid reservoirs. The closed fluid passage is sealed from the outside environment, preventing the inflow of contaminants into the system and the release of hazardous vapors from the system.

First adaptor 120 has a generally rectangular body 121. Second adaptor 140 has a generally rectangular receptacle or housing 141. Housing 141 is adapted to receive body 121 of first adaptor 120 in a guided manner so that the two adaptors are axially aligned and centered during mating. Housing 141 has a hollow interior 143 and a socket 145 adapted to receive first adaptor 120 during mating. Two cut-outs extend on opposite sides of socket 145 as shown. The cut-outs provide greater access to the interior of housing 141, as compared to conventional adaptors. The greater access to the interior enables easier disinfection of both sides of the device.

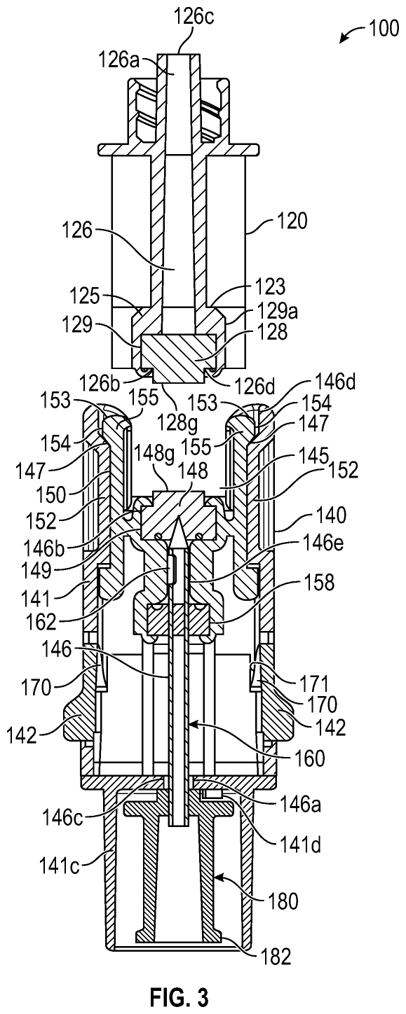

FIG. 3 shows a cross section of first adaptor 120 and second adaptor 140 prior to being coupled together. First adaptor 120 has a first passage 126 and a first septum 128 made of elastomeric material. First passage 126 has a first passage end 126a and a second passage end 126b opposite the first passage end. First passage end 126a defines a first opening 126c and second passage end 126b defines a second opening 126d. First passage 126 widens at the second passage end 126b to form a cylindrical chamber or seat 129. First septum 128 is received in seat 129 to seal the second passage end 126b. A portion of first septum 128 extends axially from second opening 126d, forming a first projection 128g.

Second adaptor 140 has a second passage 146 and a second septum 148 made of elastomeric material. Second passage 146 has a first passage end 146a and a second passage end 146b opposite the first passage end. First passage end 146a defines a first opening 146c and second passage end 146b defines a second opening 146d. First passage 146 widens at the second passage end 146b to form a cylindrical chamber or seat 149. Second septum 148 is received in seat 149 to seal the second passage end 146b. A portion of second septum 148 extends axially from seat 149, forming a second projection 148g. Second projection 148g is configured to abut with and be deformed by first projection 128g, and vice versa, to form a dry break coupling. A “dry break coupling”, as used herein, means a coupling that prevents liquid or vapor from being released from a CSTD during coupling or decoupling of two components.

Second septum 148 is contained in a carrier 150 that carries the second septum. Carrier 150 is configured to slide axially inside housing 141 between a first position and a second position. FIG. 3 shows carrier 150 in the first position. Carrier 150 has two flexible clips 152 that are made of a resilient flexible material. In the relaxed state, clips 152 extend outwardly in a parallel arrangement. Clips according to the present disclosure can also extend in a non-parallel arrangement in the relaxed state, however. For example, clips can also flare slightly outwardly and away from one another toward their free ends. This would allow the clips to store more energy when flexed inwardly, as will be explained.

Each clip 152 has a clip end 153 with a rounded projection 154 that extends radially outwardly from the clip and a pointed tip 155 that extends radially inwardly from the clip. Each rounded projection 154 rests against a tapered landing 147 formed in housing 141 when the carrier is in the first position. Clips 152 engage landings 147 to maintain the carrier in the first position, with the second septum located adjacent socket 145 of housing 141. FIG. 3 shows carrier 150 in the first position, with clips 152 in an outward relaxed position in engagement with landings 147.

First adaptor 120 is connected to second adaptor 140 by inserting body 121 into socket 145 and hollow interior 143 of second adaptor 140 until first septum 128 contacts second septum 148. Once first septum 128 abuts second septum 148, movement of carrier 150 out of the first position is resisted by the engagement between clips 152 and landings 147. This resistance is overcome by applying manual force to first adaptor 120 in the axial direction toward second septum 148 until the manual force exceeds the resistance force.

Seat 129 in first adaptor 120 has an outer wall 129a. Outer wall 129a temporarily engages and slides against the pointed tips 155 of the clips 152 as first adaptor 120 moves through socket 145 into hollow interior 143. This maintains the clips 152 in their resting positions against landings 147 and prevents the clips from flexing inwardly. Outer wall 129 tapers inwardly at a transition 123. In this arrangement, first adaptor 120 is insertable into second adaptor 140 so that outer wall 129a passes between clips 152 and slides through pointed tips 155.

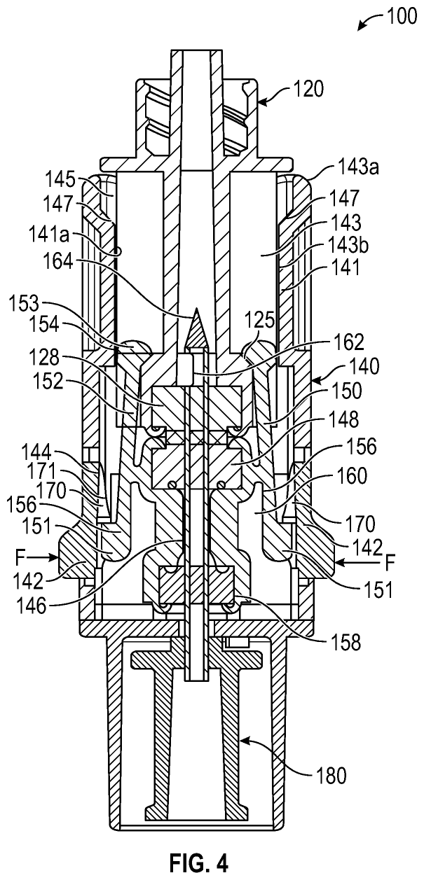

Once outer wall 129a passes through pointed tips 155, the pointed tips slide over the outer wall and reach transition 123. At this position, outer wall 129a no longer prevents inward flexion of clips 152, as the narrower dimension at transition 123 provides clearance to allow the clips to flex inward. Hollow interior 143 of second adaptor 140 narrows as it extends inwardly from the landings 147. Therefore, further advancement of first adaptor 120 into second adaptor 140 causes clip ends 153 to flex inwardly so that they no longer bear against landings 147. At this position, carrier 150 is free to move from the first position in housing 141 toward second position. FIG. 4 shows first adaptor 120 inserted fully into second adaptor 140, with carrier 150 moved to the second position and clips 152 flexed inwardly.

Hollow interior 143 of second adaptor 140 has a wider section 143a at socket 145, which is shown above landings 147 in FIG. 4. Hollow interior 143 transitions to a narrower section 143b beneath the landings. As carrier 150 is pushed from the first position toward the second position, the rounded projections 154 on clips 152 slide and bear against the inner wall 141a of housing 141. This abutment between rounded projections 154 and inner wall 141a causes clips 152 to flex inwardly as they enter narrower section 143b of hollow interior 143. The pointed tips 155 of clips 152 are pushed inwardly and rest against ledge portions 125 on outer wall 129a of first adaptor 120.

Second adaptor 140 houses a needle 160, as shown in FIGS. 3 and 4. Needle 160 is axially fixed to female Luer connector 180. Female Luer connector 180 is axially movable relative to housing 141 through a small axial distance, but is restricted from moving beyond a small range of motion. In this arrangement, needle 160 is captively held in housing 141 of second adaptor 140, and only permitted to move a small axial distance relative to the housing before being stopped from further axial movement. In contrast, carrier 150 and second septum 148 are axially movable relative to housing 141 through most of the length of hollow interior 143.

A variety of mechanisms can be used to limit axial movement of female Luer connectors and needles relative to housings. Examples include snap fit arrangements, such as those shown in U.S. Pat. Nos. 5,328,474 and 7,857,805, the contents of which are incorporated by reference herein in their entireties. FIG. 16 shows a modified female Luer connector 180′ with a snap fit arrangement that utilizes a barbed extension 182′. Barbed extension 182′ projects through an interior wall 142′ of a housing 141′ and into the hollow interior of the housing. Housing 141′ has collar ring 143′ with an annular detent 145′ in its interior. Barbed extension 182′ has a tapered leading end 183′ with a widened outer diameter. Annular detent 145′ forms a constricted passage 146′ inside collar ring 143′ that has a diameter smaller than the outer diameter of leading end 183′. In this arrangement, barbed extension 182′ can be inserted through interior wall 142′ and collar ring 143′ in a force fit matter to attach female Luer connector 180′ to housing 141′. Annular detent 145′ has a tapered face facing interior wall 142′ as shown which deflects or deforms a small amount to permit leading end 183′ to pass through the detent and emerge from collar ring 143′. A flange portion 147′ of female Luer connector 180′ abuts interior wall 142′ of housing 141′ on the side opposite collar ring 143′ to limit further axial displacement of the female Luer connector and needle 160′ into the housing. If female Luer connector 180′ is displaced in a direction opposite the direction of insertion, leading end 183′ reenters collar ring 143′ but abuts annular detent 145′. This abutment prevents female Luer connector 180′ from being reversed or pulled out of housing 141′. As a result, female Luer connector 180′ and needle 160′ are maintained in a captive arrangement in interior wall 142′ of housing 141′.

First septum 128 and second septum 148 are formed of elastomeric material that can be pierced by needle 160 as carrier 150 is pushed toward the second position. Needle 160 has a side opening 162 that remains sealed inside carrier 150 prior to coupling the second adaptor 140 to the first adaptor 120. Side opening 162 is sealed inside a narrow section 146e of second passage 146. Narrow section 146e is sealed at a first end by second septum 148 and sealed at a second end by a third septum 158.

As first adaptor 120 is advanced into housing 141, first septum 128 pushes against second septum 148 and moves the second septum and carrier 150 downwardly in the housing. Second septum 148 and carrier 150 are moved downwardly over needle 160, which moves a small axial distance relative to housing 141 before being stopped against further axial displacement. Needle 160 has a sharp needle tip 164 configured to penetrate through first septum 128 and second septum 148. Once needle 160 is stopped from further axial movement, carrier 150 moves downwardly over needle 160 until needle tip 164 penetrates through second septum 148, at which time the needle tip immediately enters first septum 128. During this movement, side opening 162 of needle 160 passes through second septum 148 and immediately into first septum 128. Therefore, side opening 162 remains sealed off from interior and exterior areas of CSTD 100 after emerging from second septum 148 and passing into first septum 128.

Side opening 162 moves through three sealed positions relative to the other components as carrier 150 moves downwardly over needle 160. In the first sealed position, shown in FIG. 3, side opening 162 is sealed between second septum 148 and third septum 158. In the second sealed position, side opening 162 is sealed inside second septum 148. In the third sealed position, side opening 162 is sealed inside first septum 128. Once carrier 150 bottoms out in the receptacle, first septum 128 is pushed down past side opening 162, so that side opening 162 emerges from the first septum and becomes exposed in first passage 126 of first adaptor 120. In this state, side opening 162 forms a fluid path of communication between first adaptor 120, second adaptor 140, and their respective reservoirs to which the adaptors are connected.

First projection 128g on first septum 128 abuts with second projection 148g on second septum 148 to form a dry break coupling when carrier 150 reaches the second position. The elastomeric material of first septum 128 and second septum 148 compress together, which automatically closes and seals off spaces between needle opening 162 and interior spaces CSTD 100 so that liquid and vapor cannot spill, leak or escape from the device.

Second adaptor 140 has a pair of fixed locking ramps 170 along inner wall 144. Carrier 150 has a corresponding pair of lugs 151. Lugs 151 are configured to slidingly engage locking ramps 170 as carrier 150 moves to the second position. Once carrier 150 reaches the second position, lugs 151 lockingly engage ramps 170. This engagement locks CSTD 100 in a “fluid path open” state in which needle 160 provides a fluid path between the first adaptor 120 and second adaptor 140 and their respective reservoirs.

Each lug 151 projects radially outwardly on a flexible arm 156. As carrier 150 is pushed toward the second position, lugs 151 contact locking ramps 170. Locking ramps 170 have ramp surfaces 171 that extend radially inwardly in hollow interior 143 of housing 141 as the ramp surfaces extend toward female Luer connector 180. This orientation of ramp surfaces 171 causes lugs 151 and flexible arms 156 to flex radially inwardly, with energy stored in the flexible arms. Lugs 151 slide along ramp surfaces 171 as carrier 150 is moved toward the second position, bending further radially inwardly until the lugs pass over the ends of locking ramps 170. At such time, carrier 150 bottoms out in housing 141, reaching the second position shown in FIG. 4. In addition, locking ramps 170 no longer contact lugs 151, allowing the lugs to snap outwardly to a relaxed state as energy is released from flexible arms 156. In this state, lugs 151 engage locking ramps 170 to prevent carrier 150 from being moved back toward the first position. This locks carrier 150 in the second position, with the device locked in the “fluid path open” state described earlier.

After fluid is transferred through CSTD 100, first adaptor 120 remains locked inside the second adaptor 140 by the engagement of locking ramps 170 and lugs 151. First adaptor 120 can be released from second adaptor 140 by applying radially inward forces F to a pair of side buttons 142 that are built into the side walls of housing 141. The directions of the forces F are shown by arrows in FIG. 4.

As side buttons 142 are pressed radially inwardly, the side buttons push lugs 151 radially inwardly until the lugs are no longer axially aligned with locking ramps 170. At this stage, locking ramps 170 no longer prevent carrier 150 from being moved from the second position back toward the first position. Therefore, first adaptor 120 can be removed from hollow interior 143 of second adaptor 140 by pressing side buttons 142 inwardly to release carrier 150, and pulling the first adaptor out of the hollow interior and socket 145.

As first adaptor 120 is withdrawn from hollow interior 143, ledge portions 125 on the first adaptor remain engaged with the undersides of clip ends 153. This engagement causes the carrier 150 to be towed or pulled back to the first position in housing 141 as first adaptor 120 is withdrawn from the housing. When carrier 150 reaches the first position, clip ends 153 exit the narrower section 143b of hollow interior 143 and enter wider section 143a. This causes clip ends 153 to snap outwardly to the position shown in FIG. 3. The outward movement of clip ends 153 releases the clip ends from ledge portions 125 on first adaptor 120. Therefore, ledge portions 125 are freed from the clips 152, allowing first adaptor 120 to be pulled completely out of housing 141 and separated from second adaptor 140. Carrier 150 is prevented from being pulled out of housing 141 by lugs 151 which engage undercuts 141b in inner wall 141a of housing 141.

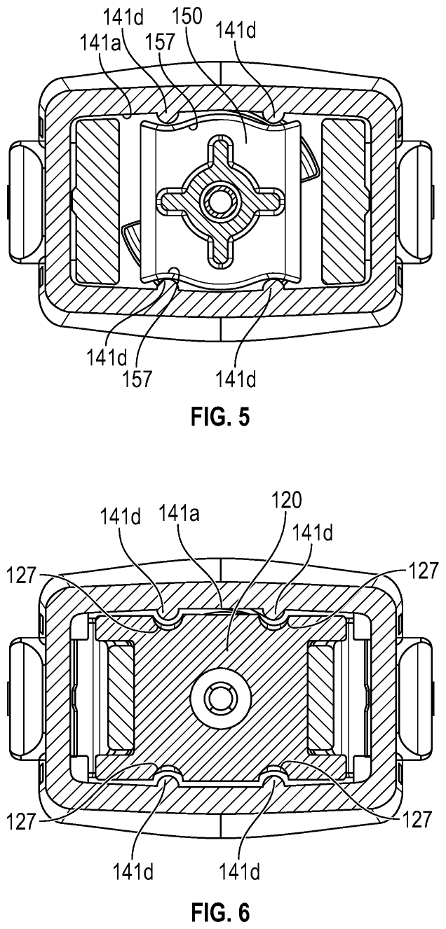

CSTDs according to the present disclosure can include one or more alignment structures that maintain components in proper axial and radial alignment as the components move relative to one another. For example, CSTD 100 includes longitudinal ribs 141d along inner wall 141a of housing 141, which are partially shown in FIG. 2. Ribs 141d are configured to engage longitudinally extending indents 157 on carrier 150, as shown in FIG. 5, to maintain correct alignment between the carrier and housing 141. In addition, ribs 141d are configured to engage longitudinally extending slots 127 on first adaptor 120, as shown in FIG. 6, to maintain correct alignment between the first adaptor and housing 141.

CSTD 100 has features and properties that are structurally and/or functionally identical to features and properties on other embodiments described in the present disclosure. Therefore, some features described on CSTD 100 will not be described on the other embodiments for purposes of brevity, with the understanding that such features are also present on the other embodiments.

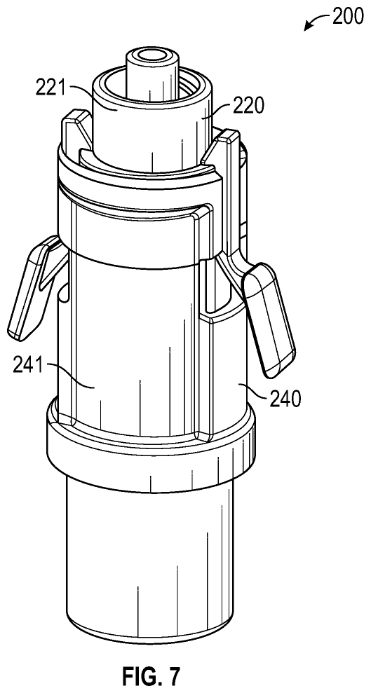

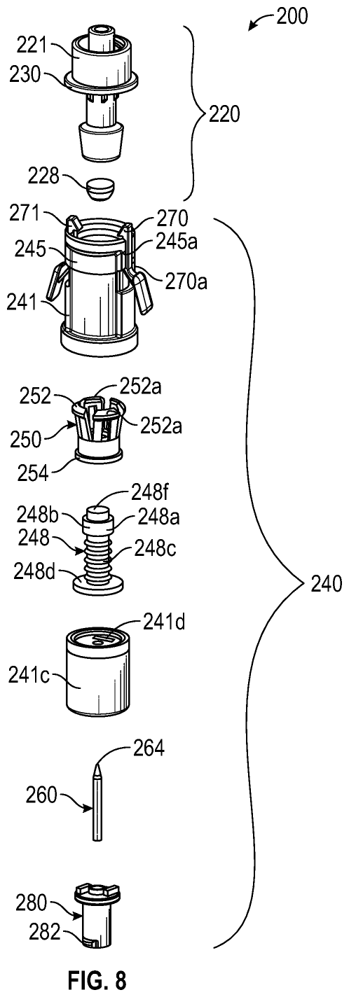

Referring to FIGS. 7 and 8, a CSTD 200 is shown according to a second embodiment. CSTD 200 has a first adaptor 220 configured to attach to a first fluid reservoir and a second adaptor 240 configured to attach to a second fluid reservoir. First adaptor 220 can be connected to a vial, bag, or patient line, for example. Second adaptor 240 has a female Luer connector 280 that can be connected to a syringe or any male Luer connector on a fluid delivery device, such as a pump. Once first adaptor 220 and second adaptor 240 are attached to first and second fluid reservoirs, respectively, and interconnected to each other in the coupled state shown in FIG. 7, CSTD 200 forms a closed fluid passage between the first and second fluid reservoirs.

First adaptor 220 has a generally cylindrical body 221. Second adaptor 240 has a generally cylindrical receptacle or housing 241. Housing 241 of second adaptor 240 is adapted to receive body 221 of first adaptor 220 to interconnect the two adaptors. The cylindrical geometries of the first adaptor 220 and second adaptor 240 allow the first adaptor to be inserted into the second adaptor in any orientation relative to the second adaptor. No specific orientation is required to properly insert first adaptor 220 into second adaptor 240 for connecting the two. This makes connection of the adaptors simple and results in less chance for user error in connecting the adaptors.

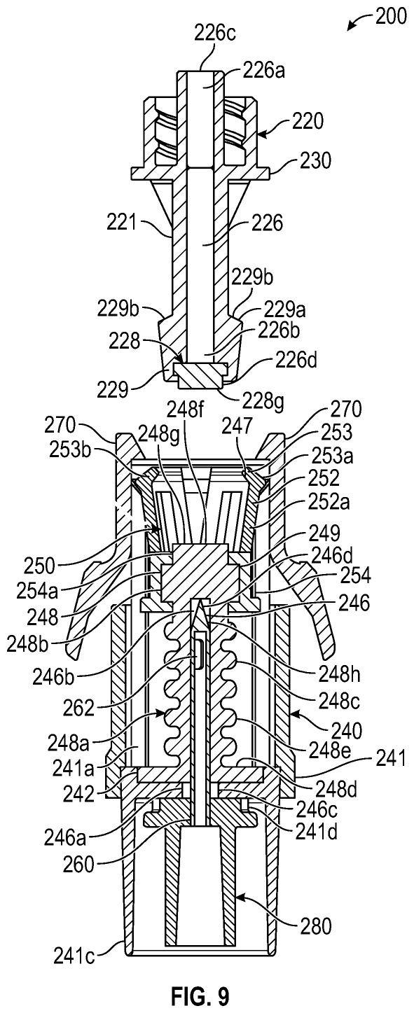

FIG. 9 shows a cross section of first adaptor 220 and second adaptor 240 prior to being coupled together. First adaptor 220 has a first passage 226 and a first septum 228 made of elastomeric material. First passage 226 has a first passage end 226a and a second passage end 226b opposite the first passage end. First passage end 226a defines a first opening 226c and second passage end 226b defines a second opening 226d. First passage 226 widens at second passage end 226b to form a cylindrical chamber or seat 229. First septum 228 is received in seat 229 to seal the second passage end 226b. A portion of first septum 228 extends axially from second passage opening 226d, forming a first projection 228g.

Second adaptor 240 has a second passage 246 and a second septum 248 made of elastomeric material. Second passage 246 has a first passage end 246a and a second passage end 246b opposite the first passage end. First passage end 246a defines a first opening 246c and second passage end 246b defines a second opening 246d. Second passage 246 widens at the second passage end 246b to form a cylindrical chamber or seat 249. A portion of second septum 248 is received in seat 249 to seal the second passage end 246b.

Second septum 248 has an elongated cylindrical body that operates as a collapsible piston 248a. Piston 248a has a head portion 248b, a collapsible midsection 248c, and a base flange 248d. Collapsible midsection 248c has a smaller diameter than head portion 248b and base flange 248d. A series of circumferential ribs 248e extend along the length of collapsible midsection 248c. Base flange 248d is fitted into a cylindrical recess 242 in housing 241.

Head portion 248b of second septum 248 is contained in a carrier 250. Carrier 250 has a collet portion 252 and a hollow cylindrical base portion 254. Base portion 254 defines the cylindrical chamber or seat 249 mentioned earlier. Cylindrical chamber or seat 249 is sized so that head portion 248b engages the interior of base portion 254 in a fluid tight fit. Base portion 254 has an opening 254a that interconnects cylindrical chamber or seat 249 with an interior of collet portion 252. An end portion 248f of second septum 248 protrudes through opening 254a, forming a second projection 248g that extends into collet portion 252. Second projection 248g is configured to abut with and be deformed by first projection 228g, and vice versa, to form a dry break coupling.

Carrier 250 is configured to slide axially inside housing 241 between a first position and a second position. FIG. 9 shows carrier 250 in the first position. Collet portion 252 has four flexible clips 252a that are made of a resilient flexible material. In the relaxed state, clips 252a extend radially outwardly in a splayed arrangement. Each clip 252a has a clip end 253 with an outer projection 253a that extends radially outwardly and an inward detent 253b that extends radially inwardly. Each outer projection 253a rests against a tapered landing 247 in housing 241 when the carrier 250 is in the first position. Outer projections 253a engage landing 247 to maintain carrier 250 in the first position.

First adaptor 220 is connected to second adaptor 240 by inserting body 221 of first adaptor 220 into housing 241 of second adaptor 240 and in between clips 252a of carrier 250. Body 221 is advanced until first septum 228 contacts projection 248g of second septum 248. Once first septum 228 contacts second septum 248, movement of carrier 250 out of the first position is resisted by the engagement between clips 252a and landing 247. This resistance is overcome by applying manual force to first adaptor 220 in the axial direction toward second septum 248 until the manual force exceeds the resistance force.

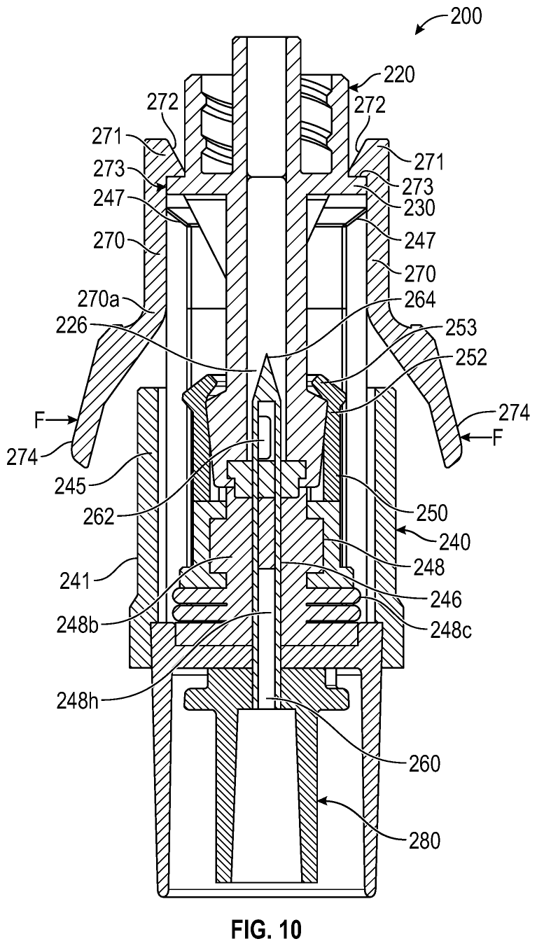

Seat 229 in first adaptor 220 is surrounded by a wedge-shaped plug section 229a. Plug section 229a passes between clips 252a as first adaptor 220 moves into collet portion 252. Inner wall 241a of housing 241 tapers radially inwardly as the inner wall extends away from landings 247. Clip ends 253, which bear against inner wall 241a, are compressed radially inwardly under stored energy by the inner wall as carrier 250 is moved out of the first position toward the second position. This radial compression pushes detents 253b radially inwardly against a shoulder 229b that extends circumferentially around plug section 229a. The engagement between detents 253b and shoulder 229b temporarily interlocks carrier 250 and first adaptor 220. In this condition, carrier 250 is free to move from the first position in housing 241 toward the second position. As carrier 250 moves toward the second position, collapsible midsection 248c is compressed axially. FIG. 10 shows first adaptor 220 inserted fully into second adaptor 240, with carrier 250 moved to the second position. In this position, clips 252s are flexed radially inwardly under stored energy, and collapsible midsection 248c is compressed axially under stored energy.

Second adaptor 240 houses a needle 260, as shown in FIGS. 9 and 10. Needle 260 is axially fixed to female Luer connector 280. Female Luer connector 280 is axially movable relative to housing 241 through a small axial distance, but is restricted from moving beyond a small range of motion by a snap fit arrangement or other captive configuration, as described in the first embodiment. In this arrangement, needle 260 is captively held in housing 241 of second adaptor 240, and only permitted to move a small axial distance relative to the housing before being stopped from further axial movement. In contrast, carrier 250 and head portion 248b of second septum 248 are axially movable relative to housing 241 through most of the length of the housing.

First septum 228 and second septum 248 are formed of elastomeric material that can be pierced by needle 260 as carrier 250 is pushed toward the second position. Needle 260 has a side opening 262 that remains sealed inside second septum 248 prior to coupling second adaptor 240 to first adaptor 220. As first adaptor 220 is advanced into housing 241, first septum 228 pushes against second septum 248 and moves head portion 248b and carrier 250 downwardly in the housing. Head portion 248b and carrier 250 are moved downwardly over needle 260, which moves a small axial distance relative to housing 241 before being stopped against further axial displacement by a snap fit arrangement or other captive configuration, as described in the first embodiment. Needle 260 has a sharp needle tip 264 configured to penetrate through first septum 228 and head portion 248b. Once needle 260 is stopped from further axial movement, carrier 250 moves downwardly over needle 260 until needle tip 264 penetrates through second septum 248 and enters first septum 228. During this movement, side opening 262 of needle 260 passes through second septum 248 and immediately into first septum 228.

Side opening 262 moves through three sealed positions relative to the other components as carrier 250 moves downwardly over needle 260. In the first sealed position, side opening 262 is sealed inside a narrow hollow core 248h inside collapsible midsection 248c of second septum 248. This position is shown in FIG. 9. In the second sealed position, side opening 262 is sealed inside head portion 248b of second septum 248. In the third sealed position, side opening 262 is sealed inside first septum 228. Once carrier 250 bottoms out in second adaptor 240, first septum 228 is pushed down past side opening 262, so that the side opening emerges from the first septum and becomes exposed in first passage 226 of first adaptor 220, shown in FIG. 10. In this state, side opening 262 forms a fluid path of communication between second adaptor 240 and first adaptor 220. First septum 228 and second septum 248 form a dry break coupling when carrier 250 reaches the second position.

First adaptor 220 has a circumferential flange 230 that extends radially outwardly from body 221. Second adaptor 240 has a pair of retaining clips 270 that are pivotally connected to the wall 245 of housing 241. Each retaining clip 270 is pivotally connected to wall 245 on an elastic hinge 270a. Hinges 270a allow each retaining clip 270 to pivot through openings 245a in wall 245. Flange 230 on first adaptor 220 passes between retaining clips 270 as the first adaptor is inserted into second adaptor 240. Retaining clips 270 are formed of resilient flexible material and define a space between them in a relaxed state, which is shown in FIG. 9. Each retaining clip 270 has a barb-shaped clip end 271 with two engagement surfaces. The first engagement surface is a ramped contact surface 272 facing radially inwardly on the second adaptor. The second engagement surface is an undercut surface 273 that extends perpendicular to the longitudinal axis.

The diameter of flange 230 is larger than the space between clip ends 271 when retaining clips 270 are in the relaxed state. Therefore, flange 230 bears against the ramped contact surfaces 272 as it passes between the retaining clips 270. The orientations of the ramped contact surfaces 272 are such that axial force applied to first adaptor 220 toward second adaptor 240 displaces retaining clips 270 radially outwardly with respect to the axis of the second adaptor. Retaining clips 270 flex radially outwardly and spread apart under stored energy to permit the flange to pass through ramped contact surfaces 272.

Once flange 230 clears ramped contact surfaces 272, clips 270 are no longer subject to the force that displaces them outwardly. Therefore, clips 270 snap back to the relaxed state as energy is released, with the undercut surfaces 273 of the clips positioned over flange 230, as shown in FIG. 10. The axial engagement between undercut surfaces 273 and flange 230 prevents first adaptor 220 from being removed from second adaptor 220, thereby locking them together. The moment that clips 270 snap over flange 230 preferably coincides with side opening 262 emerging completely from the first septum where it is exposed in first passage 226. It also preferably coincides with carrier 250 reaching the second position. Therefore, when carrier 250 reaches the second position, first adaptor 220 and second adaptor 240 are locked together in a “fluid path open” state in which the needle provides a fluid path between the first and second adaptors and their respective reservoirs. Clips 270 bear against flange 230 to lock first adaptor 220 and second adaptor 240 together while resisting an axial expansion force being exerted by collapsible midsection 248c in its compressed state. This results in the first adaptor 220 and second adaptor 240 being locked together under tension. The snapping of clips 270 over flange 230 creates an audible click that notifies the user when CSTD 200 is locked in the fluid path open state.

After fluid is transferred through CSTD 200, first adaptor 220 remains locked inside second adaptor 240 by the engagement between retaining clips 270 and flange 230. First adaptor 220 can be released from second adaptor 240 by applying radially inward forces F to a pair of side buttons 274 that extend radially outwardly from retaining clips 270. The directions of the forces F are shown by arrows in FIG. 10.

As side buttons 274 are pressed radially inwardly, retaining clips 270 pivot through openings 245a in wall 245 and store energy at the hinges 270a. Clip ends 271 are pivoted radially outwardly and away from first adaptor 220, so that the clips ends are moved out of their locking positions to release positions. In the release positions, the undercut surfaces 273 no longer obstruct flange 230 on first adaptor 220, thereby permitting the flange to be withdrawn from retaining clips 270, and allowing the first adaptor to be withdrawn from second adaptor 240.

Clip ends 253 of carrier 250 are pressed inwardly and wrap around plug section 229a of first adaptor 220, as noted above. Therefore, withdrawal of first adaptor 220 also tows or withdraws carrier 250 back toward the first position. Once clip ends 253 align axially with landing 247, the stored energy in the clip ends is released, causing the clip ends to expand and return to their relaxed state shown in FIG. 9. This releases plug section 229a from the grip of collet portion 252, allowing separation of first adaptor 220 from second adaptor 240.

When side buttons 274 are pressed inwardly to release flange 230 from clips 270, the force holding the collapsed piston 248a in the compressed state is removed. Therefore, stored energy in the collapsible midsection 248c of second septum 248 is released, causing the collapsible midsection to expand. As collapsible midsection 248c expands, head portion 248b applies a spring force to carrier 250 that aids in the return of the carrier to the first position. The spring force also propels head portion 248b back to the original position to immediately enclose needle tip 264 inside second septum 248 after first septum 228 is moved off of the needle tip. This provides a safety feature that shields needle tip 264 after first adaptor 220 is removed from housing 241. Needle tip 264 is never exposed before, during or after use of CSTD 200.

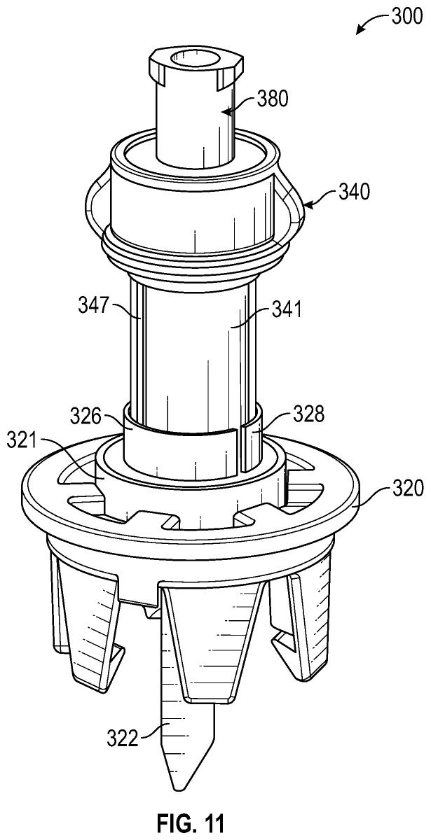

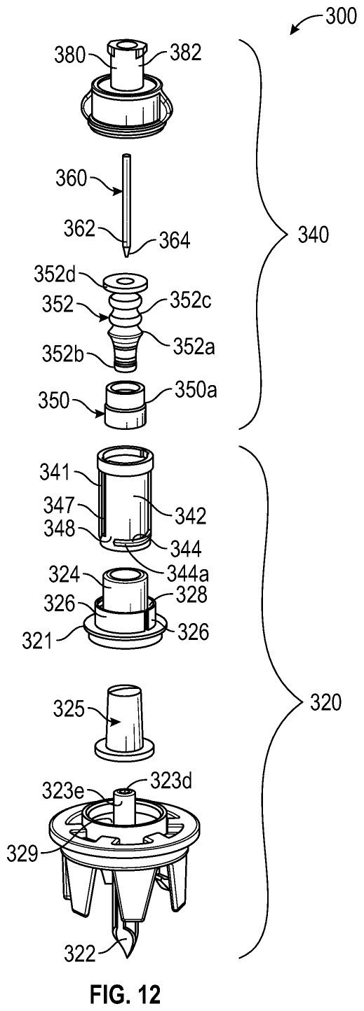

Referring to FIGS. 11 and 12, a CSTD 300 is shown according to a third embodiment. CSTD 300 has a first adaptor 320 configured to attach to a first fluid reservoir and a second adaptor 340 configured to attach to a second fluid reservoir. First adaptor 320 has a spike 322 that can be connected to a vial, for example. Second adaptor 340 has a female Luer connector 380 that can be connected to a syringe or any male Luer connector on a fluid delivery device, such as a pump. A needle 360 is housed inside second adaptor 340 and forms a fluid conduit that communicates with first adaptor 320 as will be explained. Once first adaptor 320 and second adaptor 340 are attached to first and second fluid reservoirs, respectively, and interconnected to each other in the coupled state shown in FIG. 11, CSTD 300 forms a closed fluid passage between the first and second fluid reservoirs.

First adaptor 320 has a generally cylindrical body 321. Second adaptor 340 has a generally cylindrical receptacle or housing 341 with a cylindrical wall 342. Housing 341 of second adaptor 340 is adapted to receive body 321 of first adaptor 320 to interconnect the two adaptors. First and second adaptors 320, 340 are connected by inserting the first adaptor into the second adaptor with an axial push, followed by rotation or twisting of one adaptor relative to the other to lock the first and second adaptors together.

First adaptor 320 has a cylindrical plug 324 and a plurality of resilient flexible snap arms 326 arranged circumferentially around the plug. Snap arms 326 are spaced from the exterior of plug 324 by small radial gaps 328. Cylindrical wall 342 of second adaptor 340 has an inner diameter that is larger than the outer diameter of plug 324. The inner diameter between snap arms 326 is larger than the outer diameter of cylindrical wall 342. In this arrangement, housing 341 is sized to telescopically receive plug 324. In addition, radial gaps 328 between plug 324 and snap arms 326 are sized to telescopically receive cylindrical wall 342.

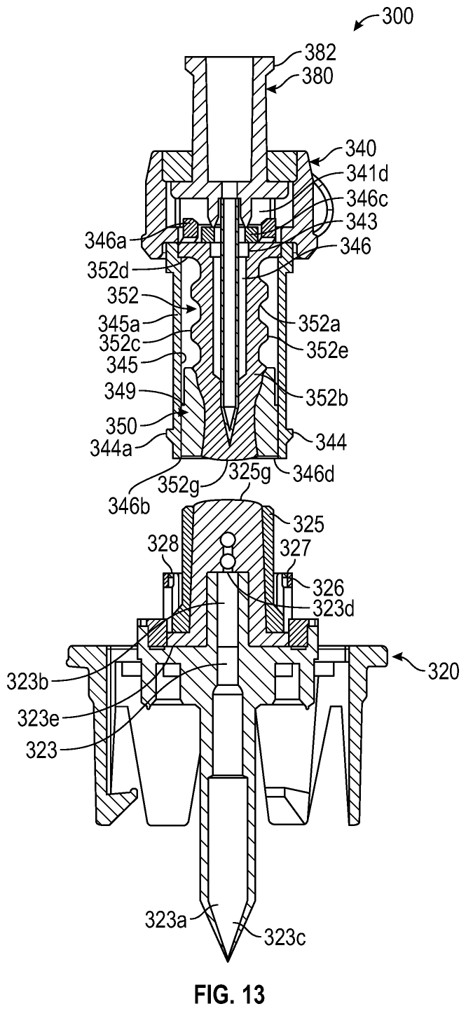

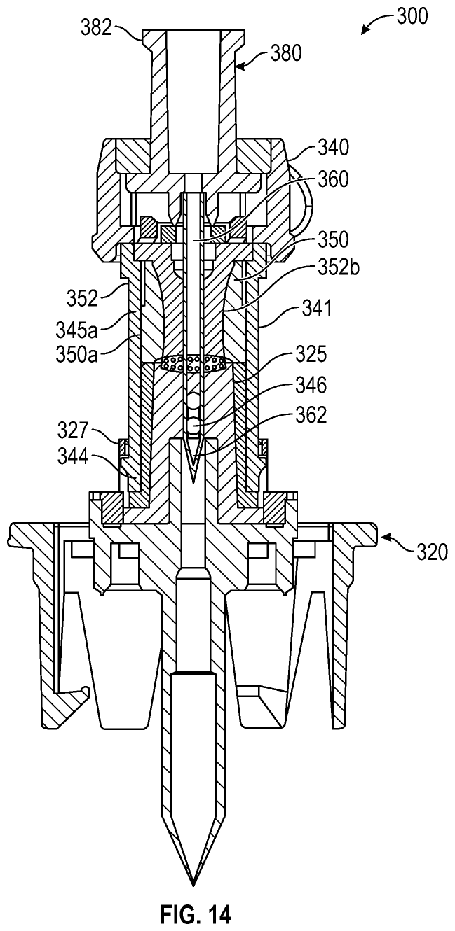

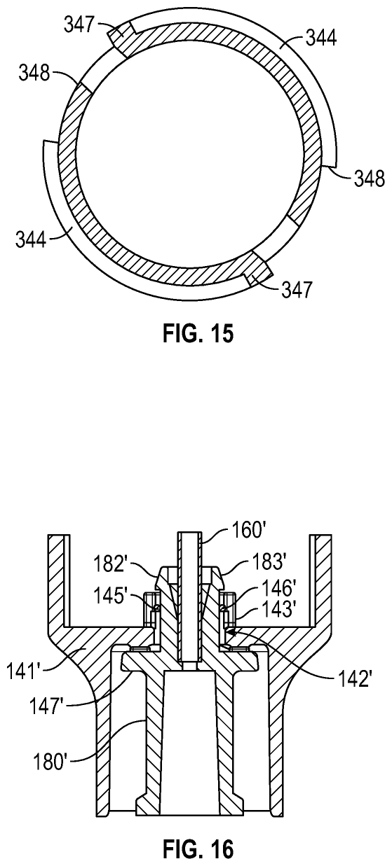

Snap arms 326 of first adaptor 320 are configured to snap over a ledge 344 on second adaptor 340 once the first adaptor is inserted fully into second adaptor. Referring to FIGS. 13-15, ledge 344 projects radially outwardly from cylindrical wall 342 along a portion of its circumference, and has a tapered outer sidewall 344a. Each snap arm 326 has barbed end 327 with a tapered leading face 328. Tapered leading face 328 of each snap arm 326 is configured to contact the tapered outer sidewall 344a of ledge 344 as first adaptor 320 is inserted into second adaptor 340.

The orientation of each tapered leading face 328 is angled so that contact between the tapered leading face and ledge 344 causes the snap arm 326 to splay or flex radially outwardly. As first adaptor 320 advances into second adaptor 340, snap arms 326 flex radially outwardly under stored energy until barbed ends 327 pass over and clear ledge 344. Once barbed ends 327 clear ledge 344, the stored energy in snap arms 326 is released, causing the snap arms to snap radially inwardly with the barbed ends hooked over the ledge.

Once first adaptor 320 is fully inserted into second adaptor 340, the first adaptor is rotatable relative to the second adaptor between axially locked positions, which consist of a range of orientations relative to the second adaptor, and an axially unlocked position. In the axially locked positions, barbed ends 327 of snap arms 326 are hooked over ledge 344. In the axially unlocked position, first adaptor 320 is oriented relative to second adaptor 340 so that barbed ends 327 of snap arms 326 are only aligned with openings or passages 348 through ledge 344. Passages 348 break ledge 344 into arcuate-shaped ledge sections 344a and are arranged around the ledge so as to align with snap arms 326 when first adaptor 320 is rotated to a specific orientation relative to second adaptor 340. First adaptor 320 and second adaptor 340 can have any number of passages, ledge sections and snap arms, and the example shown in the Figs. does not represent the only arrangement contemplated.

Each passage 348 is wide enough to allow at least one barbed end 327 of a snap arm 326 to pass through the passage once first adaptor 320 is rotated to the unlocked position. The locked and unlocked positions of first adaptor 320 can be detected visually and by tactile feel. Second adaptor 340 has a pair of diametrically opposed hard stops 347 that project from cylindrical wall 342 and extend axially. Hard stops 347 are positioned relative to the ledge sections 344a and passages 348 to create rotation limiters after first adaptor 320 is inserted into second adaptor 340. After first adaptor 320 is fully inserted into second adaptor 340, the first adaptor is rotated clockwise until one of the barbed ends 327 collides with one of the hard stops 347. In this relative orientation, at least some of the barbed ends 327 are hooked over ledge 344, preventing first adaptor 320 from being pulled out of second adaptor 340. The hard stop 347 that collides with the barbed end 327 after clockwise rotation provides a tactile indicator that the first adaptor is locked to the second adaptor.

First adaptor 320 can also be rotated in a counterclockwise direction in a similar manner until one of the barbed ends 327 collides with the other hard stop 347. In this relative orientation, all of the barbed ends 327 are axially aligned with a passage 348, allowing first adaptor 320 to be pulled out of second adaptor 340. Therefore, the hard stop 347 that is encountered after a counterclockwise rotation provides a tactile indicator that first adaptor 320 is unlocked from second adaptor 340 and can be pulled out of the second adaptor.

The cylindrical geometries of first adaptor 320 and second adaptor 340 allow the first adaptor to be inserted into the second adaptor in any orientation relative to the second adaptor. No specific orientation is required to properly insert first adaptor 320 into second adaptor 340 for connecting the two. This makes connection of the adaptors simple and results in less chance for user error in connecting the adaptors. However, first adaptor 320 must be rotated clockwise until one of the hard stops 347 is encountered to ensure that the first adaptor is locked, and rotated counterclockwise until another of the hard stops 347 is encountered to determine when the first adaptor is unlocked.

FIG. 13 shows a cross section of first adaptor 320 and second adaptor 340 prior to being coupled together. First adaptor 320 has a first passage 323 and a first septum 325 made of elastomeric material. First passage 323 has a first passage end 323a and a second passage end 323b opposite the first passage end. First passage end 323a defines a first opening 323c and second passage end 323b defines a second opening 323d. An annular seat 329, shown best in FIG. 12, is formed around an exterior wall 323e surrounding second passage end 323b. First septum 325 is received in seat 329 and closes second opening 323d to seal second passage end 323b.

Second adaptor 340 has a second passage 346 and a second septum 352 made of elastomeric material. Second passage 346 has a first passage end 346a and a second passage end 346b opposite the first passage end. First passage end 346a defines a first opening 346c and second passage end 346b defines a second opening 346d. Second passage end 346b forms a cylindrical chamber or seat 349. Second septum 352 is received in seat 349 to seal the second passage end 346b. First septum 325 has a first projection 325g and second septum 352 has a second projection 352g configured to form a dry break coupling with the first projection, like the other embodiments.

Second septum 352 has an elongated cylindrical body that operates as a collapsible piston 352a. Piston 352a has a head portion 352b, a collapsible midsection 352c, and a base flange 352d. A thermoplastic casing or carrier 350 surrounds head portion 352a. Carrier 350 prevents the elastomer of head portion 352a from expanding diametrically to ensure an adequate seal by allowing pressure to be constantly applied inwardly to needle 360 by the constraining effects of the carrier. Collapsible midsection 352c has a series of circumferential ribs 352e that extend along the length of collapsible midsection 352c. Base flange 352d is fitted into a cylindrical recess 343 in housing 341.

As first adaptor 320 is inserted into the housing 341 of second adaptor 340, first septum 325 compresses against second septum 352. In addition, plug 324 bears axially against carrier 350. Axial forces applied to head portion 352b of second septum 352 and carrier 350 move the second septum and carrier in an axial direction toward base flange 352d of the second septum. In addition, collapsible midsection 352c of second septum 352 collapses in response to the axial load applied to head portion 352b. Carrier 350 is initially disposed in a first position at the mouth of housing 341 and travels to a second position deeper into the housing. During this travel, carrier 350 slides along an inner wall 345 of housing 341. Inner wall 345 abruptly transitions from a larger inner diameter to a smaller inner diameter at a constriction 345a. Constriction 345a forms an end wall or stop that abuts a circumferential stop flange 350a on carrier 350 when the carrier reaches the second position. Therefore, carrier 350 bottoms out in housing 341 when the carrier reaches the second position and can travel no further.

Needle 360 is axially fixed to female Luer connector 380. Female Luer connector 380 is axially movable relative to housing 341 through a small axial distance, but is restricted from moving beyond a small range of motion by a snap fit arrangement or other captive configuration, as described in the first embodiment. In this arrangement, needle 360 is captively held in housing 341, and only permitted to move a small axial distance relative to the housing before being stopped from further axial movement. In contrast, carrier 350 and head portion 352b of second septum 352 are axially movable relative to housing 341 through most of the length of the housing.

First septum 325 and second septum 352 are formed of elastomeric material that can be pierced by needle 360 as carrier 350 and head portion 352b are pushed deeper into housing 341. Needle 360 has a side opening 362 that remains sealed by second septum 352 prior to coupling second adaptor 340 to first adaptor 320 as shown. Needle 360 also has a sharp needle tip 364 configured to penetrate through head portion 352b of second septum 352 and first septum 325.

Collapsible portion 352c of second septum 352 collapses under stored energy in response to axial load as first adaptor 320 displaces carrier 350 and head portion 352b of second septum 352 during insertion of the first adaptor into second adaptor 340. This causes carrier 350 and head portion 352b to travel inside housing 341 toward base flange 352d. Needle 360 moves a small axial distance relative to housing 341 before being stopped against further axial displacement by a snap fit arrangement or other captive configuration, as described in the first embodiment. Once needle 360 is stopped from further axial movement, needle tip 364 pierces through second septum 352 until the tip emerges from the second septum and immediately penetrates first septum 325, which is compressed against the second septum. Side opening 362 of needle 360 also moves from the interior of second septum 352 into first septum 325. Continued advancement causes needle tip 364 and side opening 362 to penetrate through first septum 325 until the needle opening is in fluid communication with first passage 323 in first adaptor 320. At this stage, carrier 350 reaches the second position, and needle 360 forms a fluid passage between first adaptor 320 and second adaptor 340. This substantially coincides with the snap arms 326 snapping over the ledge 344. First adaptor 320 can be rotated clockwise relative to second adaptor 340 at this stage until one of the hard stops 347 is encountered. When one of the hard stops 347 is encountered, first adaptor 320 and second adaptor 340 are locked together in a “fluid path open” state in which needle 360 provides a fluid path between the first and second adaptors and their respective reservoirs.

As noted above, first septum 325 and second septum 352 form a dry break coupling when compressed together. The elastomeric material of first septum 325 and second septum 352 automatically closes and seals off spaces between needle 360 and the interior of CSTD 300 so that liquid cannot spill, leak or escape.

After CSTD 300 is used to transfer liquid between reservoirs, first adaptor 320 remains locked to second adaptor 340 by the engagement between snap arms 326 and ledge 344. To remove first adaptor 320 from second adaptor 340, the user can rotate the first adaptor relative to the second adaptor in a counterclockwise direction until the other of the hard stops 347 is encountered. At this stage, snap arms 326 are aligned with passages 348 through ledge 344, allowing the snap arms to pass through the passages and facilitate separation of the first adaptor from the second adaptor.

When first adaptor 320 and second adaptor 340 are locked together, stored energy in collapsible midsection 352c creates axial spring force that holds the first and second adaptors together under tension. When first adaptor 320 is rotated to the unlocked position and withdrawn from second adaptor 340, stored energy in the collapsible midsection 352c is released, creating spring force that expands the collapsible midsection and propels head portion 352b of second septum 352 back over needle tip 364 of needle 360. This provides a safety feature that shields needle tip 364 after first adaptor 320 is removed from second adaptor 340. Needle tip 360 is not exposed to the atmosphere where it can directly contact a user at any time before, during or after proper use of CSTD 300.

CSTDs according to the present disclosure can have a variety of couplings and attachment structures for connecting each adaptor to a fluid reservoir. For example, the female Luer connectors 180, 280 and 380 are rotatably mounted to their surrounding housings and feature a thread 182, 282, 382, respectively. Housings 141 and 241 feature a shroud 141c and 241c that extend around Luer connector 180 and 280, respectively. The housings also have ratchet mechanisms 141d, 241d, 341d that comprise ramps that cooperatively engage tabs on female Luer connectors 180, 280 and 380, respectively. The threads 182, 282, 382 and ratchet mechanisms 141d, 241d, 341d allow male Luer connectors to be screwed onto the female Luer connectors in a first direction (e.g. clockwise), but prevent the male Luer connectors from being unscrewed from the female Luer connectors in a second direction opposite the first direction (e.g. counterclockwise). This prevents the user from disconnecting the syringe (or other reservoir) from the second adaptor after liquid has been transferred through the device. The thread and ratchet mechanism can have a variety of configurations, including but not limited to the configurations described in Applicant's U.S. Pat. Nos. 7,857,805 and 5,328,474, the contents of both patents being incorporated by reference herein in their entireties.

Housings and shrouds according to the present disclosure can be manufactured as separately formed parts. For example, CSTD 100 has a housing 141 and a separately formed shroud 141c that are assembled together. In alternative embodiments, the housing and shroud can be manufactured as a one-piece component.

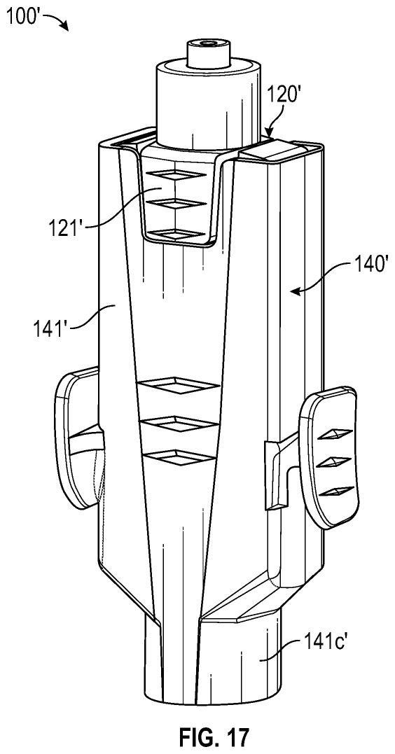

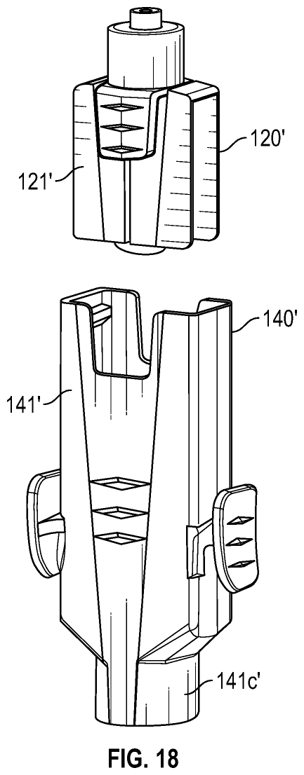

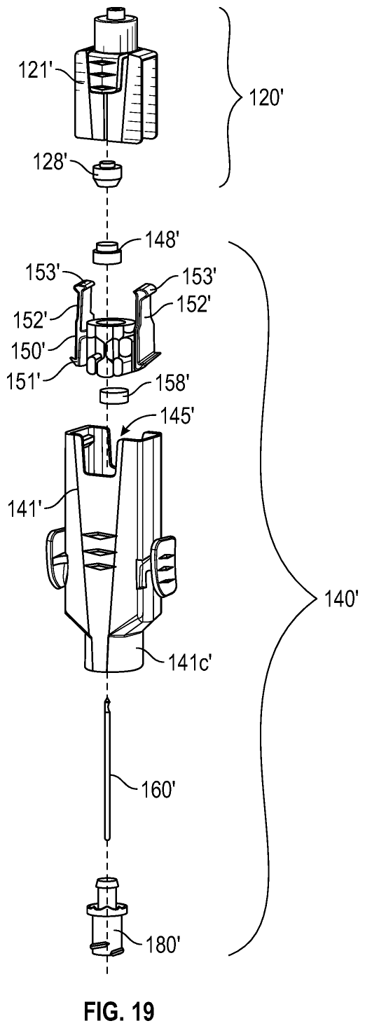

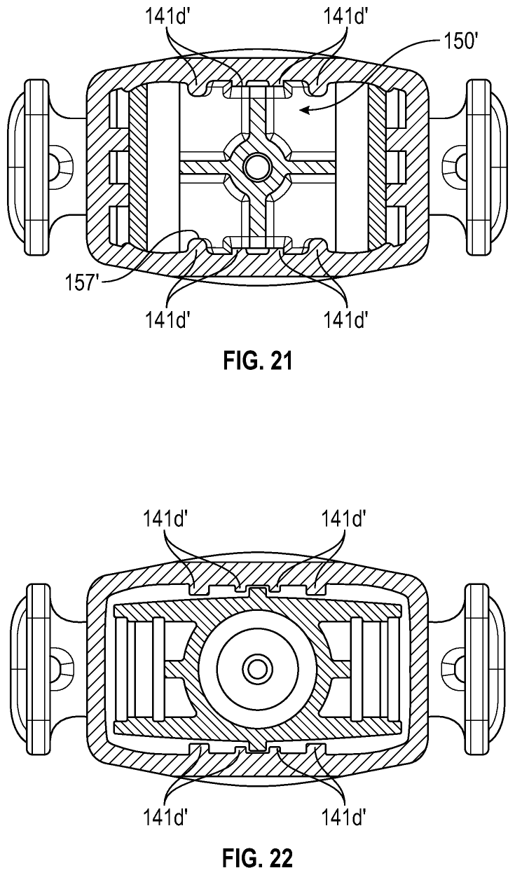

Referring to FIGS. 17-22, a CSTD 100′ is shown according to another embodiment, in which the housing and shroud are manufactured as a one-piece component. Features of CSTD 100′ that correspond to features of CSTD 100 are labeled with the same reference signs followed by a prime symbol (′). Some features of CSTD 100′ that are present in identical or equivalent form in a previously described embodiment(s) will not be described for brevity.

CSTD 100′ has a one-piece housing 141′ that can be injection molded. A shroud 141c′ is integrated with housing 141′ as a single unitary body. This integration reduces the total number of parts and the total number of steps required to assemble CSTD 100′. Two cut-outs extend on opposite sides of socket 145′ as shown. The cut-outs provide greater access to the interior of housing 141′, as compared to conventional adaptors. The greater access to the interior enables easier disinfection of both sides of the device.

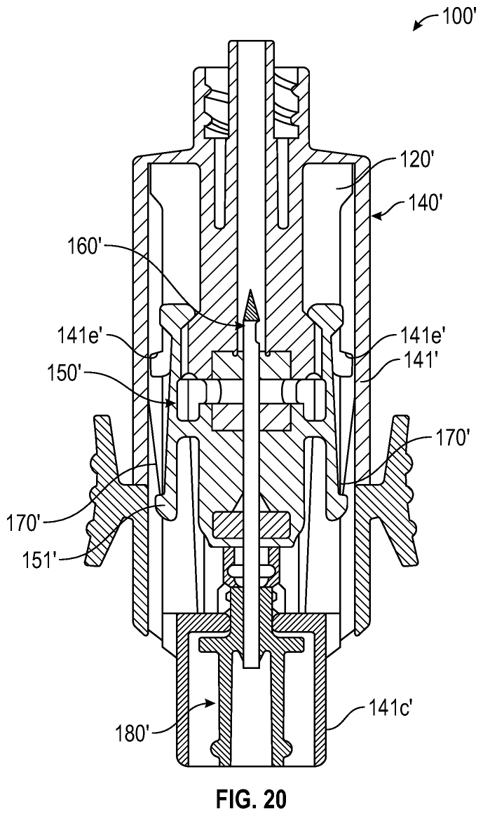

Referring to FIG. 20, CSTD 100′ features a pair undercuts 141e′ in inner wall 141a′ of housing 141′. Undercuts 141e′ engage with lugs 151′ on carrier 150′ to retain the carrier in the first position so that the carrier cannot be removed from housing 141′. Housing 141′ also has a pair of locking ramps 170′ that lockingly engage the lugs 151′ on carrier 150′ to lock the carrier in the second position, as shown in FIG. 20.

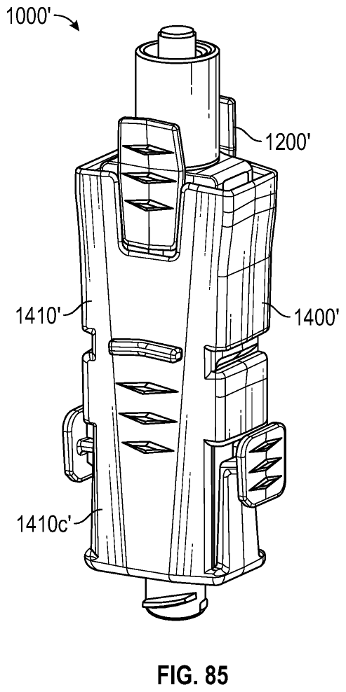

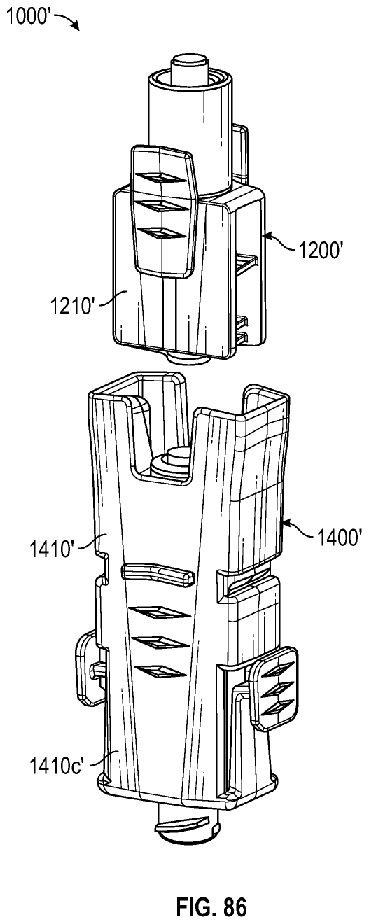

Referring to FIGS. 85-89, a CSTD 1000′ is shown according to another embodiment, in which the housing and shroud are manufactured as a one-piece component. Features of CSTD 1000′ that correspond to features of CSTD 100′ are labeled with the same reference numbers multiplied by 10 and followed by a prime symbol (′). Some features of CSTD 1000′ that are present in identical or equivalent form in a previously described embodiment(s) will not be described for brevity.

CSTD 1000′ has a one-piece housing 1410′ that can be injection molded. A shroud 1410c′ is integrated with housing 1410′ as a single unitary body. The integration of shroud 1410c′ and housing 1410′ reduces the total number of parts and the total number of steps required to assemble CSTD 1000′.

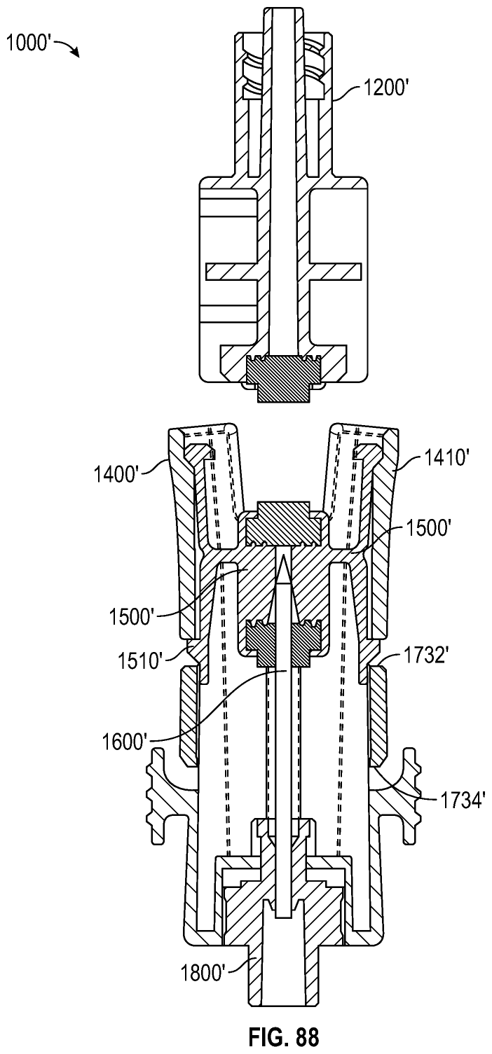

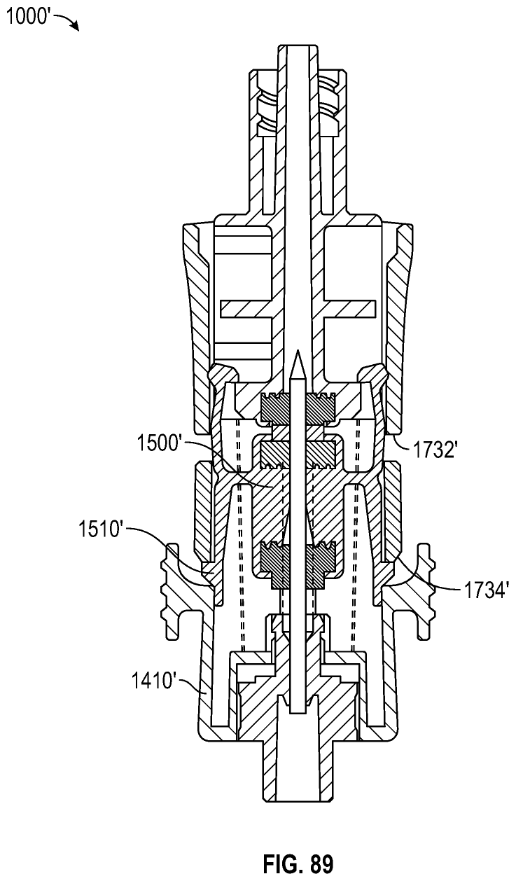

CSTD 1000′ differs from CSTD 100′ in the mechanisms used to retain the carrier 1500′ in the first position and second position. Referring to FIGS. 88 and 89, housing 1410′ features a pair of first locking windows 1732′ that extend through housing 1410′, and a pair of second locking windows 1734′ that extend through the housing. First locking windows 1732′ lockingly engage a pair of lugs 1510′ on carrier 1500′ to retain the carrier in the first position. Second locking windows 1734′ lockingly engage lugs 1510′ on carrier 1500′ to retain the carrier in the second position, as shown in FIG. 89. Lugs 1510′ are visible through first locking windows 1732′ and second locking windows 1734′ from the outside of CSTD 1000′, thereby providing a visible indicator of the position of carrier 1500′ and the operating state of the device.

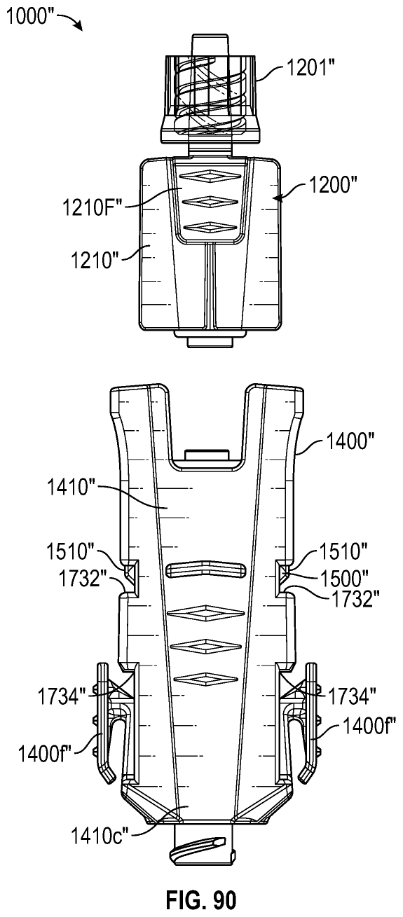

Referring to FIGS. 90-92, a CSTD 1000″ is shown according to another embodiment, in which the housing and shroud are manufactured as a one-piece component. Features of CSTD 1000″ that correspond to features of CSTD 1000′ are labeled with the same reference numbers followed by two prime symbols (″). Some features of CSTD 1000″ that are present in identical or equivalent form in a previously described embodiment(s) will not be described for brevity.

CSTD 1000″ has a first adaptor 1200″ configured to attach to a first fluid reservoir and a second adaptor 1400″ configured to attach to a second fluid reservoir. First adaptor 1200″ and second adaptor 1400″ are similar to first adaptor 1200′ and second adaptor 1400′ on CSTD 1000′, but have different side flanges 1200f″ and 1400f″ for holding each adaptor. In addition, first adaptor 1200″ has a different connector 1201″ for attachment to a vial or other reservoir.

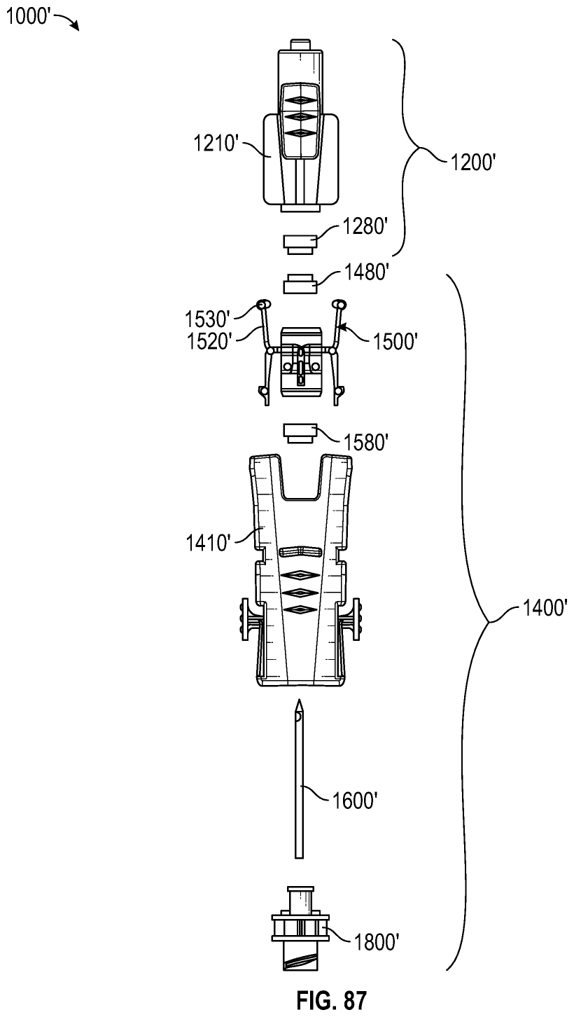

First adaptor 1200″ and second adaptor 1400″ can be interconnected to each other in a coupled and locked state to form a closed fluid passage between the first and second fluid reservoirs. Second adaptor 1400″ has a shroud 1410c″ integrated with a housing 1410″, forming a single unitary body. Housing 1410″ contains a carrier 1500″ with a pair of lugs 1510″ in the same configuration as carrier 1500′ shown in FIGS. 87-89. Carrier 1500″ is slidable in housing 1410″ between a first position and a second position in the same manner as carriers of previous embodiments.

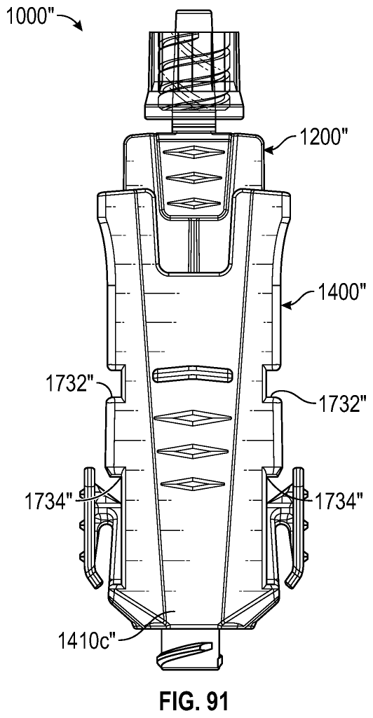

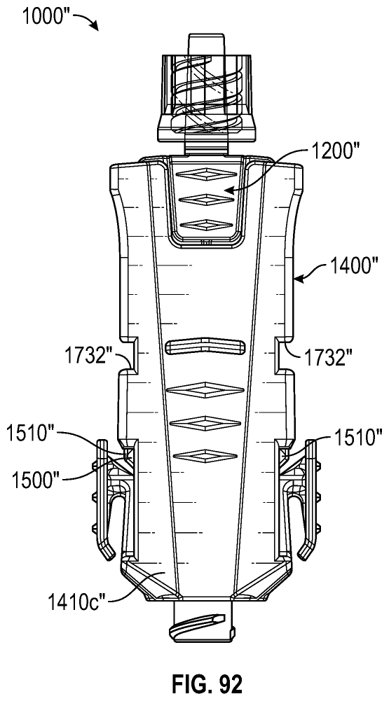

FIG. 90 shows first adaptor 1200″ and second adaptor 1400″ in an uncoupled or “fully disconnected” state, with carrier 1500″ in the first position in housing 1410″. FIG. 91 shows first adaptor 1200″ partially inserted into second adaptor 1400″, with carrier 1500″ moved to an intermediate position (not visible) between the first position and the second position. FIG. 92 shows first adaptor 1200″ completely inserted into second adaptor 1400″, with carrier 1500″ secured in the second position, and the first and second adaptors in a coupled and locked state, or “fully connected” state.

Housing 1410″ has a pair of first locking windows 1732″ that extend through housing 1410″, and a pair of second locking windows 1734″ that open through opposing sides of the housing. First locking windows 1732″ lockingly engage lugs 1510″ on carrier 1500″ to retain the carrier in the first position, as shown in FIG. 90. Second locking windows 1734″ lockingly engage lugs 1510″ on carrier 1500″ to retain the carrier in the second position, as shown in FIG. 92. Lugs 1510″ are visible through first locking windows 1732″ and second locking windows 1734″ from the outside of CSTD 1000″, thereby providing a visible indicator of the position of carrier 1500″ and whether the adaptors are in the fully disconnected state or fully connected state. Lugs 1510″ are only visible through housing 1410″ when carrier is in the first position or in the second position. Therefore, lugs 1510″ can only be seen through housing 1410″ when CSTD 1000″ is in the fully connected state or the fully disconnected state. To enhance the visibility of lugs 1510″, housing 1410″ can be manufactured in one color or shade, and lugs 1510″ can be manufactured in a contrasting color or shade, so that the user can easily see when CSTD 1000″ is in the fully disconnected state or fully connected state.

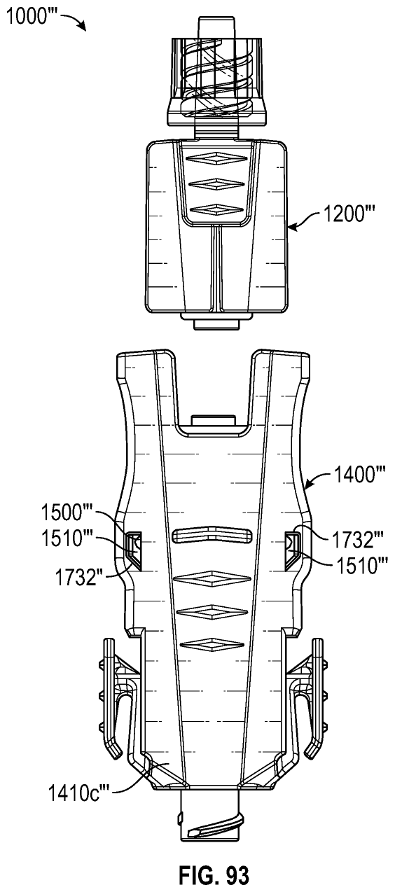

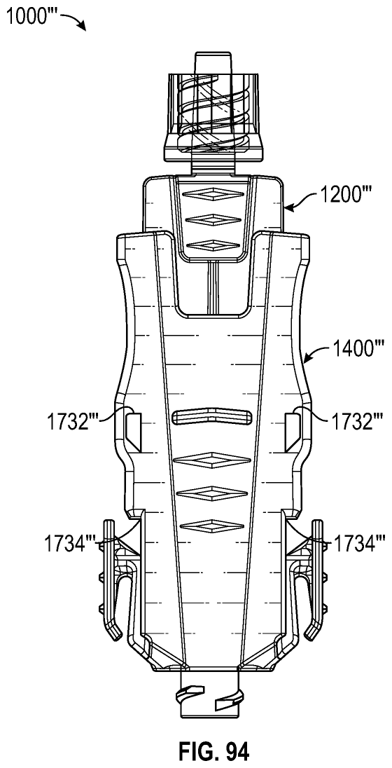

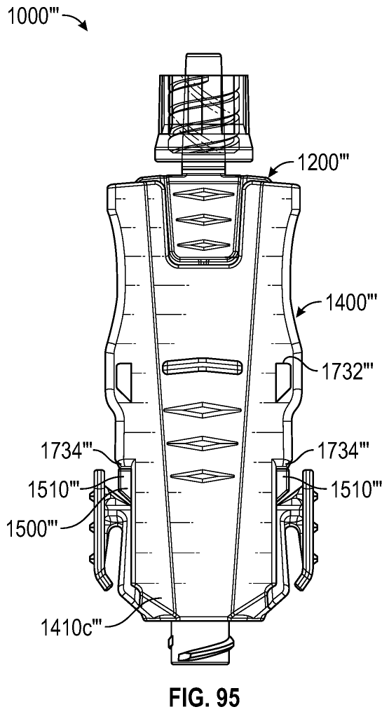

Referring to FIGS. 93-95, a CSTD 1000′″ is shown according to another embodiment, in which the housing and shroud are manufactured as a one-piece component. Features of CSTD 1000′″ that correspond to features of CSTD 1000″ are labeled with the same reference numbers followed by three prime symbols (′″). Some features of CSTD 1000′″ that are present in identical or equivalent form in a previously described embodiment(s) will not be described for brevity.

CSTD 1000′″ is similar or identical to CSTD 1000″ in many respects, but has first locking windows 1732′″ that are surrounded by a frame of material on the front side of the device, as shown.

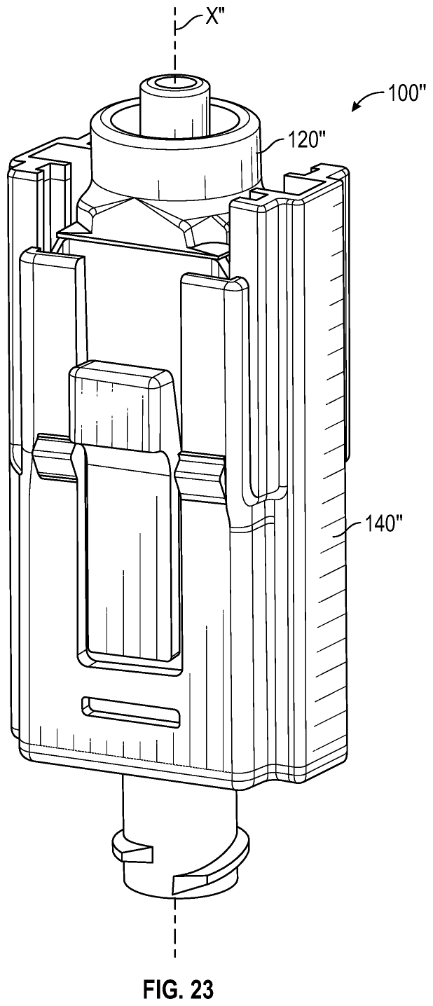

Referring back to FIGS. 23-25, a CSTD 100″ is shown according to another embodiment. CSTD 100″ has a first adaptor 120″ configured to attach to a first fluid reservoir and a second adaptor 140″ configured to attach to a second fluid reservoir. The first adaptor 120″ can be connected to a vial, bag, or patient line, for example. The second adaptor 140″ has a female Luer connector 180″ that can be connected to a syringe or any male Luer connector on a fluid delivery device, such as a pump. Once first adaptor 120″ and second adaptor 140″ are attached to first and second fluid reservoirs, respectively, and interconnected to each other in the coupled state shown in FIG. 23, CSTD 100″ forms a closed fluid passage between the first and second fluid reservoirs. The closed fluid passage is sealed from the outside environment, preventing the inflow of contaminants into the system and the release of hazardous vapors from the system.

CSTD 100″ has a longitudinal axis X extending through the center axis of first adaptor 120″ and center axis of second adaptor 140″ when the first and second adaptors are axially aligned and/or connected. First adaptor 120″ has a generally rectangular body 121″. Second adaptor 140″ has a generally rectangular receptacle or housing 141″. Housing 141″ is adapted to receive body 121″ of first adaptor 120″ in a guided manner so that the two adaptors are axially aligned and centered during mating. Housing 141″ has a hollow interior 143″ and a socket 145″ adapted to receive first adaptor 120″ during mating. Two cut-outs extend on opposite sides of socket 145″ as shown. The cut-outs provide greater access to the interior of housing 141″, as compared to conventional adaptors. The greater access to the interior enables easier disinfection of both sides of the device.

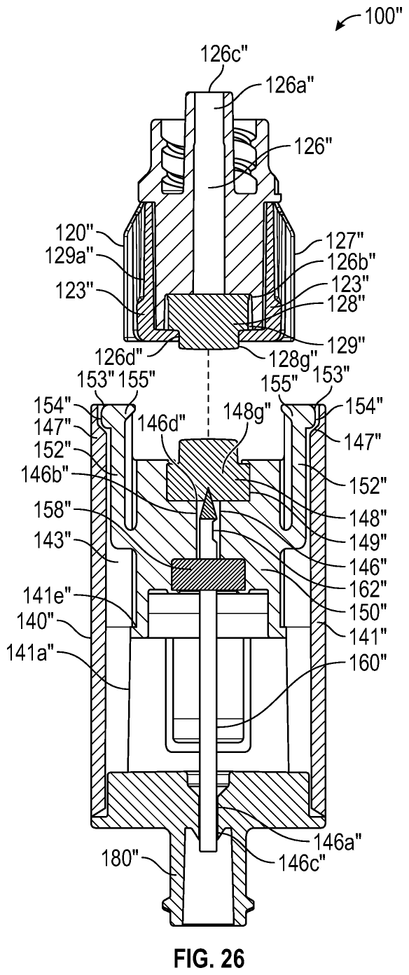

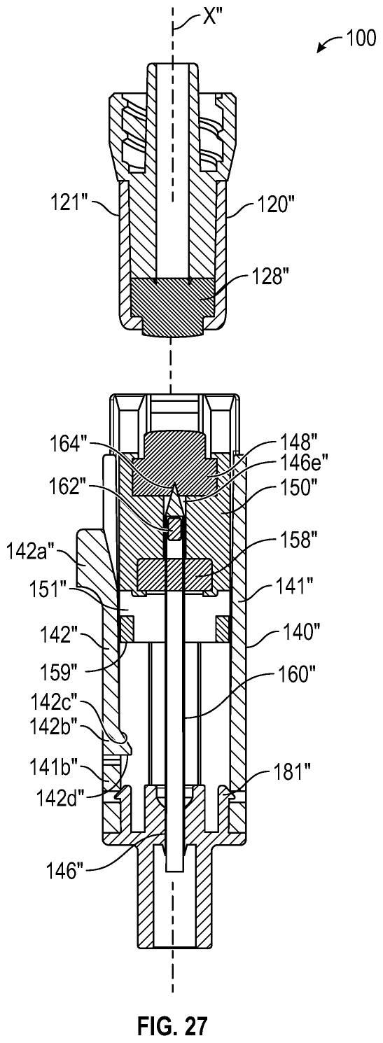

FIGS. 26 and 27 show cross sections of first adaptor 120″ and second adaptor 140″ prior to being coupled together. First adaptor 120″ has a first passage 126″ and a first septum 128″ made of elastomeric material. First passage 126″ has a first passage end 126a″ and a second passage end 126b″ opposite the first passage end. First passage end 126a″ defines a first opening 126c″ and second passage end 126b″ defines a second opening 126d″. First passage 126″ forms a cylindrical chamber or seat 129″ at second passage end 126b″. First septum 128″ is received in seat 129″ to seal the second passage end 126b″. A portion of first septum 128″ extends axially from second opening 126d″, forming a first projection 128g″.

Second adaptor 140″ has a second passage 146″ and a second septum 148″ made of elastomeric material. Second passage 146″ has a first passage end 146a″ and a second passage end 146b″ opposite the first passage end. First passage end 146a″ defines a first opening 146c″ and second passage end 146b″ defines a second opening 146d″. First passage 146″ forms a cylindrical chamber or seat 149″ at the second passage end 146b″. Second septum 148″ is received in seat 149″ to seal the second passage end 146b″. A portion of second septum 148″ extends axially from seat 149″, forming a second projection 148g″. Second projection 148g″ is configured to abut with and be deformed by first projection 128g″, and vice versa, to form a dry break coupling.

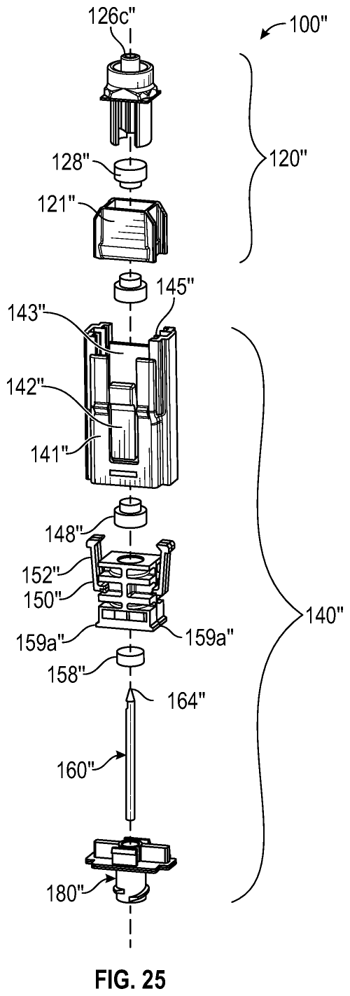

Second septum 148″ is contained in a carrier 150″ that carries the second septum. Carrier 150″ is configured to slide axially inside housing 141″ between a first position and a second position. FIGS. 26 and 27 show carrier 150″ in the first position. Carrier 150″ has two flexible clips 152″ that are made of a resilient flexible material. In the completely relaxed state, clips 152″ extend radially outwardly in a non-parallel arrangement as shown in FIG. 25. When carrier 150″ is assembled inside housing 141″ in the first position, clips 152″ are flexed slightly inwardly so that they are extend parallel to one another as shown in FIG. 26. In this parallel state, clips 152″ are flexed inwardly a small amount in a “relatively relaxed state”, in which some energy is stored in the clips.

Each clip 152″ has a clip end 153″ with a rounded projection 154″ that extends radially outwardly from the clip, and a pointed tip 155″ that extends radially inwardly from the clip. Each rounded projection 154″ rests or bears against a tapered landing 147″ formed in housing 141″ when the carrier is in the first position. Clips 152″ engage landings 147″ to maintain the carrier in the first position, with second septum 148″ located adjacent to socket 145″.

First adaptor 120″ is connected to second adaptor 140″ by inserting body 121″ into socket 145″ until first septum 128″ contacts second septum 148″. Once first septum 128″ abuts second septum 148″, movement of carrier 150″ out of the first position is resisted by the engagement between clips 152″ and landings 147″. This resistance is overcome by applying manual force to first adaptor 120″ in the axial direction toward second septum 148″ until the manual force exceeds the resistance force.

First adaptor 120″ has an outer wall 129a″. Outer wall 129a″ temporarily engages and slides against the pointed tips 155″ of the clips 152″ as first adaptor 120″ moves through socket 145″ into hollow interior 143″. This maintains the clips 152″ in their resting positions against landings 147″ and prevents the clips from flexing further inwardly. Outer wall 129″ tapers inwardly at a transition 123″. In this arrangement, first adaptor 120″ is insertable into second adaptor 140″ so that outer wall 129a″ passes between clips 152″ and slides through pointed tips 155″.

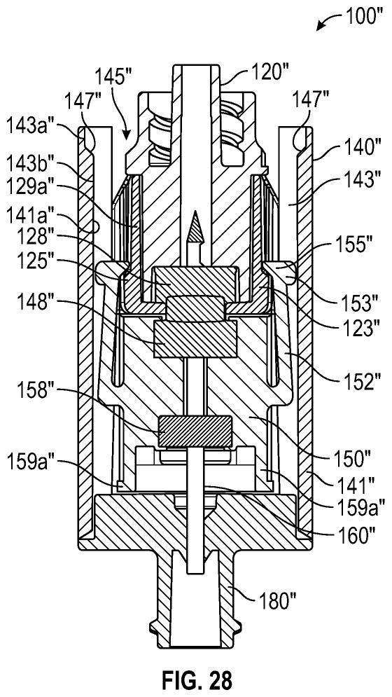

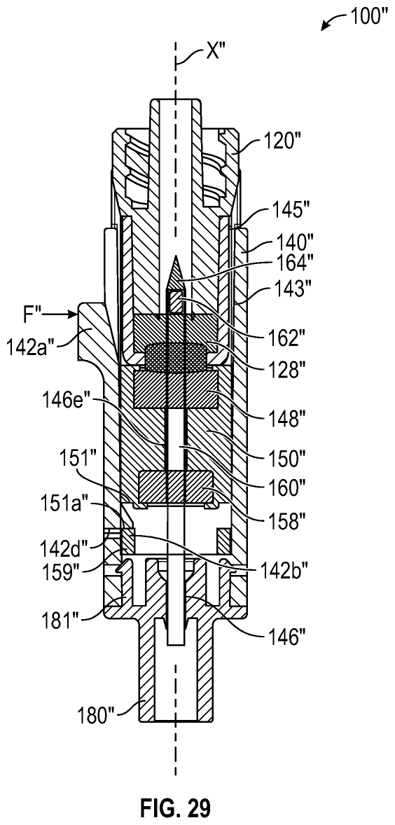

Once outer wall 129a″ passes through pointed tips 155″, the pointed tips slide over the outer wall and reach transition 123″. At this position, outer wall 129a″ no longer prevents inward flexion of clips 152″, as the narrower dimension at transition 123″ provides clearance to allow the clips to flex inward. Hollow interior 143″ of second adaptor 140″ narrows as it extends inwardly from the landings 147″. Therefore, further advancement of first adaptor 120″ into second adaptor 140″ causes clip ends 153″ to flex inwardly so that they no longer bear against landings 147″. At this position, carrier 150″ is free to move from the first position in housing 141″ toward second position. FIGS. 28 and 29 show first adaptor 120″ inserted fully into second adaptor 140″, with carrier 150″ moved to the second position and clips 152″ flexed inwardly.

Hollow interior 143″ of second adaptor 140″ has a wider section 143a″ at socket 145″, which is shown above landings 147″. Hollow interior 143″ transitions to a narrower section 143b″ beneath the landings. As carrier 150″ is advanced from the first position toward the second position, the rounded projections 154″ on clips 152″ slide and bear against the inner wall 141a″ of housing 141′. This abutment between rounded projections 154″ and inner wall 141a″ causes clips 152″ to bend further inwardly into a more flexed state as they enter narrower section 143b″ of hollow interior 143″. The pointed tips 155″ of clips 152″ are pushed inwardly and rest against ledge portions 125″ on outer wall 129a″ of first adaptor 120″.

Second adaptor 140″ houses a needle 160″. Needle 160″ is axially fixed in housing 141″ of second adaptor 140″, while carrier 150″ and second septum 148″ are axially movable relative to the housing. First septum 128″ and second septum 148″ are formed of elastomeric material that can be pierced by needle 160″ as carrier 150″ is pushed toward the second position. Needle 160″ has a side opening 162″ that remains sealed inside carrier 150″ prior to coupling the second adaptor 140″ to the first adaptor 120″. Side opening 162″ is sealed inside a narrow section 146e″ of second passage 146″. Narrow section 146e″ is sealed at a first end by second septum 148″ and sealed at a second end by a third septum 158″.

As first adaptor 120″ is advanced into housing 141″, first septum 128″ pushes against second septum 148″ and moves the second septum and carrier 150″ downwardly in the housing (or toward female Luer connector 180″). Second septum 148″ and carrier 150″ are moved downwardly over needle 160″, which remains fixed in housing 141″. Needle 160″ has a sharp needle tip 164″ configured to penetrate through first septum 128″ and second septum 148″. As carrier 150″ is moved downwardly over needle 160″, needle tip 164″ penetrates through second septum 148″ and immediately enters first septum 128″. During this movement, side opening 162″ of needle 160″ passes through second septum 148″ and immediately into first septum 128″. Therefore, side opening 162″ remains sealed off from other interior areas of CSTD 100″ after emerging from second septum 148″ and passing into first septum 128″.

Side opening 162″ moves through three sealed positions relative to the other components as carrier 150″ moves downwardly over needle 160″. In the first sealed position, shown in FIGS. 26 and 27, side opening 162″ is sealed between second septum 148″ and third septum 158″. In the second sealed position, side opening 162″ is sealed inside second septum 148″. In the third sealed position, side opening 162″ is sealed inside first septum 128″. Once carrier 150″ bottoms out in the receptacle, first septum 128″ is pushed down past side opening 162″, so that side opening 162″ emerges from the first septum and becomes exposed in first passage 126″ of first adaptor 120″. In this state, which is shown in FIGS. 28 and 29, side opening 162″ forms a fluid path of communication between first adaptor 120″, second adaptor 140″, and the reservoirs to which the adaptors are connected.

First projection 128g″ on first septum 128″ abuts second projection 148g″ on second septum 148″ to form a dry break coupling when carrier 150″ reaches the second position. The elastomeric material of first septum 128″ and second septum 148″ compress together, which automatically closes and seals off spaces between needle opening 162″ and interior spaces in CSTD 100″ so that liquid and vapor cannot spill, leak or escape from the device.

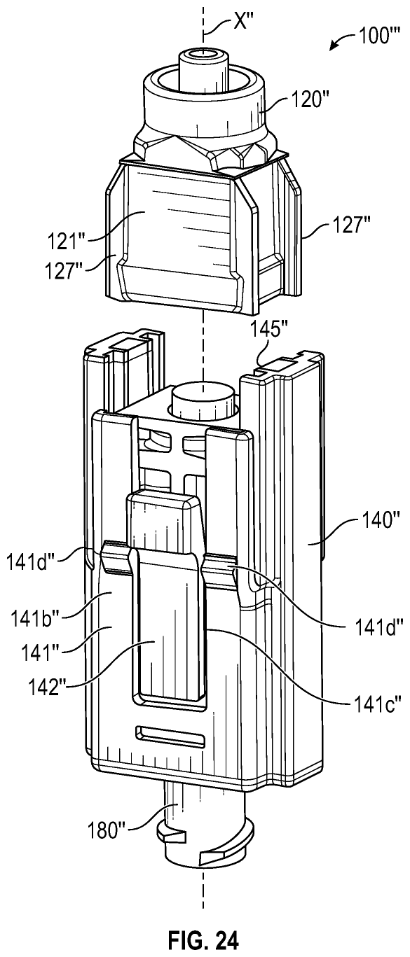

Referring back to FIG. 24, housing 141″ of second adaptor 140″ has a front wall 141b″. Front wall 141b″ defines a longitudinal slot 141c″ that extends from socket 145″ toward female Luer connector 180″. A locking arm 142″ is pivotally mounted in slot 141c″ and extends longitudinally within front wall 141b″. Locking arm 142″ is pivotally connected to front wall 141b″ by a pair of elastic hinges 141d″. In this arrangement, locking arm 142″ is pivotable relative to front wall 141b″ between a locking position shown in FIG. 5 and a release position.

Locking arm 142″ has a first end that forms a button 142a″ and a second end opposite the first end that forms a detent 142b″. Button 142a″ projects radially outwardly from front wall 141b″ when locking arm 142″ is in the locking position. Detent 142b″ projects radially inwardly from front wall 141b″ and into housing 141″ when locking arm 142″ is in the locking position. Detent 142b″ is pivotable in a radially outward direction relative to front wall 141b″ in response to contact with carrier 150″. Detent 142b″ is also pivotable in a radially outward direction relative to front wall 141b″ in response to force applied to button 142a″, as will be explained.

Detent 142b″ has a ramped surface 142c″ that is oriented at an acute angle relative to longitudinal axis X of CSTD 100″. Detent 142b″ also has an abutment surface 142d″ that extends perpendicularly to longitudinal axis X. Carrier 150″ has a locking aperture 151″ above a bottom-most edge 159″. Bottom-most edge 159″ is configured to slidingly engage ramped surface 142c″ as carrier 150″ moves to the second position. Engagement between bottom-most edge 159″ and ramped surface 142c″ causes locking arm 142″ to pivot radially outwardly with energy stored in elastic hinges 141d″. Bottom-most edge 159″ slides over ramped surface 142c″ until detent 142b″ aligns radially with locking aperture 151″. At such time, carrier 150″ bottoms out in housing 141″, reaching the second position shown in FIG. 29. In this position, bottom-most edge 159″ no longer bears against detent 142b″, allowing locking arm 142″ to snap radially inwardly to its relaxed position, with the detent extending into locking aperture 151″. Abutment surface 142d″ engages an abutment edge 151a″ in locking aperture 151″. This engagement prevents carrier 150″ from being moved back to the first position. First adaptor 120″ is interlocked with carrier 150″ by clips 152″, as noted earlier. Therefore, the engagement between abutment surface 142d″ and abutment edge 151a″ also prevents first adaptor 120″ from being withdrawn from second adaptor 140″. This locks CSTD 100″ in a “fluid path open” state in which needle 160″ provides a fluid path between the first adaptor 120″ and second adaptor 140″ and their respective reservoirs.

After fluid is transferred through CSTD 100″, first adaptor 120″ remains locked inside second adaptor 140″ by the engagement between abutment surface 142d″ and abutment edge 151a″, and by the engagement between clips 152″ and ledge portions 125″. First adaptor 120″ can be released from second adaptor 140″ by applying a radially inward force F to button 142a″. The direction of the force F is shown by the arrow in FIG. 29. As button 142a″ is pressed radially inwardly, detent 142b″ pivots radially outwardly and exits locking aperture 151″, with energy stored in hinges 141d″. At this stage, abutment surface 142d″ on detent 142b″ no longer prevents carrier 150″ from being moved from the second position back toward the first position. It also no longer prevents first adaptor 120″ from being moved back toward socket 145″. Therefore, first adaptor 120″ can be removed from hollow interior 143″ of second adaptor 140″ by pressing button 142a″ inwardly to release carrier 150″, and pulling the first adaptor out of the hollow interior and socket 145″.

As first adaptor 120″ is withdrawn from hollow interior 143″, ledge portions 125″ on the first adaptor remain engaged with the undersides of clip ends 153″. This engagement causes carrier 150″ to be towed or pulled back to the first position as first adaptor 120″ is withdrawn from housing 141″. When carrier 150″ reaches the first position, clip ends 153″ exit narrower section 143b″ of hollow interior 143″ and enter wider section 143a″. This causes clip ends 153″ to snap outwardly to the relatively relaxed state shown in FIG. 26. The outward movement of clip ends 153″ releases the clip ends from ledge portions 125″ on first adaptor 120″ Therefore, ledge portions 125″ are freed from clips 152″, allowing first adaptor 120″ to be pulled completely out of housing 141″ and separated from second adaptor 140″. Carrier 150″ is prevented from being pulled out of housing 141″ by flanges 159a″ that extend radially outwardly from bottom-most edge 159″. Flanges 159a″ form stops that engage undercuts 141e″ in inner wall 141a″ of housing 141″, preventing carrier 150″ from being removed from the housing.

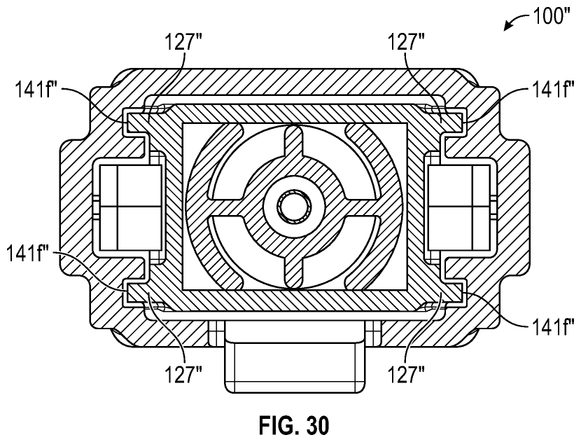

CSTDs according to the present disclosure can include one or more alignment structures that maintain components in proper axial and radial alignment as the components move relative to one another. For example, CSTD 100″ includes longitudinal channels 141f″ along inner wall 141a″ of housing 141″, which are shown in FIG. 30. Channels 141f″ are adapted to receive longitudinally extending rails 127″ on first adaptor 120″ to maintain correct alignment between the first adaptor and housing 141″.

Female Luer connector 180″ is connected with housing 141″ with locking tabs 181″ in a fixed arrangement. In an alternative embodiment, the female Luer connector can be rotatably mounted to the housing with a ratchet mechanism. The ratchet mechanism can be configured to allow a male Luer connector to be screwed onto the female Luer connector in a first direction (e.g. clockwise), but prevent the male Luer connector from being unscrewed from the female Luer connector in a second direction opposite the first direction (e.g. counterclockwise). This provides a safety feature that prevents a user from disconnecting the syringe (or other reservoir) from the second adaptor after liquid has been transferred through the device. The thread and ratchet mechanism can have a variety of configurations, including but not limited to the configurations described in U.S. Pat. Nos. 7,857,805 and 5,328,474, the contents of both patents being incorporated by reference herein in their entireties.

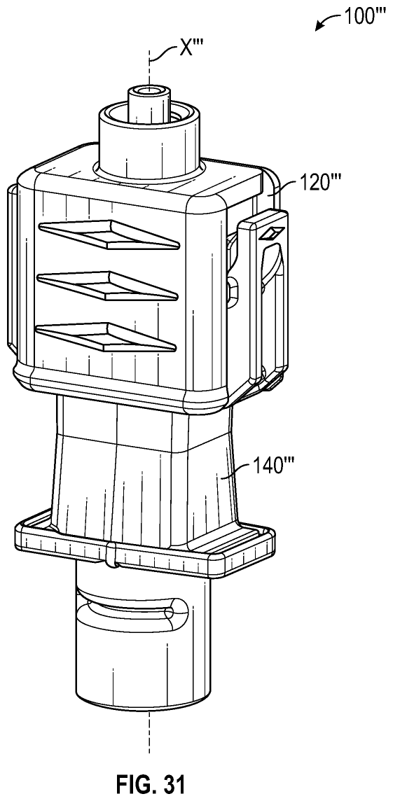

Referring to FIGS. 31-33, a CSTD 100′″ is shown according to another embodiment. CSTD 100′″ has a first adaptor 120′″ configured to attach to a first fluid reservoir and a second adaptor 140′″ configured to attach to a second fluid reservoir. The first adaptor 120′″ can be connected to a vial, bag, or patient line, for example. The second adaptor 140′″ has a female Luer connector 180′″ that can be connected to a syringe or any male Luer connector on a fluid delivery device, such as a pump. Once first adaptor 120′″ and second adaptor 140′″ are attached to first and second fluid reservoirs, respectively, and interconnected to each other in the coupled state shown in FIG. 31, CSTD 100′″ forms a closed fluid passage between the first and second fluid reservoirs. The closed fluid passage is sealed from the outside environment, preventing the inflow of contaminants into the system and the release of hazardous vapors from the system.

CSTD 100′″ has a longitudinal axis X extending through the center axis of first adaptor 120′″ and center axis of second adaptor 140′″ when the first and second adaptors are axially aligned and/or connected. First adaptor 120′″ has a generally rectangular body 121′″. Second adaptor 140′″ has a generally rectangular receptacle or housing 141′″. Body 121′″ of first adaptor 120′″ is adapted to receive housing 141′″ in a guided manner so that the two adaptors are axially aligned and centered during mating. Housing 141′″ has a hollow interior 143′″ and a socket 145′″ adapted to receive an interior portion of first adaptor 120′″ during mating, as will be explained. Two cut-outs extend on opposite sides of socket 145′″ as shown. The cut-outs provide greater access to the interior of housing 141′″, as compared to conventional adaptors. The greater access to the interior enables easier disinfection of both sides of the device.

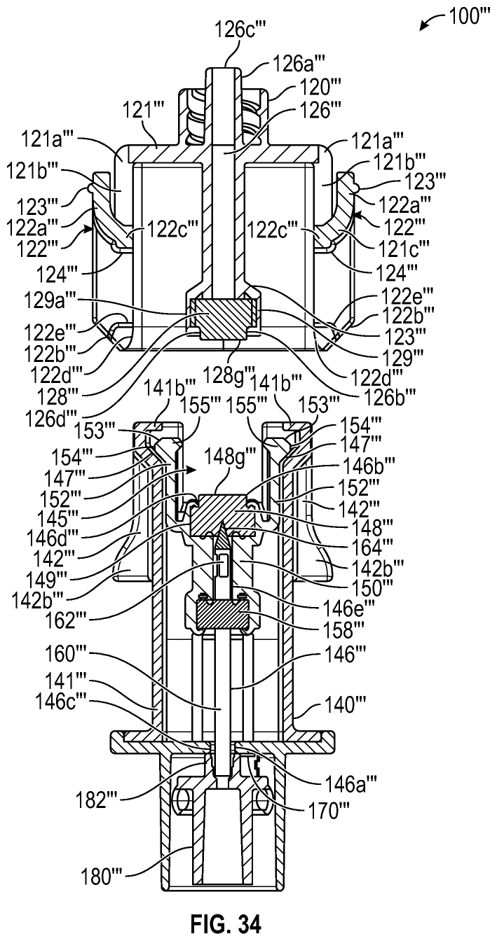

FIG. 34 shows cross sections of first adaptor 120′″ and second adaptor 140′″ prior to being coupled together. First adaptor 120′″ has a first passage 126′″ and a first septum 128′″ made of elastomeric material. First passage 126′″ has a first passage end 126a′″ and a second passage end 126b′″ opposite the first passage end. First passage end 126a′″ defines a first opening 126c′″ and second passage end 126b′″ defines a second opening 126d′″. First passage 126′″ forms a cylindrical chamber or seat 129′″ at second passage end 126b′″. First septum 128′″ is received in seat 129′″ to seal the second passage end 126b′″. A portion of first septum 128′″ extends axially from second opening 126d′″, forming a first projection 128g′″.

Second adaptor 140′″ has a second passage 146′″ and a second septum 148′″ made of elastomeric material. Second passage 146′″ has a first passage end 146a′″ and a second passage end 146b′″ opposite the first passage end. First passage end 146a′″ defines a first opening 146c′″ and second passage end 146b′″ defines a second opening 146d″. First passage 146′″ forms a cylindrical chamber or seat 149′″ at the second passage end 146b′″. Second septum 148′″ is received in seat 149′″ to seal the second passage end 146b′″. A portion of second septum 148′″ extends axially from seat 149′″, forming a second projection 148g′″. Second projection 148g′″ is configured to abut with and be deformed by first projection 128g′″, and vice versa, to form a dry break coupling.

Second septum 148′″ is contained in a carrier 150′″ that carries the second septum. Carrier 150′″ is configured to slide axially inside housing 141′″ between a first position and a second position. FIG. 34 shows carrier 150′″ in the first position. Carrier 150′″ has two flexible clips 152′″ that are made of a resilient flexible material. In a completely relaxed state, clips 152′″ extend parallel to one another.

Each clip 152′″ has a clip end 153′″ with a rounded projection 154′″ that extends radially outwardly from the clip, and a pointed tip 155′″ that extends radially inwardly from the clip. Each rounded projection 154′″ rests or bears against a tapered landing 147′″ formed in housing 141′″ when the carrier is in the first position. Clips 152′″ engage landings 147′″ to maintain the carrier in the first position, with second septum 148′″ located adjacent to socket 145′″.

First adaptor 120′″ is connected to second adaptor 140′″ by inserting housing 141′″ of the second adaptor into body 121′″ of the first adaptor. Housing 141′″ is inserted into body 121′″ until first septum 128′″ contacts second septum 148′″. Once first septum 128′″ abuts second septum 148′″, movement of carrier 150′″ out of the first position is resisted by the engagement between clips 152′″ and landings 147′″. This resistance is overcome by applying manual force to first adaptor 120′″ in the axial direction toward second septum 148′″ until the manual force exceeds the resistance force.

First adaptor 120′″ has an outer wall 129a′″. Outer wall 129a′″ temporarily engages and slides against the pointed tips 155′″ of the clips 152′″ as the outer wall moves through socket 145′″ into hollow interior 143′″. This maintains the clips 152′″ in their resting positions against landings 147′″ and prevents the clips from flexing further inwardly. In this arrangement, outer wall 129a′″ and first septum 128′″ are insertable into hollow interior 143′″ of second adaptor 140′″ and pass through pointed tips 155′″.

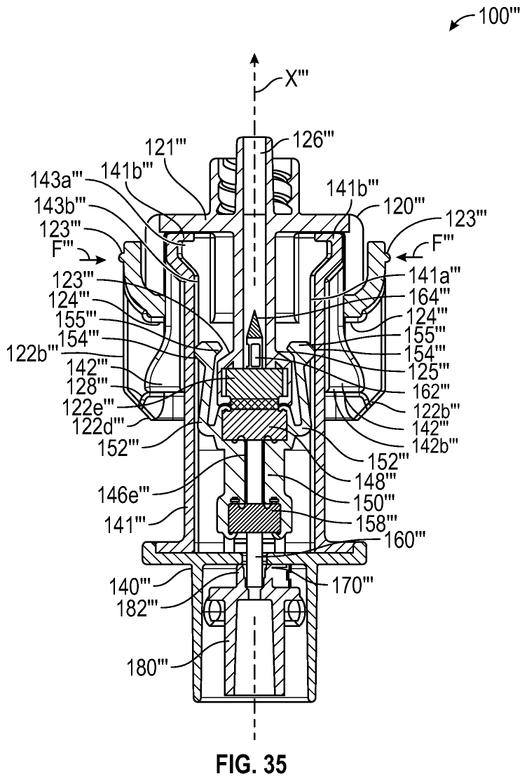

Once outer wall 129a′″ passes through pointed tips 155′″, the pointed tips slide over the outer wall and reach a transition 123′″ where the outer wall tapers radially inwardly. When pointed tips 155′″ reach this position, outer wall 129a′″ no longer prevents inward flexion of clips 152′″, as the narrower dimension at transition 123′″ provides clearance to allow the clips to flex inward. Hollow interior 143′″ of second adaptor 140′″ narrows as it extends inwardly from the landings 147′″. Therefore, further advancement of outer wall 129a′″ and first septum 128′″ into housing 141′″ causes clip ends 153′″ to flex inwardly so that they no longer bear against landings 147′″. At this position, carrier 150′″ is free to move from the first position in housing 141′″ toward the second position. FIG. 35 shows outer wall 129a′″ and first septum 128′″ inserted fully into housing 141′″, with carrier 150′″ moved to the second position and clips 152′″ flexed inwardly.

Referring to FIGS. 34 and 35, hollow interior 143′″ of second adaptor 140′″ has a wider section 143a′″ at socket 145′″, which is shown above landings 147′″. Hollow interior 143′″ transitions to a narrower section 143b′″ beneath the landings. As carrier 150′″ is advanced from the first position toward the second position, the rounded projections 154′″ on clips 152′″ slide and bear against the inner wall 141a′″ of housing 141′″. This abutment between rounded projections 154′″ and inner wall 141a′″ causes clips 152′″ to bend inwardly into a flexed state with stored energy as they enter narrower section 143b′″. The pointed tips 155′″ of clips 152′″ are pushed inwardly and rest against ledge portions 125′″ on outer wall 129a′″ of first adaptor 120′″.

Second adaptor 140′″ houses a needle 160′″. Needle 160′″ is axially fixed in housing 141′″ of second adaptor 140′″, while carrier 150′″ and second septum 148′″ are axially movable relative to the housing. First septum 128′″ and second septum 148′″ are formed of elastomeric material that can be pierced by needle 160′″ as carrier 150′″ is pushed toward the second position. Needle 160′″ has a side opening 162′″ that remains sealed inside carrier 150′″ prior to coupling second adaptor 140′″ to first adaptor 120′″. Side opening 162′″ is sealed inside a narrow section 146e′″ of second passage 146′″. Narrow section 146e′″ is sealed at a first end by second septum 148′″ and sealed at a second end by a third septum 158″.

As outer wall 129a′″ and first septum 128′″ advance into housing 141′″, the first septum pushes against second septum 148′″ and moves the second septum and carrier 150′″ downwardly in the housing (or toward female Luer connector 180′″). Second septum 148′″ and carrier 150′″ are moved downwardly over needle 160′″, which remains fixed in housing 141′″. Needle 160′″ has a sharp needle tip 164′″ configured to penetrate through first septum 128′″ and second septum 148′″. As carrier 150′″ is moved downwardly over needle 160′″, needle tip 164′″ penetrates through second septum 148′″ and immediately enters first septum 128″. During this movement, side opening 162′″ of needle 160′″ passes through second septum 148′″ and immediately into first septum 128′″. Therefore, side opening 162′″ remains sealed off from other interior areas of CSTD 100′″ after emerging from second septum 148′″ and passing into first septum 128′″.

Side opening 162′″ moves through three sealed positions relative to the other components as carrier 150′″ moves downwardly over needle 160′″. In the first sealed position, shown in FIG. 34, side opening 162′″ is sealed between second septum 148′″ and third septum 158′″. In the second sealed position, side opening 162′″ is sealed inside second septum 148′″. In the third sealed position, side opening 162′″ is sealed inside first septum 128′″. Once carrier 150′″ bottoms out in the receptacle, first septum 128′″ is pushed down past side opening 162′″, so that side opening 162′″ emerges from the first septum and becomes exposed in first passage 126′″ of first adaptor 120′″. In this state, which is shown in FIG. 35, side opening 162′″ forms a fluid path of communication between first adaptor 120′″, second adaptor 140′″, and the reservoirs to which the adaptors are connected.

First projection 128g′″ on first septum 128′″ abuts second projection 148g′″ on second septum 148′″ to form a dry break coupling when carrier 150′″ reaches the second position. The elastomeric material of first septum 128′″ and second septum 148′″ compress together, which automatically closes and seals off spaces between needle opening 162′″ and interior spaces in CSTD 100′″ so that liquid and vapor cannot spill, leak or escape from the device.

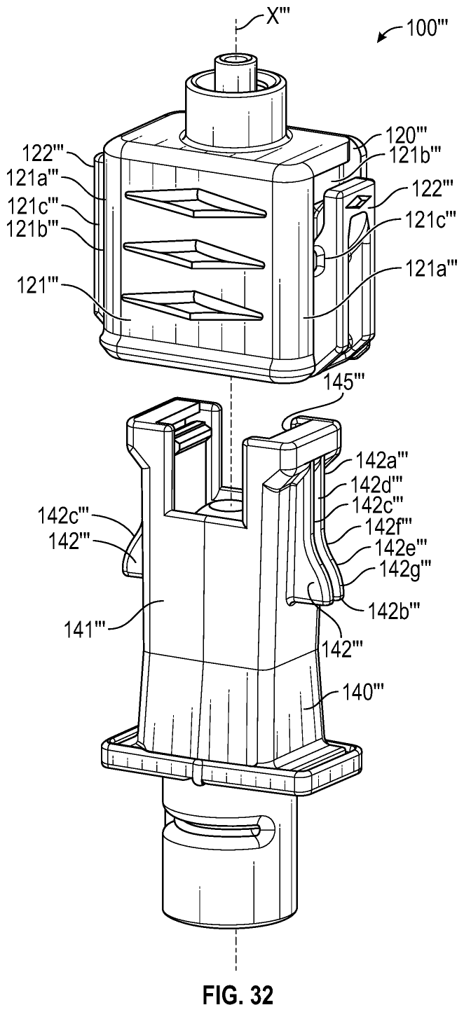

Referring back to FIG. 32, body 121′″ of first adaptor 120′″ has a pair of side walls 121a′″. Each side wall 121a′″ defines a longitudinal slot 121b′″. A locking arm 122′″ is pivotally mounted in each slot 121b′″ and extends longitudinally within each side wall 121a′″. Each locking arm 122′″ is pivotally connected to its respective side wall 121a′″ by a pair of elastic hinges 121c′″. In this arrangement, each locking arm 122′″ is pivotable relative to its respective side wall 121a′″ between a locking position shown in FIG. 35 and a release position. Each locking arm 122′″ has a first end 122a′″ that forms a button 123′″ and a second end 122b′″ opposite the first end that defines a locking aperture 124′″. Each locking aperture 124′″ is configured to cooperatively engage a section of second adaptor 140′″ to releasably lock first adaptor 120′″ onto the second adaptor.

Referring again to FIG. 32, second adaptor 140′″ has two locking ramps 142′″ that project radially outwardly from the exterior of housing 141′″. Each locking ramp 142′″ is configured to project through one of the locking apertures 124′″ on first adaptor 120′″ to releasably lock second adaptor 140′″ to the first adaptor. Each locking ramp 142′″ has a leading end 142a′″, a trailing end 142b′″, and a ramped surface 142c′″ extending between the leading end and trailing end. Ramped surface 142c′″ has a straight section 142d′″ adjacent leading end 142a′″ that is parallel to longitudinal axis X. Ramped surface 142′″ also has a curved section 142e′″ extending between straight section 142d′″ and trailing end 142b′″. Curved section 142e′″ has a compound curvature which defines a concave portion 142f′″ and a convex portion 142g′″.

Referring to FIG. 34, each locking arm 122′″ has a sliding face 122c′″ that faces radially inwardly. Each sliding face 122c′″ has a rounded convex bearing surface 122d′″ at the second end 122b′″ of the locking arm. Bearing surfaces 122d′″ are configured to slidingly engage the curved sections 142e′″ of ramped surfaces 142c′″ after first septum 128′″ begins moving the second septum 148′″ and carrier 150′″ toward the second position. When housing 141′″ initially enters body 121′″ of first adaptor 120′″, bearing surfaces 122d′″ on locking arms 122′″ slide along straight sections 142d′″ of locking ramps 142′″. Locking arms 122′″ remain in the same general orientation during this sliding engagement. As housing 141′″ advances further into body 121′″, bearing surfaces 122d′″ eventually reach curved sections 142e′″ on ramped surfaces 142c′″ and slide along the curved sections. The inclined geometries of curved sections 142e′″ exert radially outward forces on bearing surfaces 122d′″, causing the second ends 122b′″ of locking arms 122′″ to pivot radially outwardly. As locking arms 122′″ pivot through side walls 121a′″, energy is stored in elastic hinges 121c′″.

Housing 141′″ is advanced into body 121′″ until locking ramps 142′″ align in a radial direction with locking apertures 124″. At such time, the trailing ends 142b′″ of locking ramps 142′″ pass through bearing surfaces 122d′″. Bearing surfaces 122d′″ no longer bear against locking ramps 142′″, allowing locking arms 122′″ to release the energy stored in elastic hinges 121c′″ and snap radially inwardly back to a more relaxed state in their locking positions. Locking arms 122′″ return to the locking positions with locking ramps 142′″ entrapped inside locking apertures 124′″, as shown in FIG. 35. This occurs at the same moment or approximately the same moment when side opening 162′″ of needle 160′″ fully emerges from first septum 128′″ and enters first passage 126′″ in first adaptor 120′″.

When locking arms 122′″ snap inwardly to the locking positions, trailing ends 142b′″ of locking ramps 142′″ are positioned adjacent abutment surfaces 122e′″ inside locking apertures 124″. Abutment surfaces 122e′″ form axial stops or obstructions that engage trailing ends 122b′″ of locking ramps. As such, abutment surfaces 122e′″ prevent housing 141′″ from being reversed out of body 121′″ when the locking arms 142′″ are in the locking positions. This locks CSTD 100′″ in a “fluid path open” state in which needle 160′″ provides a fluid path between the first adaptor 120′″ and second adaptor 140′″ and their respective reservoirs.

After fluid is transferred through CSTD 100′″, housing 141′″ of second adaptor remains locked inside first adaptor 120′″ by the engagement between the abutment surfaces 122e′″ and the trailing ends 142b′″ of locking ramps 142′″. Housing 141′″ can be released from first adaptor 120′″ by applying radially inward forces F to each button 123′″. The directions of forces F′″ are shown by the arrows in FIG. 35. As buttons 123′″ are pressed radially inwardly, second ends 122b′″ of locking arms 122′″ pivot radially outwardly with energy once again stored in hinges 121c′″. Buttons 123′″ are pressed radially inwardly until abutment surfaces 122e′″ pivot outwardly enough to clear the trailing ends 142b′″ of locking ramps 142′″. At this stage, locking ramps 142b′″ are no longer enclosed by locking apertures 124′″, and abutment surfaces 122e′″ no longer obstruct axial movement of locking ramps 142′″ relative to first adaptor 120′″. Therefore, second adaptor 140′″ can be disconnected from first adaptor 120′″ by pressing buttons 123′″ radially inwardly to release locking ramps 142′″, and pulling housing 141′″ out of the first adaptor.

As housing 141′″ is withdrawn from first adaptor 120′″, ledge portions 125′″ on the first adaptor remain engaged with the undersides of clip ends 153′″, which are still bent inwardly. This engagement causes carrier 150′″ to be towed or pulled back to the first position as housing 141′″ is withdrawn from first adaptor 120′″. When carrier 150′″ reaches the first position, clip ends 153′″ exit narrower section 143b′″ of hollow interior 143′″ and enter wider section 143a′″. This causes clip ends 153′″ to snap outwardly to the relatively relaxed state shown in FIG. 34. The outward movement of clip ends 153′″ releases the clip ends from ledge portions 125′″ on first adaptor 120′″. Therefore, ledge portions 125′″ are freed from clips 152′″, allowing outer wall 129a′″ and first septum 128′″ to be separated from carrier 150′″ and pulled out of housing 141′″.

Carrier 150′″ is prevented from being pulled out of housing 141′″ by a pair of end walls 141b′″ that extend radially inwardly. End walls 141b′″ define a narrowed opening 145a′″ at socket 145′″. The width of opening 145a′″ is smaller than the spacing between rounded projections 154′″ when carrier 150′″ is in the first position. As such, end walls 141b′″ form stops that engage rounded projections 154′″ to prevent carrier 150′″ from being removed from housing 141′″.

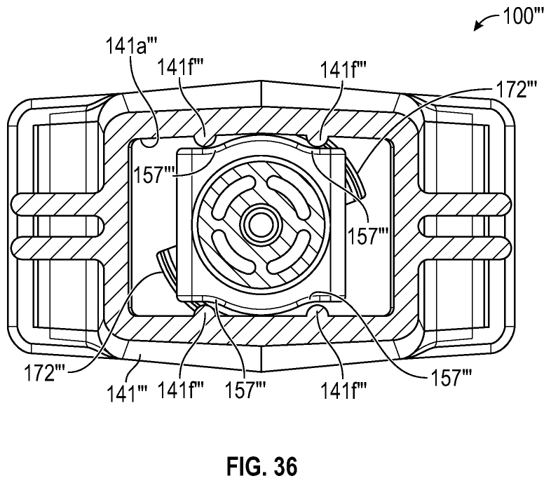

CSTDs according to the present disclosure can include one or more alignment structures that maintain components in proper axial and radial alignment as the components move relative to one another. For example, CSTD 100′″ includes longitudinal ribs 141f′″ along inner wall 141a′″ of housing 141′″, as shown in FIG. 36. Ribs 141f′″ are configured to abut longitudinally extending recesses or grooves 157′″ on carrier 150′″ to maintain correct alignment between the carrier and housing 141′″.

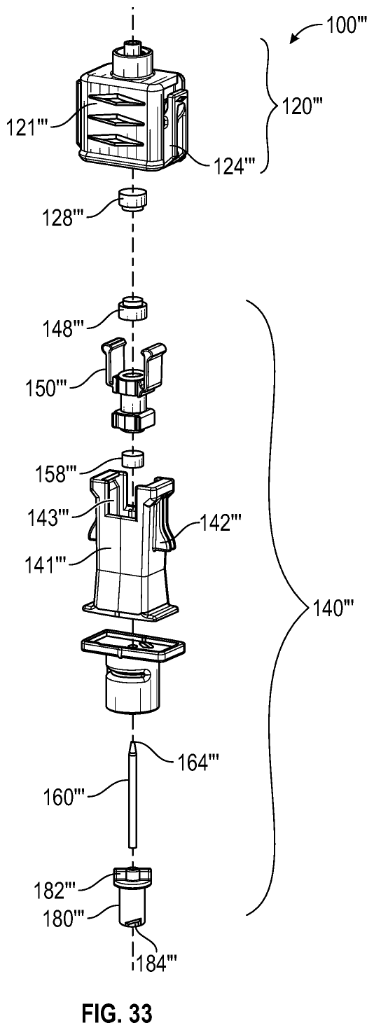

Female Luer connector 180′″ is rotatably mounted to housing 141′″ with a ratchet mechanism 170′″, elements of which are shown in FIGS. 33-36. A flange 182′″ at a top end of female Luer connector 180′″ is configured to slidingly engage ramped surfaces 172′″ of ratchet mechanism 170′″ as the female Luer connector 180′″ rotates relative to housing 141′″. Ramped surfaces 172′″ allow flange 182′″, and consequently female Luer connector 180′″, to rotate in one direction but not the opposite direction. Female Luer connector 180′″ has a thread 184′″ at a bottom end configured to mate with a thread on a male Luer connector. In this arrangement, ratchet mechanism 170′″ is configured to allow a male Luer connector to be screwed onto female Luer connector 180′″ in a first direction (e.g. clockwise), but prevent the male Luer connector from being unscrewed from the female Luer connector in a second direction opposite the first direction (e.g. counterclockwise). This provides a safety feature that prevents a user from disconnecting the syringe (or other reservoir) from the second adaptor after liquid has been transferred through the device. The thread and ratchet mechanism can have a variety of configurations, including but not limited to the configurations described in U.S. Pat. Nos. 7,857,805 and 5,328,474, the contents of both patents being incorporated by reference herein in their entireties.

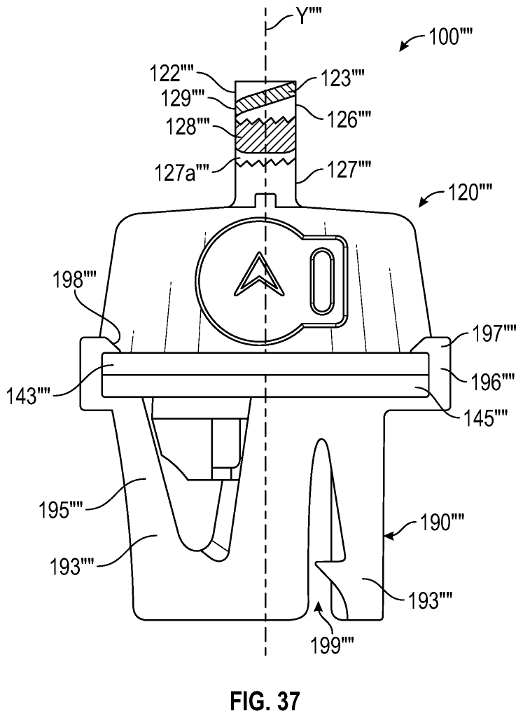

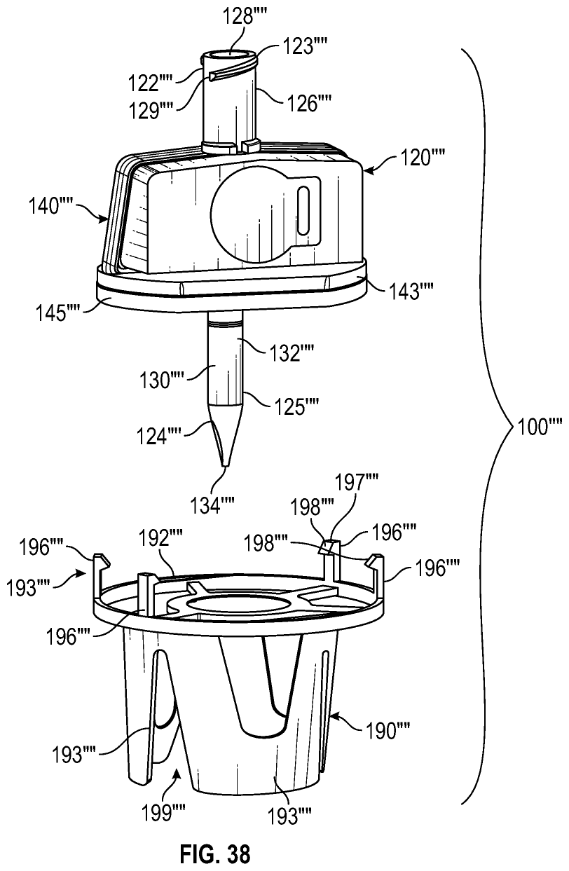

Referring to FIGS. 37 and 38, a CSTD 100″″ is shown according to another embodiment. CSTD 100″″ has first and second adaptors that are configured to connect a vial with another fluid reservoir. This type of CSTD is referred to herein as a “vial adaptor”. CSTD 100″″ includes a first adaptor referred to as a “vial spike” 120″″ and a second adaptor referred to a base or “vial clip” 190″″. CSTD 100″″ has a longitudinal axis Y″″ which passes through center portions of vial spike 120″″ and vial clip 190″″. Vial spike 120″″ has a proximal end 122″″ forming a first connector 123″″ configured to fluidly connect to a first fluid reservoir, such as a syringe. Vial spike 120″″ also has a distal end 124″″ forming a second connector 125″″ configured to fluidly connect to a second fluid reservoir, such as a vial. Once vial spike 120″″ is connected in fluid communication with first and second fluid reservoirs, respectively, the vial spike forms a closed fluid passage between the first and second fluid reservoirs. This closed fluid passage, which is also referred to herein as a “transfer line”, allows liquid to be transferred between the first and second fluid reservoirs in a sealed manner that prevents release of hazardous drugs into the environment.

Vial spikes according to the present disclosure can have a variety of fluid connectors at the proximal and distal ends. In the present embodiment, first connector 123″″ is a dry break coupling 126″″ that prevents liquid or vapor from being released from the CSTD during coupling or decoupling of the vial spike from the first fluid reservoir. Dry break coupling 126″″ has a cylindrical body 127″″ defining a fluid passage 127a″″. A septum 128″″ is mounted in fluid passage 127a″″ inside cylindrical body 127″″. Cylindrical body 127″″ has an external thread 129″″ configured to mate with an internal thread on syringe or other fluid reservoir.

Second connector 125″″ is a spike connector 130″″ having a cylindrical profile 132″″ and pointed tip 134″″. Spike connector 130″″ has separate fluid passages that convey liquid and gas through the CSTD as will be explained.

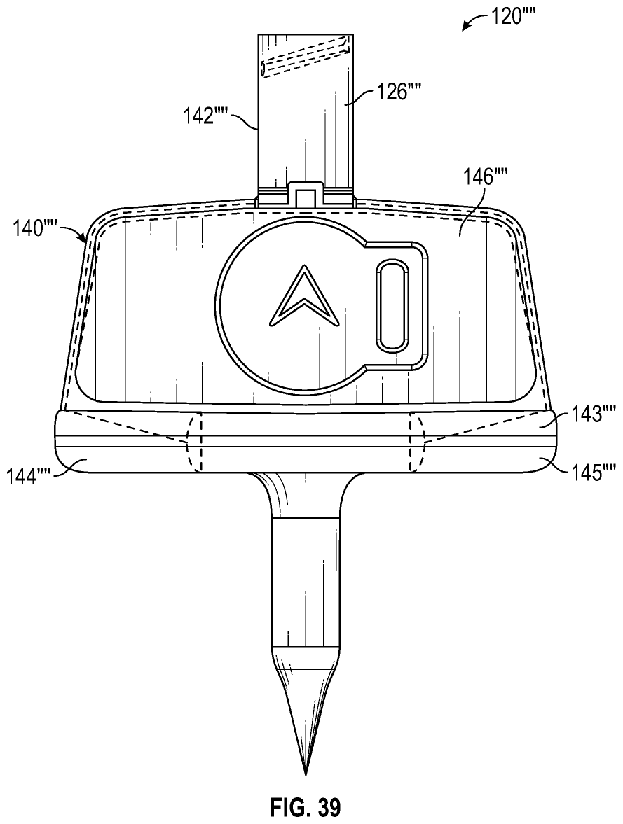

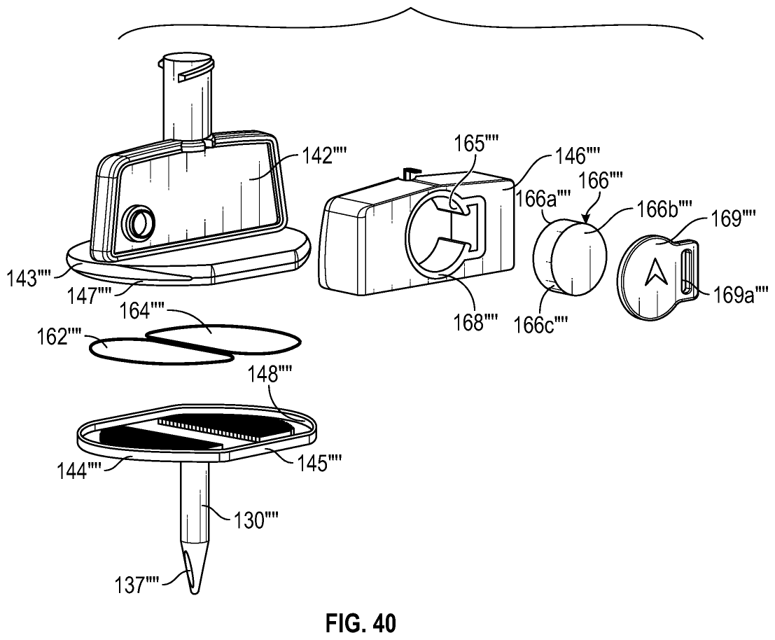

Referring to FIGS. 39-41, vial spike 120″″ has a three-part housing 140″″ that forms passageways for liquid and gas flowing within CSTD 100″″. Housing 140″″ includes a first housing portion 142″″ adjacent to and fluidly connected with dry break coupling 126″″ Housing 140″″ also includes a second housing portion 144″″ adjacent to and fluidly connected with spike connector 130″″. Moreover, housing 140″″ includes a third housing portion or casing 146″″ which attaches to one side of first housing portion 142″.

First housing portion 142″″ has a first cover piece 143″″ opposite dry break coupling 126″″. Second housing portion 144″″ has second cover piece 145″″ opposite spike connector 130″″. Second cover piece 145″″ is configured to connect with first cover piece 143″″ to join the first housing portion 142″″ and second housing portion 144″″ together. First cover piece 143″″ and second cover piece 145″″ are shaped so as to as to form a narrow gap, void, or space 141″″ between first housing portion 142″″ and second housing portion 144″″. A ring-shaped lip portion 147″″ extends around the periphery of first cover piece 143″″ and projects from first housing portion 142″″. A ring-shaped wall portion 148″″ extends around the periphery of second cover piece 145″″, forming a receptacle slightly larger in size than the ring-shaped lip portion 147″″. Ring-shaped wall portion 148″″ is adapted to receive ring-shaped lip portion 147″″ to join first housing portion 142″″ to second housing portion 144′″ in a mated and sealed arrangement.

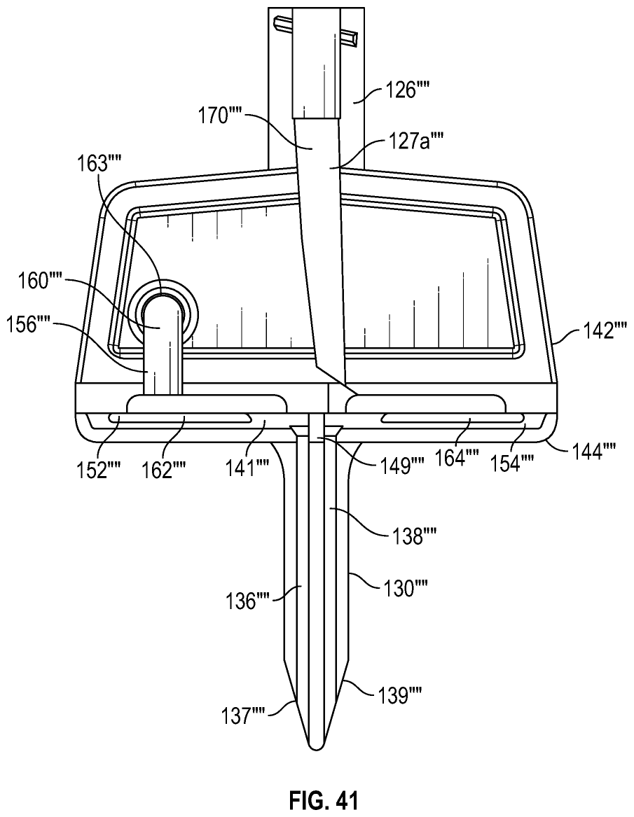

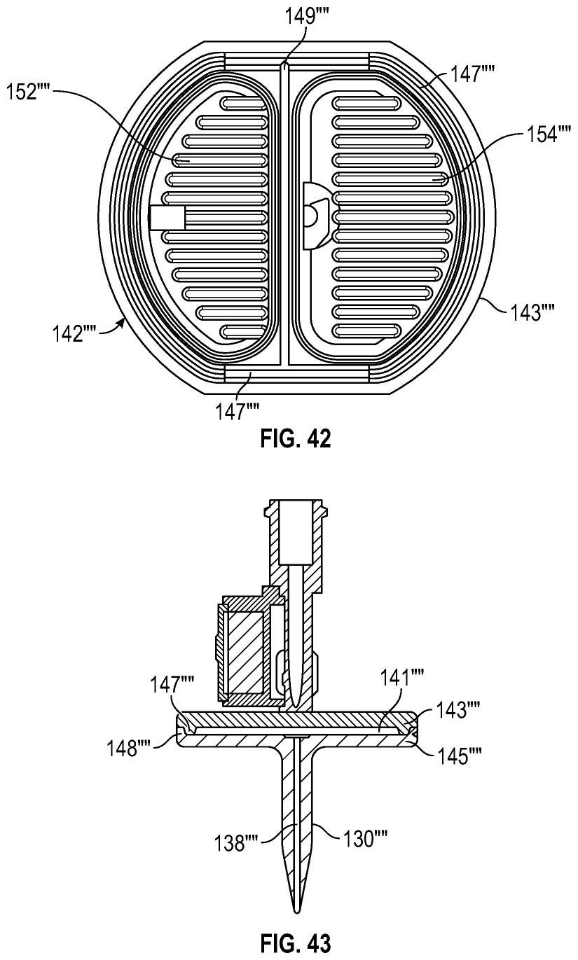

Referring to FIGS. 41-43, first cover piece 143″″ has a divider wall 149″″ that spans the center of ring-shaped lip portion 147″″ as shown. When first cover piece 143″″ is mated with second cover piece 145″″, ring-shaped wall portion 148″″ and divider wall 149″″ create two separate chambers inside the first and second cover pieces. A first chamber 152″″ is formed on a first side of divider wall 149″″, and a second chamber 154″″ is formed on a second side of the divider wall opposite the first side.

First cover piece 143″″ and second cover piece 145″″ are connected to one another so that they fully enclose first chamber 152″″ and second chamber 154″″ and seal the chambers in a fluid tight manner from the exterior of vial spike 120″″. In addition, first cover piece 143″″ and second cover piece 145″″ are connected to one another to seal first chamber 152″″ in a fluid tight manner from second chamber 154″″, and vice versa. In this regard, first cover piece 143″″ can be joined to second cover piece 145″″ using any suitable means that seals the chambers from the exterior of vial spike 120″″, and seals the chambers from one another. Suitable means for establishing such a sealed arrangement include welding techniques such as ultrasonic welding, hot plate welding and laser welding. Suitable means for establishing a sealed arrangement also include over molding and gluing.

Spike connector 130″″ has a first passage 136″″ and a second passage 138″″ extending parallel to the first passage. When vial spike 120″″ is fully assembled, first passage 136″″ fluidly connects with first chamber 152″″ but not with second chamber 154″″. In addition, second passage 138″″ fluidly connects to second chamber 154″″ but not first chamber 152″″. First passage 136″″ extends the entire length of spike connector 130″″ and exits pointed tip 134″″ where it forms a first opening 137″″. Second fluid passage 138″″ also extends the entire length of spike connector 130″″ and exits pointed tip 134″″ where it forms a second opening 139″″. First chamber 152″″ fluidly connects to a vent passage 156″″ in first housing portion 142″″. Second chamber 154″″ fluidly connects to fluid passage 127a″″ of dry break coupling 126″″. In this arrangement, first passage 136″″ forms part of a vent line 160″″ that equalizes pressure in CSTD 100″″ with outside atmospheric pressure, as will be explained, while second passage 138″″ forms part of a transfer line 170″″ for transferring liquid between the first fluid reservoir and second fluid reservoir. Vent line 160″″ and transfer line 170″″ are sealed from one another within CSTD 100″″, so that gas carried in vent line 160″″ does not enter transfer line 170″″, and liquid carried in the transfer line does not enter the vent line.

Each chamber in CSTD 100″″ is configured to contain a filter material or filter component dedicated to each respective line. The chambers need not contain any filters, however. In the present example, first chamber 152″″ contains a first filter component in the form of a hydrophobic filter 162″″, and second chamber 154″″ contains a second filter component in the form of a particle filter 164″″ arranged in parallel with the hydrophobic filter in a coplanar arrangement. Hydrophobic filter 152″″ is optional, and particle filter 164″″ is optional, depending on the application.

Vent line 160″″ allows air from the atmosphere to enter CSTD 100″″. Hydrophobic filter 162″″ is configured to allow air from the atmosphere to pass through the hydrophobic filter, while not allowing liquids and aerosols in the air to pass through the hydrophobic filter. This prevents contaminants from the atmosphere from passing through the first chamber 152″ through the first passage 136″″ and into the second fluid reservoir. It also prevents or substantially prevents liquids and aerosols from the second fluid reservoir from going beyond the first chamber 152″″. Hydrophobic filter 162″″ can have any suitable pore size, including but not limited to a pore size of 0.2 microns.

Particle filter 164″″ is configured to allow liquid to pass through the particle filter, but not allow small particles from passing through the particle filter. This prevents particles in the first fluid reservoir from transferring to the second fluid reservoir, and vice versa. Particle filters according to the present disclosure can be comprised of any suitable material for filtering particulates, including but not limited to membranes formed of acrylic copolymer, polyether sulfone or polyvinylidene fluoride.

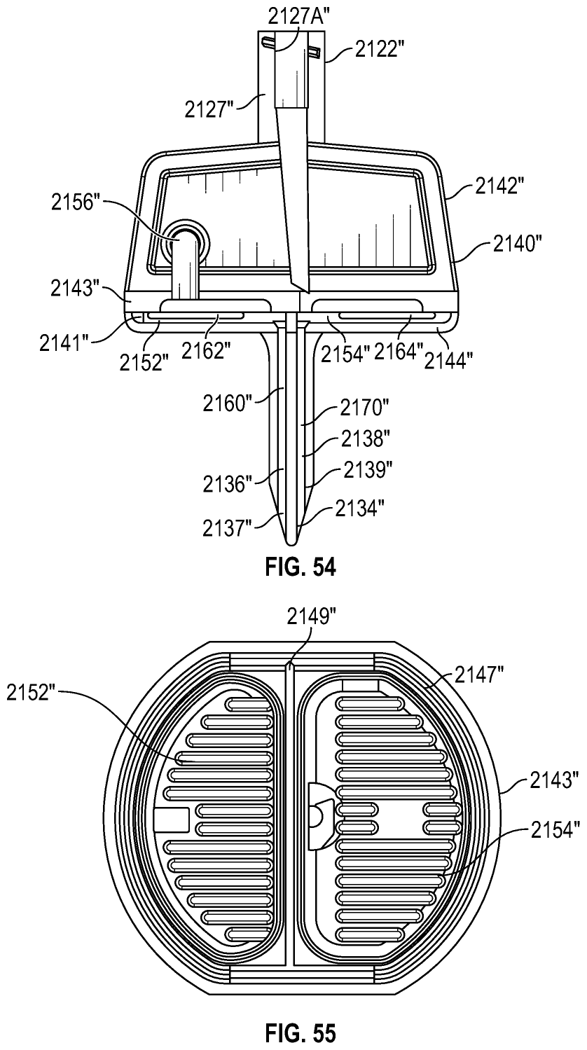

Vent line 160″″ extends from first passage 136″″, through first chamber 152′″ and into first housing portion 142″″. Vent line 160″″ exits first housing portion 142″″ through a side port 164″″ and into third housing portion 146″″. Third housing portion 146″″ has a hollow shell structure that connects to one side of first housing portion 142″″ and over side port 164″″. Referring to FIG. 54, third housing portion 146″″ is shown transparent to illustrate the interior and the directions of gas flow through the third housing portion.

The interior of third housing portion 146″″ provides a flow passage 163″″ that forms a section of vent line 160″″. Third housing portion 146″″ also includes a filter housing 165″″ Filter housing 165″″ houses a third filter component in the form of an activated carbon filter 166″″. Third housing portion 146″″ further includes an outlet 168″″ adjacent to activated carbon filter 166″″. Outlet 168′″ is covered by cap 169″″ to retain and protect the activated carbon filter 166″″ inside third housing portion 146″″. Cap 169″″ has a small outlet aperture 169a″″ that connects with the atmosphere. Flow passage 163″″ extends from side port 164″″ through activated carbon filter 166″″ and exits third housing portion 146″″ through outlet aperture 169a″″.

In this arrangement, gas under positive pressure in the second fluid reservoir can exhaust out of CSTD 100″″ by flowing through flow passage 163″″ and activated carbon filter 166″″ and then exiting through outlet aperture 169a″″ to the atmosphere. The exhaustion of gas is shown schematically in FIG. 54 by the dashed arrow that begins at side port 164″″ and ends at outlet aperture 169a″″. During this exhaust process, activated carbon filter 166″″ filters the gas to capture toxic constituents and prevent them from escaping to the atmosphere. Activated carbon filter 166″″ works in series with hydrophobic filter 162″″ to filter gas that leaves the second fluid reservoir before being discharged to the atmosphere.

Flow passage 163″″ also provides a ventilation route for vent line 160″″ which allows air from the atmosphere to enter through outlet aperture 169a″″, pass through activated carbon filter 166″″ and into side port 164″″. This can assist in equalizing pressure in the CSTD to facilitate transfer of fluid. The ventilation of air is shown schematically in FIG. 54 by the solid arrow connecting outlet aperture 169a″″ to side port 164″″.

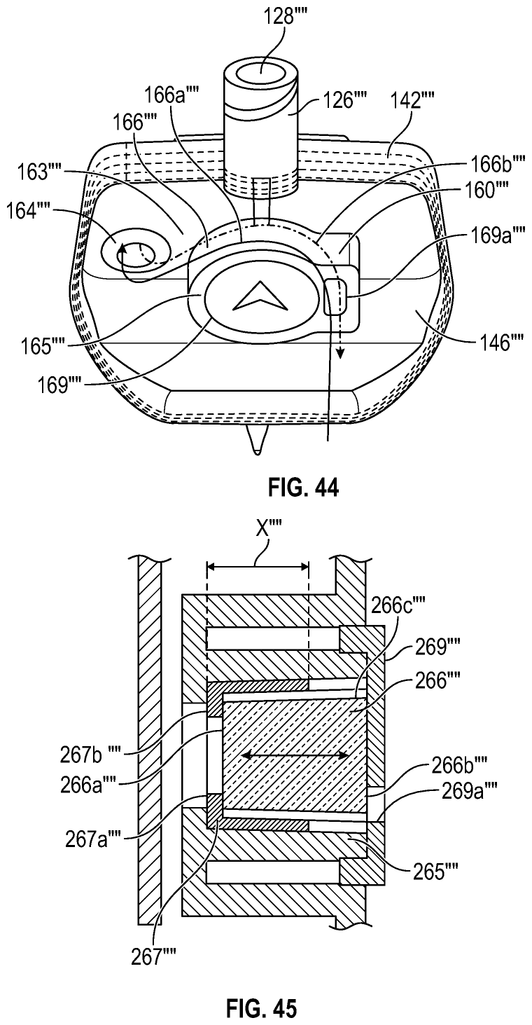

Activated carbon filters according to the present disclosure can have various geometries. In the present example, activated carbon filter 166″″ is a cylindrical-shaped filter medium having an interior face 166a″″, exterior face 166b″″ and circumferential sidewall 166c″″. Gas flows through activated carbon filter 166″″ in a “lateral” direction, as shown in FIG. 44. That is, the gas does not pass through the interior face 166a″″ or exterior face 166b″″. Instead, gas passes through sidewall 166c″″″. This can provide a longer flow path and ensure that the entire volume of the filter is utilized to filter hazardous components, allowing more filtering to take place and increasing filter capacity.

FIG. 45 shows an alternative arrangement, in which an activated carbon filter 266″″ filters gas flowing in an “axial” direction shown by the double ended arrow. A filter housing 265″″ contains activated carbon filter 266″″, which in this embodiment has a frustoconical shape, with an axial length longer than the axial length of activated carbon filter 166″″ Activated carbon filter 266″″ has an interior face 266a″″, exterior face 266b″″ and sidewall 266c″″. A carrier element 267″″ formed of a gas impermeable material, such as silicone, surrounds a portion of the interior face 266a″″ and sidewall 266c″″. Carrier element 267″″ has an opening 267a″″ that exposes a portion of interior face 266a″″ to allow gas to enter and exit that portion of the interior face. Exterior face 266b″″ of activated carbon filter 266″″ is covered by a cap 269″″ having a small outlet aperture 269a″″. Carrier element 267″″ has a side wall 267b″″ that extends a distance X corresponding to a portion of the axial length of activated carbon filter 266″″. In this arrangement, carrier element 267″″ and cap 269″″ allow gas to enter and exit portions of the interior face 266a″″ and exterior face 266b″″ and flow through activated carbon filter in an axial direction. Carrier element 267″ is compressed into a space between sidewall 266c″″ of activated carbon filter 266″″ and filter housing 265″″, forming a fluid-tight seal that prevents gas from flowing in a lateral direction through the activated carbon filter in the area covered. Gas entering activated carbon filter 266″″ through interior face 266a″″ is guided in an axial direction for at least the length X through the filter.

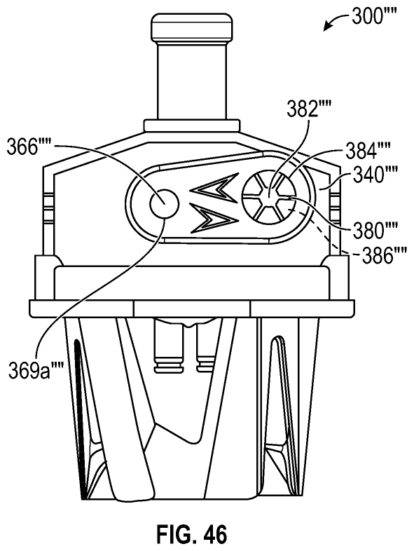

CSTDs according to the present disclosure can optionally include one or more mechanisms for allow air to enter the CSTD in a regulated manner. For example, CSTDs according to the present disclosure can have a variety of check valve configurations. FIG. 46 shows a CSTD 300″″ according to an alternate embodiment which features a housing 340″″ containing an activated carbon filter 366″″ and having an outlet aperture 369a″″. A check valve 380″″ is positioned next to outlet aperture 369a″″. Check valve 380″″ has a valve element 382″″ and a valve opening 384″″. Valve element 382″″ is a disc or other plate-like element configured to allow air to flow through valve opening 384″″ in one direction only. Valve element 382″″ is operable in an open position to allow air to enter valve opening 382″″ and into CSTD 300″″, and a closed position to prevent air from entering the valve opening and CSTD. A biasing element 386″″, which may be a compression spring, leaf spring or other spring element, holds valve element 382″″ in the closed position when the biasing element is in a relaxed or relatively relaxed state. Biasing element 386″″ is configured to compress, bend or otherwise deflect when atmospheric pressure outside valve opening 384″″ exceeds pressure inside housing 340″″ by a certain threshold. When the threshold is exceeded, the biasing element is deflected to allow valve element 382″″ to move to the open position and admit air through valve opening 384″″ and into housing 340″″.

Referring back to FIG. 38, vial clip 190″″ has a proximal end 192″″ with a fastener mechanism 193″″. Fastener mechanism 193″″ is configured to connect vial clip 190″″ to a vial spike, such as vial spike 120″″. Vial clips according to the present disclosure can feature a variety of fastener mechanisms for detachably connecting the vial clips to vial spikes, including one or more snap fit connectors. In the present embodiment, fastener mechanism 193″″ is made up of four flexible arms 196″″. Each flexible arm 196″″ is bendable between a relaxed state and a flexed state. In the relaxed state, each flexible arm 196″″ extends parallel to longitudinal axis Y. In the flexed state, each flexible arm 196″″ is bent radially outwardly and away from longitudinal axis Y with energy stored in the arm. Each flexible arm 196″″ has a barbed end 197″″ with an inwardly sloped face 198″″. Some of the barbed ends 197″″ and inwardly sloped faces 198″″ are not labeled for clarity.

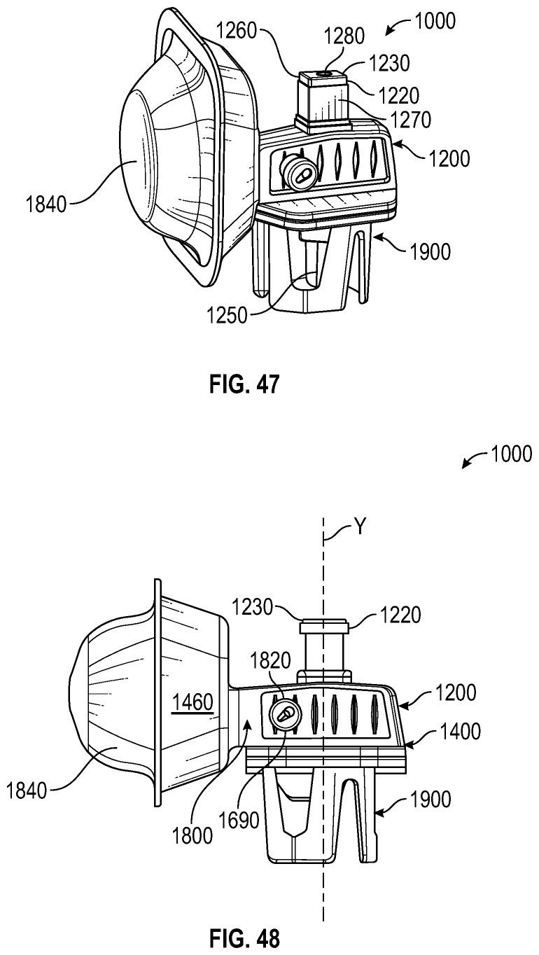

The flexible arms 196″″ are spaced apart such that the barbed ends 197″″ and inwardly sloped faces 198″″ abut second cover piece 145″″ of vial spike 120″″ as the vial spike is connected to vial clip 190″″. As second cover piece 145″″ comes into contact with inwardly sloped faces 198″″, the flexible arms 196″″ bend radially outwardly under stored energy to allow the second cover piece to pass between barbed ends 197″″. Once the second cover piece 145″″ and first cover piece 143″″ clear barbed ends 197″″, the stored energy in flexible arms 196″″ is released, and the flexible arms snap back to their relaxed state. At this point, the barbed ends 197′″ are positioned over first cover piece 143″″ and bear against the first cover piece to secure vial clip 190″″ onto vial spike 120″″, as shown in FIG. 47.

Vial clip 190″″ includes downwardly descending arcuate flanges 193″″ that form a generally cylindrical receptacle 199″″. In the assembled state, receptacle 199″″ receives spike connector 130″″ with flanges 193″″ surrounding the spike connector. In this arrangement, flanges 193″″ form a guard 195″″ that protects users from accidentally being sticked by pointed tip 134″″. Receptacle 199″″ is also configured to receive and surround a portion of a second fluid reservoir (e.g. the neck of a vial) to connect vial spike 120″″ to the second fluid reservoir.

Referring to FIGS. 47-50, a CSTD 1000 is shown according to another embodiment. CSTD 1000 includes an assembly referred to as a “vial spike” 1200 connected to a base or “vial clip” 1900. CSTD 1000 has a longitudinal axis Y which passes through center portions of vial spike 1200 and vial clip 1900. Vial spike 1200 has a proximal end 1220 forming a first connector 1230 configured to fluidly connect to a first fluid reservoir, such as a syringe. Vial spike 1200 also has a distal end 1240 forming a second connector 1250 configured to fluidly connect to a second fluid reservoir, such as a vial. Once vial spike 1200 is connected in fluid communication with first and second fluid reservoirs, respectively, the vial spike forms a closed fluid passage between the first and second fluid reservoirs. This closed fluid passage, which is also referred to herein as a “transfer line”, allows liquid to be transferred between the first and second fluid reservoirs in a sealed manner that prevents release of hazardous drugs into the environment.

First connector 1230 is a dry break coupling 1260. Dry break coupling 1260 has a rectangular body 1270 defining a fluid passage 1270a. A septum 1280 is mounted in fluid passage 1270a inside rectangular body 1270. Rectangular body 1270 is configured to mate with and fluidly connect to a syringe or other fluid reservoir.

Second connector 1250 is a spike connector 1300 having a cylindrical profile 1320 and pointed tip 1340. Spike connector 1300 has separate fluid passages that convey liquid and gas through the CSTD, as will be explained.

Vial spike 1200 has a three-part housing 1400 that forms passageways for liquid and gas flowing within CSTD 1000. Housing 1400 includes a first housing portion 1420 adjacent to and fluidly connected with dry break coupling 1260. Housing 1400 also includes a second housing portion 1440 adjacent to and fluidly connected with spike connector 1300. Moreover, housing 1400 includes a third housing portion 1460 which attaches to one end of first housing portion 1420.

First housing portion 1420 has a first cover piece 1430 opposite dry break coupling 1260. Second housing portion 1440 has second cover piece 1450 opposite spike connector 1300. Second cover piece 1450 is configured to connect with first cover piece 1430 to join the first housing portion 1420 and second housing portion 1440 together. First cover piece 1430 and second cover piece 1450 are shaped so as to as to form a narrow gap, void, or space 1410 between first housing portion 1420 and second housing portion 1440. A ring-shaped lip portion 1470 extends around the periphery of first cover piece 1430 and projects from first housing portion 1420. A ring-shaped wall portion 1480 extends around the periphery of second cover piece 1450, forming a receptacle slightly larger in size than the ring-shaped lip portion 1470. Ring-shaped wall portion 1480 is adapted to receive ring-shaped lip portion 1470 to join first housing portion 1420 to second housing portion 1440 in a mated and sealed arrangement.

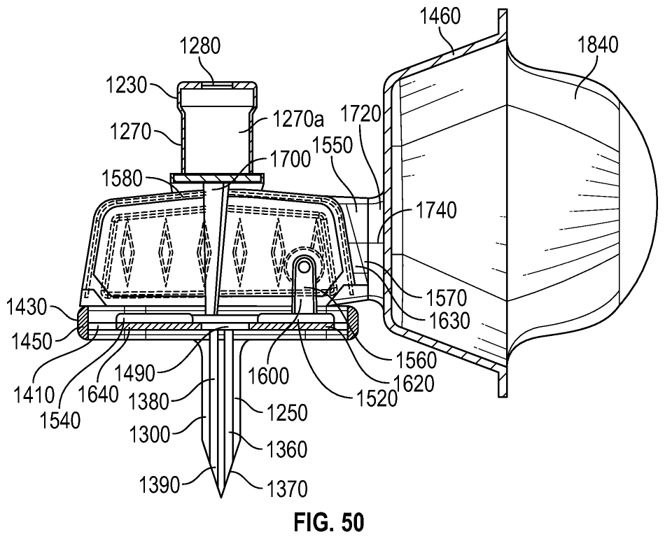

First cover piece 1430 forms a divider wall 1490, shown in FIG. 50, that spans the center of ring-shaped lip portion 1470. When first cover piece 1430 is mated with second cover piece 1450, ring-shaped wall portion 1470 and divider wall 1490 create two separate chambers inside the first and second cover pieces. A first chamber 1520 is formed on a first side of divider wall 1490, and a second chamber 1540 is formed on a second side of the divider wall opposite the first side.

First cover piece 1430 and second cover piece 1450 are connected to one another so that they fully enclose first chamber 1520 and second chamber 1540 and seal the chambers in a fluid tight manner from the exterior of vial spike 1200. In addition, first cover piece 1430 and second cover piece 1450 are connected to one another to seal first chamber 1520 in a fluid tight manner from second chamber 1540, and vice versa. In this regard, first cover piece 1430 can be joined to second cover piece 1450 using any suitable means that seals the chambers from the exterior of vial spike 1200, and seals the chambers from one another. Suitable means for establishing such a sealed arrangement include welding techniques such as ultrasonic welding, hot plate welding and laser welding. Suitable means for establishing a sealed arrangement also include over molding and gluing.

Spike connector 1300 has a first passage 1360 and a second passage 1380 extending parallel to the first passage. When vial spike 1200 is fully assembled, first passage 1360 fluidly connects with first chamber 1520 but not with second chamber 1540. In addition, second passage 1380 fluidly connects to second chamber 1540 but not first chamber 1520. First passage 1360 extends the entire length of spike connector 1300 and exits pointed tip 1340 where it forms a first opening 1370. Second fluid passage 1380 also extends the entire length of spike connector 1300 and exits pointed tip 1340 where it forms a second opening 1390. First chamber 1520 fluidly connects to a vent passage 1560 in first housing portion 1420. Second chamber 1540 connects to a fluid conduit 1580 which in turn, fluidly connects to fluid passage 1270a of dry break coupling 1260. In this arrangement, first passage 1360 forms part of a vent line 1600 that equalizes pressure in CSTD 1000, as will be explained, while second passage 1380 forms part of a transfer line 1700 for transferring liquid between the first fluid reservoir and second fluid reservoir. Vent line 1600 and transfer line 1700 are sealed from one another within CSTD 1000, so that gas carried in vent line 1600 does not enter transfer line 1700, and liquid carried in the transfer line does not enter the vent line.

Each chamber in CSTD 1000 is configured to contain a filter material or filter component dedicated to each respective line. The chambers need not contain any filters, however. In the present example, first chamber 1520 contains a first filter component in the form of a hydrophobic filter 1620, and second chamber 1540 contains a second filter component in the form of a particle filter 1640 arranged in parallel with the hydrophobic filter in a coplanar arrangement.

CSTD 1000 has a “pressure balancing” mechanism 1800 designed to equalize pressure when a pressure gradient occurs between the vessels, or between the interior and exterior of the device. A pressure gradient can occur when CSTD 1000 is attached to a vial and a syringe, and liquid is transferred between the vial and syringe through the device, such as during a withdrawal of liquid from the vial or injection of liquid into the vial.

Pressure balancing mechanism 1800 includes a check valve 1820 connected to one side of first housing portion 1420 and an inflatable barrier or membrane 1840 connected to third housing portion 1460. Membrane 1840 is configured to expand in response to gas flowing into third housing portion 1460, and configured to collapse in response to the discharge of gas out of the third housing portion. When gas is released from a vial after being penetrated with spike connector 1300, the gas will travel through the spike connector, second housing portion 1440, first housing portion 1420 and into third housing portion 1460. Once inside third housing portion 1460, the gas is trapped and collected by membrane 1840. Membrane 1840 is configured to expand when third housing portion 1460 receives gas to balance pressure in CSTD 1000. This gas can remain stored in third housing portion 1460 and membrane 1840 until a pressure drop occurs in CSTD 1000. When a pressure drop suddenly occurs in CSTD 1000, gas stored in third housing portion 1460 and membrane 1840 is released into first housing portion 1420 to balance pressure. If additional gas is needed to balance pressure, check valve 1820 is configured to allow air outside CSTD 1000 to flow into first housing portion 1420 to balance the pressure, as will be described.

Vent line 1600 allows gas from third housing portion 1460 and membrane 1840 to flow into other parts of CSTD 1000 to balance pressure. In addition, vent line 1600 allows air entering CSTD 1000 through check valve 1820 to flow into different parts of CSTD 1000 to balance pressure. Hydrophobic filter 1620 is configured to allow gases to flow to and from spike connector 1300 through the hydrophobic filter, while not allowing liquids and aerosols in the gases to pass through the hydrophobic filter. This prevents contaminants from the atmosphere from passing through the first chamber 1520, through the first passage 1360 and into the second fluid reservoir. It also prevents or substantially prevents liquids and aerosols from the second fluid reservoir from going beyond the first chamber 1520. Hydrophobic filter 1620 can have any suitable pore size, including but not limited to a pore size of 0.2 microns.

Particle filter 1640 is configured to allow liquid to pass through the particle filter, but not allow small particles from passing through the particle filter. This prevents particles from the first fluid reservoir from transferring to the second fluid reservoir, and vice versa. Particle filters according to the present disclosure can be formed of any suitable material for filtering particulates, including but not limited to membranes formed of acrylic copolymer, polyether sulfone or polyvinylidene fluoride.

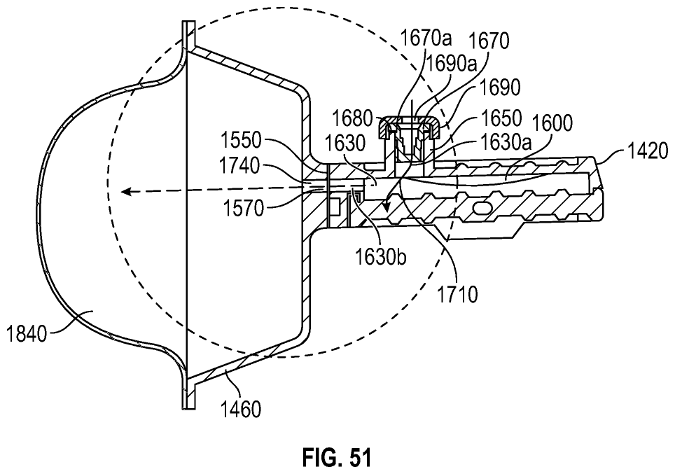

Referring to FIGS. 50 and 51, vent line 1600 extends from first passage 1360, through first chamber 1520 and into vent passage 1560 of first housing portion 1420. The interior of first housing portion 1420 provides an elongated flow passage 1630 that forms a section of vent line 1600. First housing portion 1420 defines a check valve housing 1650 on one side of flow passage 1630. Check valve housing 1650 contains a check valve 1670. First housing portion 1420 further includes an inlet 1680 adjacent to check valve 1670. Inlet 1680 is covered by a cap 1690 to retain check valve 1670 inside check valve housing 1650. Cap 1690 has a small aperture 1690a that connects with the atmosphere.

CSTDs according to the present disclosure can have a variety of check valve configurations. In the present example, check valve 1670 includes a resilient flexible valve element 1670a inside check valve housing 1650. Valve element 1670a is a cup-shaped element configured to allow air to flow through check valve housing 1650 in one direction only. In particular, valve element 1670a is movable between an open position to allow air to enter inlet 1680 and into first housing portion 1420, and a closed position to prevent air from leaving the first housing portion through the inlet. Valve element 1670a assumes the closed position when the valve element is in a relaxed or relatively relaxed state. In the closed position, valve element 1670a is pressed against inlet 1680 to prevent gas from entering or leaving first housing portion 1420. When atmospheric pressure outside inlet 1680 exceeds pressure inside first housing portion 1420 by a certain threshold, valve element 1670a moves inwardly and away from inlet 1680. This allows air to enter inlet 1680 and flow into first housing portion 1420 to balance pressure in CSTD 1000.

First housing portion 1420 has a first end 1550 configured to connect with a second end 1720 on third housing portion 1460. First end 1550 has an end opening 1570 at one end of first passage 1630. End opening 1570 is positioned to align with a mouth opening 1740 at second end 1720 when third housing portion 1460 is connected to first housing portion 1420. In this arrangement, end opening 1570 and mouth opening 1740 form a fluid passage that interconnects first housing portion 1420 with third housing portion 1460 in fluid communication. Second end 1720 can be connected to first end 1550 in a number of suitable ways, including welding techniques such as ultrasonic welding, hot plate welding and laser welding. Other suitable ways include over molding and gluing.

Check valve housing 1650 fluidly connects to first passage 1630 at a junction 1710. First passage 1630 extends through first housing portion 1420 and splits or branches in two different directions at junction 1710. That is, first passage 1630 splits into a first branch 1630a toward check valve 1670 and a second branch 1630b toward socket 1550.

Gas under positive pressure in second fluid reservoir 1640 exhausts out of CSTD 1000 by flowing up vent line 1600 and into flow passage 1630. Once the gas reaches junction 1710, the gas is prevented from exiting CSTD 1000 through first branch 1630a because valve element 1670a of check valve 1670 is moved to the closed position, blocking inlet 1680. Therefore, the gas at junction 1710 proceeds through second branch 1630b and end opening 1570, until it enters third housing portion 1460, as noted earlier. Membrane 1840 is configured to expand in response to gas entering into third housing portion 1460. The volume or size of membrane 1840 changes in response to gas entering the membrane. The increased size of membrane 1840 is visibly detectable from the outside of CSTD 1000, notifying the user that gas is being stored in third housing portion 1460, as opposed to exiting the CSTD. Exhaustion of gas from second fluid reservoir 1640 into membrane 1840 is shown schematically in FIG. 51 by the broken line arrow.

When a negative pressure gradient develops, gas stored under pressure in membrane 1840 can balance pressure. Gas exits third housing portion 1460 through mouth opening 1740 and enters flow passage 1630 in first housing portion 1620. From there, the gas can travel along vent line 1700 and enter second housing portion 1440 to balance pressure. If a sufficient amount of gas is not stored in membrane 1840 to balance pressure in CSTD 1000, and a pressure gradient remains, then higher pressure air from the outside atmosphere will open valve element 1670a and enter first housing portion 1620 through inlet 1680. Air will enter inlet 1680 and fill CSTD 1000 until a pressure equilibrium is reached between the interior of CSTD and the outside air. This can assist in equalizing pressure in the CSTD to facilitate transfer of fluid. The ventilation of air through inlet 1680 is shown schematically in FIG. 51 by the solid arrow.

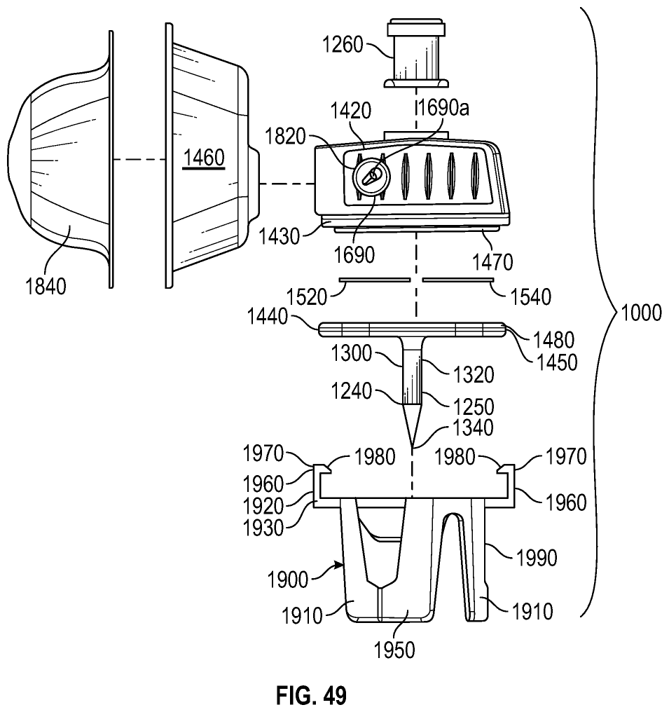

Referring back to FIGS. 48 and 49, vial clip 1900 has a proximal end 1920 with a fastener mechanism 1930. Fastener mechanism 1930 is configured to connect vial clip 1900 to a vial spike, such as vial spike 1200. Vial clips according to the present disclosure can feature a variety of fastener mechanisms for detachably connecting the vial clips to vial spikes, including one or more snap fit connectors. In the present embodiment, fastener mechanism 1930 is in the form of flexible arms 1960. Each flexible arm 1960 is bendable between a relaxed state and a flexed state. In the relaxed state, each flexible arm 1960 extends parallel or generally parallel to longitudinal axis Y. In the flexed state, each flexible arm 1960 is bent radially outwardly and away from longitudinal axis Y with energy stored in the arm. Each flexible arm 1960 has a barbed end 1970 with an inwardly sloped face 1980.

The flexible arms 1960 are configured such that the barbed ends 1970 and inwardly sloped faces 1980 abut second cover piece 1450 of vial spike 1200 as the vial spike is connected to vial clip 1900. As second cover piece 1450 comes into contact with inwardly sloped faces 1980, the flexible arms 1960 bend radially outwardly under stored energy to allow the second cover piece to pass between barbed ends 1970. Once second cover piece 1450 and first cover piece 1430 clear barbed ends 1970, the stored energy in flexible arms 1960 is released, and the flexible arms snap back to their relaxed state, acting like snap hooks. At this point, the barbed ends 1970 engage small ledges on opposite sides of first housing portion 1420 to connect vial clip 1900 to vial spike 1200.

Vial clip 1900 includes downwardly descending arcuate flanges 1910 that form a generally cylindrical receptacle 1990. In the assembled state, receptacle 1990 receives spike connector 1300 with flanges 1910 surrounding the spike connector. In this arrangement, flanges 1910 form a guard 1950 that protects users from accidentally being sticked by pointed tip 1340. Receptacle 1990 is also configured to receive and surround a portion of a second fluid reservoir (e.g. the neck of a vial) to connect vial spike 1200 to the second fluid reservoir.

Some CSTD's according to the present disclosure have components specifically designed for filters, or have components specifically designed for inflatable barriers. Examples of these CSTDs are shown in FIGS. 37-51. Other CSTD's according to the present disclosure have versatile components that can be assembled to either filters or inflatable barriers. This latter type of CSTD can be provided in a modular system that allows different sets of components to be selected and combined.

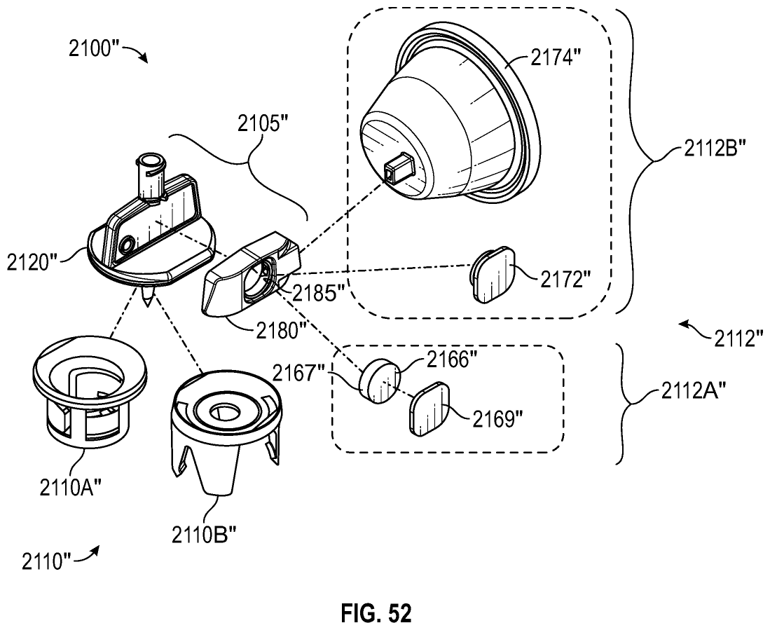

Referring to FIG. 52, an example of a modular system 2100″ featuring a vial adaptor is shown. Modular system 2100″ includes a set of components from which individual parts can be selected to assemble a CSTD having a desired application or function. Components can be selected and assembled to manufacture a CSTD that works with a specific type or size of fluid container or reservoir. In addition, components can be selected and assembled to manufacture a CSTD that equalizes pressure gradients in a certain way. For example, one subset of components, referred to herein as a “module”, can be selected to filter hazardous gas and discharge the filtered gas to the atmosphere. Another subset of components or module can be selected to capture hazardous gas and store the gas inside the CSTD. Different CSTDs can be customized and manufactured from a common set of components, increasing manufacturing efficiency.

Modular system 2100″ includes a core or “base assembly” 2105″ and four separate modules 2110A″, 2110B″, 2112A″ and 2112B″. Base assembly 2105″ forms a central construct around which different CSTDs can be assembled. Modules 2110A″ and 2110B″ are interchangeable clip modules 2110″ that allow base assembly 2105″ to connect to containers of different sizes depending on which clip module is chosen. Modules 2112A″ and 2112B″ are interchangeable venting modules 2112″ that allow pressure in a CSTD to be equalized in a certain way, depending on which venting module is chosen. Base assembly 2105″ is configured to connect to one clip module 2110″ at a time and one venting module 2112″ at a time to form a CSTD. Therefore, an assembled CSTD in this example can consist of base assembly 2105″ connected to one of the clip modules 2110″ and one of the venting modules 2112″.

Base assembly 2105″ is a subassembly of parts that can be assembled prior to adding a clip module 2110″ and venting module 2112″. The subassembly of parts includes a vial spike 2120″ and a gas exchange unit 2180″. Vial spike 2120″ can connect to either module 2110A″ or module 2110B″, and gas exchange unit 2180″ can connect to either module 2112A″ or module 2112B″. Vial spike 2120″ and gas exchange unit 2180″ are manufactured separately by injection molding and subsequently joined together using ultrasonic welding or other joining technique.

Base assembly 2105″ manages the flow of both liquids and gases through a CSTD after the CSTD is connected to first and second fluid reservoirs. Vial spike 2120″ forms a transfer passage that allows liquid in the first fluid reservoir to pass to the second fluid reservoir in a sealed environment. Gas exchange unit 2180″ forms part of a vent passage that equalizes pressure within the CSTD after vial spike 2120″ is connected to the first and second fluid reservoirs. The transfer passage and vent passage are physically separated from one another, as will be explained.

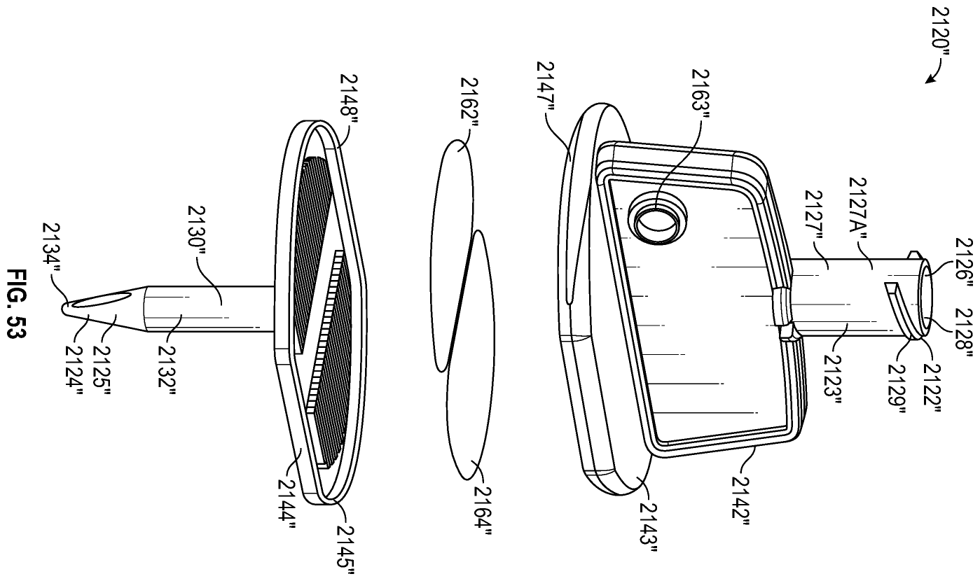

Referring to FIGS. 53 and 54, vial spike 2120″ has a proximal end 2122″ forming a first connector 2123″ configured to fluidly connect to a first fluid reservoir, such as a syringe. Vial spike 2120″ also has a distal end 2124″ forming a second connector 2125″ configured to fluidly connect to a second fluid reservoir, such as a vial. Once vial spike 2120″ is connected in fluid communication with first and second fluid reservoirs, respectively, the vial spike forms a closed fluid passage between the first and second fluid reservoirs. This closed fluid passage, which is also referred to herein as a “transfer line”, allows liquid to be transferred between the first and second fluid reservoirs in a sealed manner that prevents release of hazardous drugs into the environment.

Vial spikes according to the present disclosure can have a variety of fluid connectors at the proximal and distal ends. In the present embodiment, first connector 2123″ is a Luer lock connector 2126″. Luer lock connector 2126″ has a body 2127″ defining a fluid passage 2127A″. A septum 2128″ is mounted in fluid passage 2127A″ inside body 2127″. Body 2127″ has an external thread 2129″ configured to mate with an internal thread on syringe or other fluid reservoir.

Second connector 2125″ is a spike connector 2130″ having a cylindrical profile 2132″ and pointed tip 2134″. Spike connector 2130″ has separate fluid passages that convey liquid and gas through the CSTD as will be explained.

Vial spike 2120″ has a two-part housing 2140″ that forms passageways for liquid and gas flowing within a CSTD. Housing 2140″ includes a first housing portion 2142″ adjacent to and fluidly connected with Luer lock connector 2126″. Housing 2140″ also includes a second housing portion 2144″ adjacent to and fluidly connected with spike connector 2130″.

First housing portion 2142″ has a first cover piece 2143″ opposite Luer lock connector 2126″. Second housing portion 2144″ has second cover piece 2145″ opposite spike connector 2130″. Second cover piece 2145″ is configured to connect with first cover piece 2143″ to join the first housing portion 2142″ and second housing portion 2144″ together. First cover piece 2143″ and second cover piece 2145″ form an enlarged flange section that functions as a finger stop or finger rest, providing more comfort when the device is held.

First cover piece 2143″ and second cover piece 2145″ also form a narrow gap, void, or space 2141″ between first housing portion 2142″ and second housing portion 2144″. A ring-shaped lip portion 2147″ extends around the periphery of first cover piece 2143″ and projects from first housing portion 2142″. A ring-shaped wall portion 2148″ extends around the periphery of second cover piece 2145″, forming a receptacle slightly larger in size than the ring-shaped lip portion 2147″. Ring-shaped wall portion 2148″ is adapted to receive ring-shaped lip portion 2147″ to join first housing portion 2142″ to second housing portion 2144″ in a mated and sealed arrangement.

Referring to FIGS. 54 and 55, first cover piece 2143″ has a divider wall 2149″ that spans the center of ring-shaped lip portion 2147″ as shown. When first cover piece 2143″ is mated with second cover piece 2145″, ring-shaped wall portion 2148″ and divider wall 2149″ create two separate chambers inside the first and second cover pieces. A first chamber 2152″ is formed on a first side of divider wall 2149″, and a second chamber 2154″ is formed on a second side of the divider wall opposite the first side.

First cover piece 2143″ and second cover piece 2145″ are connected to one another so that they fully enclose first chamber 2152″ and second chamber 2154″ and seal the chambers in a fluid tight manner from the exterior of vial spike 2120″. In addition, first cover piece 2143″ and second cover piece 2145″ are connected to one another to seal first chamber 2152″ in a fluid tight manner from second chamber 2154″, and vice versa. In this regard, first cover piece 2143″ can be joined to second cover piece 2145″ using any suitable means that seals the chambers from the exterior of vial spike 2120″, and seals the chambers from one another. Suitable means for establishing such a sealed arrangement include welding techniques such as ultrasonic welding, hot plate welding and laser welding, or other joining techniques. Suitable means for establishing a sealed arrangement also include over molding and gluing.

Spike connector 2130″ has a first passage 2136″ and a second passage 2138″ extending parallel to the first passage. When vial spike 2120″ is fully assembled, first passage 2136″ fluidly connects with first chamber 2152″ but not with second chamber 2154″. In addition, second passage 2138″ fluidly connects to second chamber 2154″ but not first chamber 2152″. First passage 2136″ extends the entire length of spike connector 2130″ and exits pointed tip 2134″ where it forms a first opening 2137″. Second fluid passage 2138″ also extends the entire length of spike connector 2130″ and exits pointed tip 2134″ where it forms a second opening 2139″. First chamber 2152″ fluidly connects to a vent passage 2156″ in first housing portion 2142″. Second chamber 2154″ fluidly connects to fluid passage 2127A″ of Luer lock connector 2126″. In this arrangement, first passage 2136″ forms part of a vent line 2160″ that equalizes pressure in modular system 2100″ with outside atmospheric pressure, as will be explained, while second passage 2138″ forms part of a transfer line 2170″ for transferring liquid between the first fluid reservoir and second fluid reservoir. Vent line 2160″ and transfer line 2170″ are sealed from one another within the CSTD, so that gas carried in vent line 2160″ does not enter transfer line 2170″, and liquid carried in the transfer line does not enter the vent line.

Each chamber in modular system 2100″ is configured to contain a filter material or filter component dedicated to each respective line. In the present example, first chamber 2152″ contains a first filter component in the form of a hydrophobic filter 2162″, and second chamber 2154″ contains a second filter component in the form of a particle filter 2164″ arranged in parallel with the hydrophobic filter in a coplanar arrangement. Particle filter 2164″ is optional, depending on the application.

The enlarged flange shape of first and second cover pieces 2143″ and 2145″ provide an enlarged cross sectional area inside the cover pieces. That is, the cross sectional area of first chamber 2152″ is much larger than the cross sectional area of first passage 2136″, and the cross sectional area of second chamber 2154″ is much larger than the cross sectional area of second passage 2138″. The large cross sectional areas of first and second chamber 2152″ and 2154″ allow larger filters to be used. This increases the filter surface area and increases flow rate through the chambers.

Vent line 2160″ allows air from the atmosphere to enter the CSTD, as will be explained. Hydrophobic filter 2162″ is configured to allow air from the atmosphere to pass through the hydrophobic filter, while not allowing liquids and aerosols in the air to pass through the hydrophobic filter. This prevents contaminants from the atmosphere from passing through the first chamber 2152″, through the first passage 2136″ and into the second fluid reservoir. It also prevents or substantially prevents liquids and aerosols from the second fluid reservoir from going beyond the first chamber 2152″. Hydrophobic filter 2162″ can have any suitable pore size, including but not limited to a pore size of 0.2 microns.

Particle filter 2164″ is configured to allow liquid to pass through the particle filter, but not allow small particles from passing through the particle filter. This prevents particles in the first fluid reservoir from transferring to the second fluid reservoir, and vice versa. Particle filters according to the present disclosure can be comprised of any suitable material for filtering particulates, including but not limited to membranes formed of acrylic copolymer, polyether sulfone or polyvinylidene fluoride.

Vent line 2160″ extends from first passage 2136″, through first chamber 2152″ and into first housing portion 2142″. Vent line 2160″ exits first housing portion 2142″ through a side port 163 and into gas exchange unit 2180″.

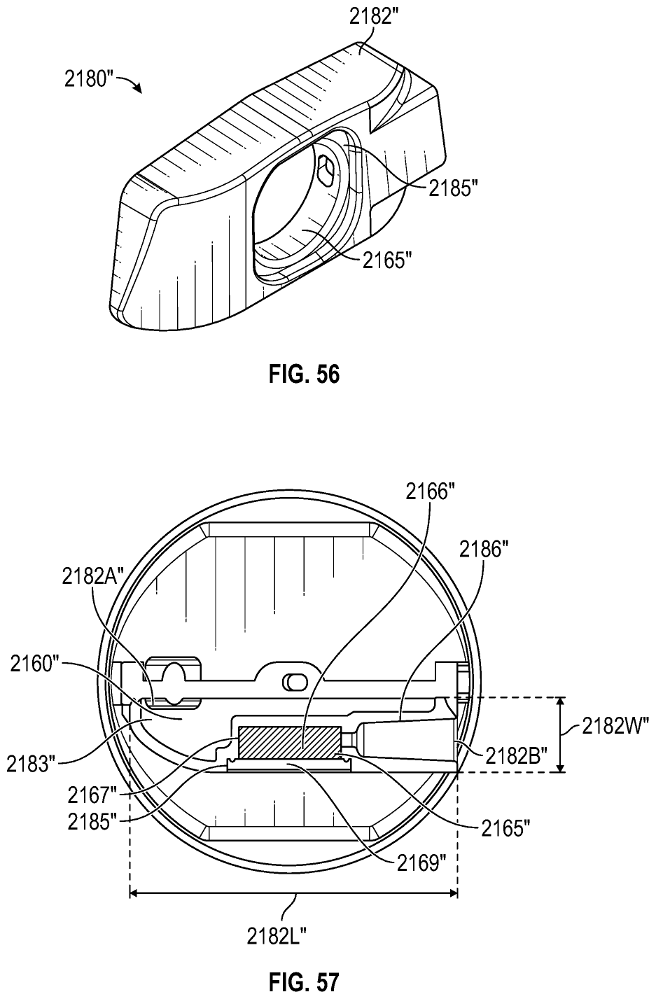

Referring to FIGS. 56 and 57, gas exchange unit 2180″ has a hollow shell structure or body 2182″ that connects to one side of first housing portion 2142″ and over side port 163. Body 2182″ has a body width 2182W″ and a body length 2182L″ that is significantly larger than the body width, creating a narrow profile. The interior of body 2182″ provides a gas exchange passage 2181″ that forms a section of vent line 2160″. Gas exchange passage 2181″ enters body 2182″ through a first port 2182A″ and fluidly connects with a plug-in receptacle 2185″ located in a center portion of the body. Gas exchange passage 2181″ exits plug-in receptacle 2185″ and fluidly connects to an elongated socket 2186″. Socket 2186″ exits body 2182″ through a second port 2182B″ to the exterior of the body.

Gas exchange unit 2180″ is configured to connect to and receive venting module 2112A″ or venting module 2112B″. Venting module 2112A″ converts gas exchange unit 2180″ to a filter unit that filters hazardous gas from the CSTD before discharging the gas to the atmosphere. Venting module 2112B″ converts gas exchange unit 2180″ to a gas storage unit that retains hazardous gas inside the CSTD and prevents the gas from being released to the atmosphere. In this arrangement, venting modules 2112A″ and 2112B″ provide interchangeable subassemblies that work with base assembly 2105″ to provide two different options for managing hazardous gas and ventilation in the CSTD. This allows different CSTDs to be manufactured and assembled using one base assembly design, increasing manufacturing efficiency as noted earlier.

Referring back to FIG. 52, venting module 2112A″ includes an activated carbon filter 2166″ designed to filter hazardous gas in vent line 2160″. Plug-in receptacle 2185″ forms a filter housing 2165″ adapted to receive activated carbon filter 2166″. Venting module 2112A″ also includes a cap 169 that closes plug-in receptacle 2185″ in an airtight arrangement after activated carbon filter 2166″ is received in filter housing 2165″ to retain and protect the activated carbon filter inside gas exchange unit 2180″. During operation, hazardous gas in vent line 2160″ passes into gas exchange passage 2181″ and through activated carbon filter 2166″ where it is filtered to remove toxins. The filtered gas then exits filter housing 2165″ and passes through elongated socket 2186″ to second port 2182B″ where the gas releases to the atmosphere.

Gas exchange passage 2181″ provides a two-way ventilation route for vent line 2160″ when venting module 2112A″ is used with gas exchange unit 2180″. That is, gas can flow through gas exchange passage 2181″ in a first direction, where the gas is filtered through activated carbon filter 2166″ and released to the atmosphere, as described above. In addition, gas exchange passage 2180″ allows air from the atmosphere to enter through second port 2182B″ and flow through vent line 2160″ in a second direction opposite the first direction to equalize pressure in the CSTD.

Activated carbon filters according to the present disclosure can have various geometries. In the present example, activated carbon filter 2166″ is a cylindrical-shaped filter medium. Gas flows through a sidewall 167 of activated carbon filter 2166″ in a “lateral” direction. This provides a longer flow path and ensures that the entire volume of the filter is utilized to filter hazardous components, allowing more filtering to take place and increasing filter capacity.

Venting module 2112B″ includes a check valve 2172″ in combination with an inflatable barrier or reservoir 2174″. Plug-in receptacle 2185″ is configured to connect to and receive check valve 2172″. Check valve 2172″ is a one-way valve that allows air from the atmosphere to enter gas exchange unit 2180″ but prevents gas from exiting the gas exchange unit. Check valve 2172″ is thus configured to open to allow air from the atmosphere to enter gas exchange unit 2180″ and flow into vent line 2160″ to equalize pressure in the CSTD. Check valve 2172″ is also configured to close in response to positive pressure in the gas exchange passage 2182″, such as when hazardous gas is released from a reservoir into the gas exchange passage, preventing the hazardous gas from escaping to the atmosphere through the check valve.

Reservoir 2174″ is configured to connect to second port 2182B″ to form an expandable storage balloon attached to gas exchange unit 2180″. In this arrangement, reservoir 2174″ is configured to collect gas that enters gas exchange unit 2180″ from vent line 2160″. Reservoir 2174″ is also configured to discharge stored gas into the gas exchange passage 2181″ and vent line 2160″ to balance pressure when pressure drops in the CSTD. When reservoir 2174″ collects gas, the membrane expands, providing a visible indicator or alert that hazardous gas is being released from one of the reservoirs and stored inside the CSTD. When reservoir 2174″ discharges gas, the membrane collapses, creating a visible indicator or alert that pressure is being equalized in the CSTD.

When gas is released from a vial after being penetrated with spike connector 2130″, the gas will travel through vial spike 2120″ and into gas exchange unit 2180″. Once inside gas exchange unit 2180″, the gas flows through plug-in receptacle 2185″ and is trapped and collected by reservoir 2174″. The trapped gas can remain stored in reservoir 2174″ until a pressure drop occurs in the CSTD. When a pressure drop occurs in the CSTD, gas stored in reservoir 2174″ is released into gas exchange passage 2181″ and vent line 2160″ to balance pressure. If additional gas is needed to balance pressure, check valve 2172″ is configured to allow air outside CSTD 2100″ to flow into first housing portion 2142″ to balance the pressure.

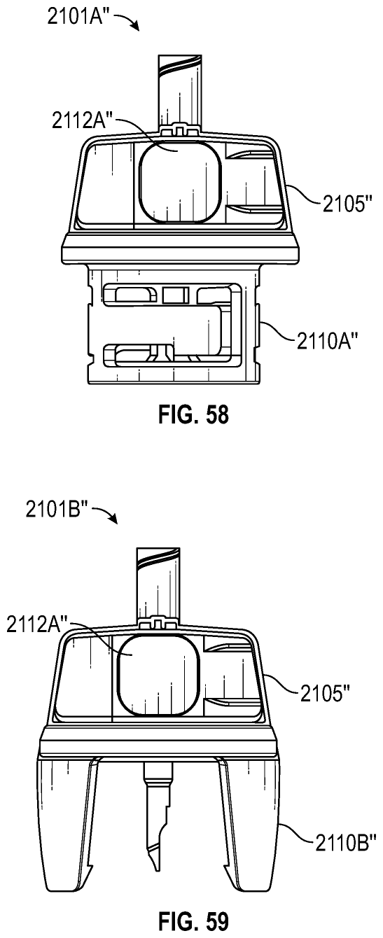

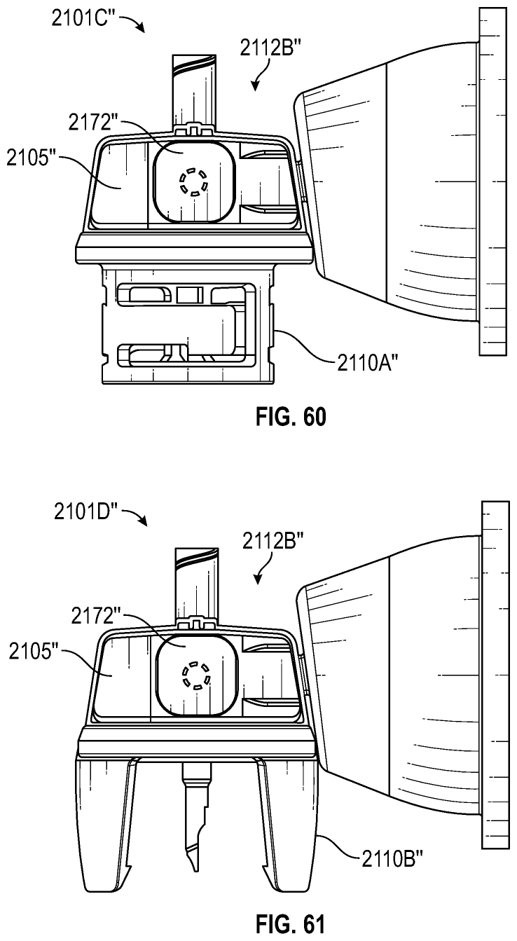

FIGS. 58-61 show four different CSTDs that can be assembled using modular system 2100″. In FIG. 58, CSTD 2101A″ includes base assembly 2105″ attached to clip module 2110A″ and venting module 2112A″. In FIG. 59, CSTD 2101B″ includes base assembly 2105″ attached to clip module 2110B″ and venting module 2112A″. In FIG. 60, CSTD 2101C″ includes base assembly 2105″ attached to clip module 2110A″ and venting module 2112B″. In FIG. 61, CSTD 2101D″ includes base assembly 2105″ attached to clip module 2110B″ and venting module 2112B″. Thus, the selection of each clip module can be done independently of which venting module is used, and vice versa.

Clip module 2110A″ is designed to connect to 13-20 mm vials. Clip module 2110B″ is designed to connect to 32 mm vials. It will be appreciated that clip modules according to the present disclosure can be configured to connect to any vial size or range of sizes, as well as other types of containers. Therefore, clip modules 2110A″ and 2110B″ should be understood to represent only two examples of modules that can be used with a base assembly according to the present disclosure.

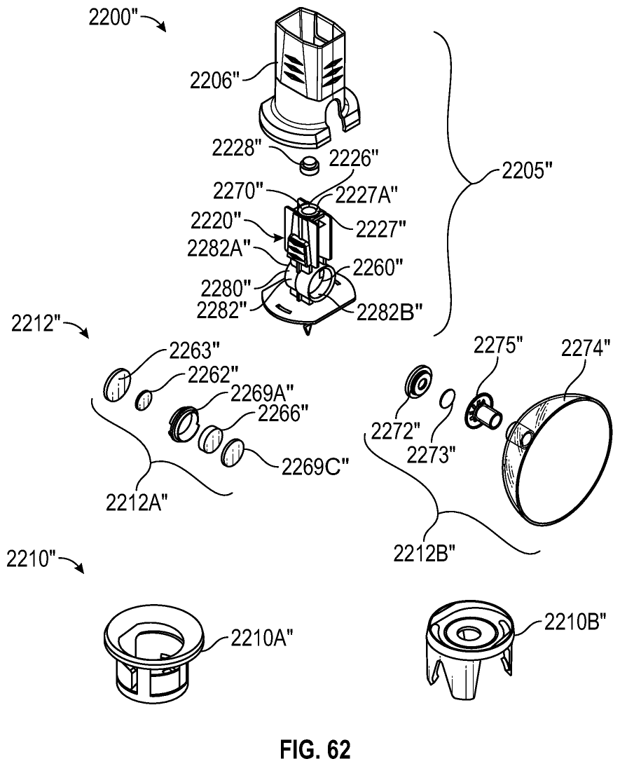

FIG. 62 shows a modular system 2200″ according to an alternative embodiment. Like modular system 2100″, modular system 2200″ includes a set of components from which individual parts can be selected to assemble a CSTD having a desired application or function. Components can be selected and assembled to manufacture a CSTD that works with a specific type or size of fluid container. In addition, components can be selected and assembled to manufacture a CSTD that equalizes pressure gradients in a certain way. For example, one subset of components can be selected to filter hazardous gas and discharge the filtered gas to the atmosphere. Another subset of components can be selected to capture hazardous gas and store the gas inside the CSTD. Different CSTDs can be customized and manufactured from a common set of components, increasing manufacturing efficiency.

Unlike modular system 2100″, modular system 2200″ features a core or base assembly 2205″ with a gas exchange unit 2280″ integrated into a vial spike 2220″. This arrangement reduces the number of parts that must be manufactured to form the base assembly 2205″, and it eliminates the step of joining the gas exchange unit 2280″ to the vial spike 2220″.

Modular system 2200″ also includes four separate modules 2210A″, 2210B″, 2212A″ and 2212B″ that can be individually selected for connection to base assembly 2205″, similar to modular system 2100″. Base assembly 2205″ forms a central construct out of which different CSTDs can be assembled via attachment to different modules. Modules 2210A″ and 2210B″ are interchangeable clip modules 2210″ that allow base assembly 2205″ to connect to containers of different sizes depending on which clip module is chosen. Modules 2212A″ and 2212B″ are interchangeable venting modules 2212″ that allow pressure in a CSTD to be equalized in a certain way, depending on which venting module is chosen.

Base assembly 2205″ is configured to connect with one clip module 2210″ and one venting module 2212″ to form a CSTD. Therefore, an assembled CSTD in this example can consist of base assembly 2205″, one clip module 2210″ and one venting module 2212″. Vial spike 2220″ can connect to either module 2210A″ or module 2210B″, and gas exchange unit 280 can connect to either module 2212A″ or module 212B. Base assembly 2205″ manages the flow of both liquids and gases through a CSTD after the CSTD is connected to first and second fluid reservoirs.

Base assemblies according to this embodiment can be manufactured with components and features that are the same as, or similar to, the components and features in modular system 2100″. Therefore, some of the components and features of base assembly 2205″ will not be described for brevity, with the understanding that those components and features can be the same components and features in base assembly 2105″ or substantially similar to those components and features.

Vial spike 2220″ forms a transfer line 2270″ that allows liquid in a first fluid reservoir to pass to a second fluid reservoir in a sealed environment. Gas exchange unit 2280″ forms part of a vent line 2260″ that equalizes pressure within the CSTD after vial spike 2220″ is connected to the first and second fluid reservoirs. The transfer line 2270″ and vent line 2260″ are physically separated from one another.

Base assembly 2205″ also includes an external housing 2206″ that is attachable over top of vial spike 2220″ and gas exchange unit 2280″. Vial spike 2220″ features a dry break coupling 2260″ defining a fluid passage 2227A″. Dry break coupling 2260″ prevents liquid or vapor from being released from the CSTD during coupling or decoupling of the vial spike from the first fluid reservoir. Dry break coupling 2260″ has a cylindrical body 2227″ defining a fluid passage 2227A″. Fluid passage 2227A″ forms part of the transfer line 2270″. A septum 2228″ is mounted in fluid passage 2227A″. Septum 2228″ acts in combination with a suitable coupling on the first fluid reservoir to form a sealed connection.

Gas exchange unit 2280″ has a cylindrical body 2282″ with a first open end 2282A″ and a second open end 2282B″. Referring to FIGS. 64 and 66, the interior of body 2282″ forms part of transfer line 2270″. The interior of body 2282″ also forms a gas exchange passage 2283″ which is sealed off from and fluidly separate from transfer line 2270″. Gas exchange passage 2283″, which forms a section of vent line 2260″, enters body 2282″ through a first port 2281″.

Referring back to FIG. 62, the first and second open ends 2282A″, 2282B″ are configured to connect with venting module 2212A″, or alternatively, with venting module 2212B″. The flow of gases through gas exchange passage 2283″ are altered and depend on which of the venting modules 2212″ is connected to gas exchange unit 2280″. Venting module 2212A″ converts gas exchange unit 2280″ to a filter unit that filters hazardous gas from the CSTD before discharging the gas to the atmosphere. Venting module 2212B″ converts gas exchange unit 2280″ to a gas storage unit that retains hazardous gas inside the CSTD and prevents the gas from being released to the atmosphere. In this arrangement, venting modules 2212A″ and 2212B″ provide interchangeable subassemblies that work with base assembly 2205″ to provide two different options for managing hazardous gas and ventilation in the CSTD.

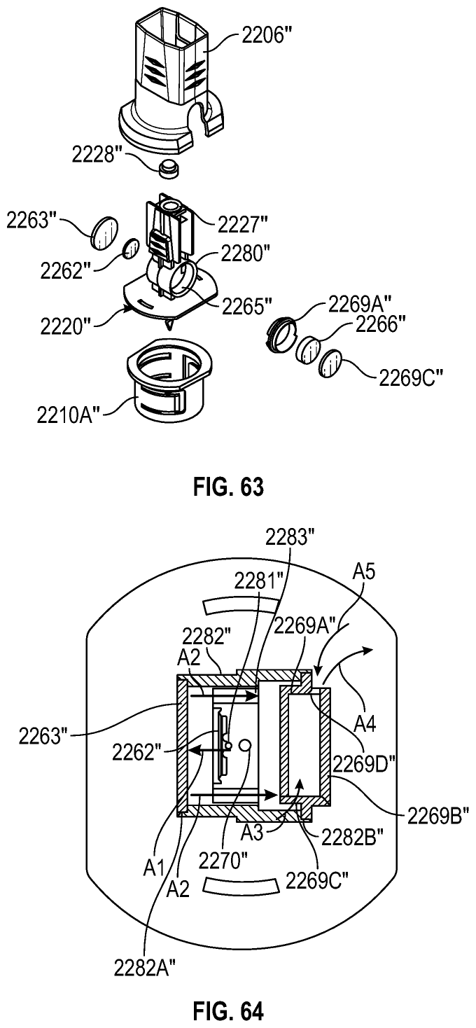

FIG. 63 shows components of venting module 2212A″, and FIG. 64 shows the interior of gas exchange unit 2280 with venting module 2212A″ installed. Venting module 2212A″ includes a hydrophobic air filter 2262″ that is positioned over first port 2281″ inside gas exchange unit 2280″. Gas that enters gas exchange passage 2283″ from a first reservoir is immediately filtered by hydrophobic air filter 2262″. The flow of gas through hydrophobic air filter 2262″ is shown by arrow A1. Venting module 2212A″ also includes a cover 2263″ that closes first open end 2282A″ in an air-tight arrangement to prevent the gas from exiting the first open end. Gas that enters gas exchange unit 2280″ passes through air filter 2262″ and flows inside gas exchange passage 2283″ toward second open end 2282B″, as represented by arrows A2.

Second open end 2282B″ forms a filter housing 2265″ that receives and filters toxins from the gas that passes through air filter 2262″. Venting module 2212A″ includes a filter case 2269A″, a filter cap 2269B″ and an activated carbon filter 2266″. Filter case 2269A″ is insertable into filter housing 2265″ in second open end 2282B″. Activated carbon filter 2266″ is a disk-shaped filter medium that can be inserted into filter case 2269A″ after the filter case is inserted into second open end 2282B″. Filter cap 2269B″ can be bonded to, snapped onto, or otherwise attached to filter case 2269A″ after activated carbon filter 2266″ is received in the filter case to retain and protect the activated carbon filter inside the gas exchange unit 2280″.

Filter case 2269A″ and filter cap 2269B″ form a first opening 2269C″ positioned toward the interior of gas exchange unit 2280″, and a second opening 2269D″ adjacent the exterior of the gas exchange unit. During operation, gas received from air filter 2262 enters filter case 2269A″ through first opening 2269C″, as shown by arrow A3. From there, the gas flows laterally through activated carbon filter 2266″ where it is filtered to remove toxins. The filtered gas then exits activated carbon filter 2266″ and discharges from filter case 2269A″ through second opening 2269D″ where the gas releases to the atmosphere, as shown by arrow A4.

Gas exchange passage 2283″ provides a two-way ventilation route for vent line 2260″ when venting module 2212A″ is used with gas exchange unit 2280″. That is, gas exchange passage 2283″ allows gas to be filtered through activated carbon filter 2266″ and released to the atmosphere, as noted above. In addition, gas exchange passage 2280″ allows air from the atmosphere to enter through second opening 2269D″, as represented by arrow A5, and flow into first port 2281″, whereafter the air passes into vent line 2260″ to equalize pressure in the CSTD so that fluid can be transferred between two reservoirs connected to the CSTD.

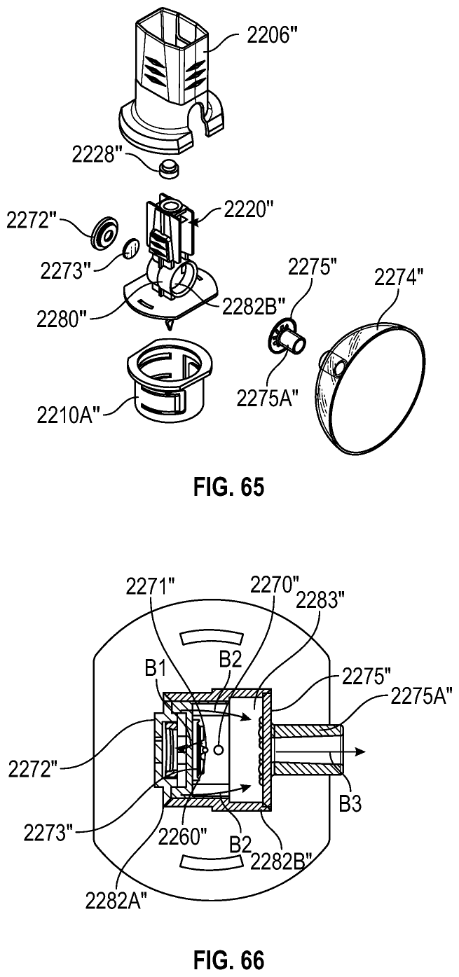

FIG. 65 shows components of venting module 2212B″, and FIG. 66 shows the interior of gas exchange unit 2280″ with venting module 2212B″ installed. Venting module 2212B″ includes a one-way check valve 2272″, a hydrophobic air filter 2273″, an inflatable reservoir, barrier or reservoir 2274″, and an adaptor 2275″. Adaptor 2275″ is configured to be inserted into second open end 2282B″. A tubular stem 2275A″ projects outwardly from second open end 2282B″ when adaptor 2275″ is connected to gas exchange unit 2280″. In this arrangement, reservoir 2274″ is attachable to stem 2275A″ to connect the membrane to gas exchange unit 2280″.

First open end 2282A″ is configured to receive hydrophobic air filter 2273″ and check valve 2272″ as shown. Hydrophobic air filter 2273″ connects over first port 2281″ (shown in FIG. 64), similar to hydrophobic air filter 2262″. In this respect, hydrophobic air filter 2273″ can be identical to hydrophobic air filter 2262″. Hazardous gas that enters gas exchange passage 2283″ from a first reservoir is immediately filtered by hydrophobic air filter 2273″. The flow of gas through hydrophobic air filter 2262″ is shown by arrow B1. Check valve 2272″ is a one-way valve that allows air from the atmosphere to enter gas exchange unit 2280″ but prevents gas from exiting the gas exchange unit. Therefore, the hazardous gas entering gas exchange passage 2283″ from first port is prevented from exiting first open end 2282A″, and is instead directed toward second open end 2282B″, as shown by arrows B2. From there, the hazardous gas passes through tubular stem 2275A″ as shown by arrow B3 and into barrier 2274″ where the gas is trapped in a sealed environment.

Check valve 2272″ is configured to open to allow air from the atmosphere to enter gas exchange unit 2280″ and flow into vent line 2260″ to equalize pressure in the CSTD. Check valve 2272″ is also configured to close in response to positive pressure in the gas exchange passage 2283″, such as when hazardous gas enters the gas exchange passage from first port 2281″. This prevents the hazardous gas from escaping to the atmosphere through the check valve.

Reservoir 2274″ is configured to connect to second open end 2282B″ to form an expandable storage balloon similar to reservoir 2174″. In this arrangement, reservoir 2274″ is configured to collect gas that enters gas exchange unit 2280″ from vent line 2260″. Reservoir 2274″ is also configured to discharge stored gas into the gas exchange passage 2283″ and vent line 2260″ when pressure drops in the CSTD to balance pressure. When reservoir 2274″ collects gas, the membrane expands, providing a visible indicator or alert that hazardous gas is being released from one of the reservoirs and stored inside the CSTD. When reservoir 2274″ discharges gas, the membrane collapses, creating a visible indicator or alert that pressure is being equalized in the CSTD. Thus, when gas is released from the vial after being penetrated with spike connector 2230″, for example, the gas will travel through vial spike 2220″ and into gas exchange unit 2280″. Once inside gas exchange unit 2280″, the gas flows through tubular stem 2275A″ and is trapped and collected by reservoir 2274″. The trapped gas can remain stored in reservoir 2274″ until a pressure drop occurs in the CSTD. When a pressure drop occurs in the CSTD, gas stored in reservoir 2274″ is released into gas exchange passage 2283″ and vent line 2260″ to balance pressure. If additional gas is needed to balance pressure, check valve 2272″ is configured to allow air outside the CSTD to flow into gas exchange unit 2280″ and vent line 2260″ to balance the pressure.

Clip modules 2210A″ and 2210B″ are equivalent to clip modules 2110A″ and 2110B″. Therefore, module 2210A″ is designed to connect to 13-20 mm vials, and clip module 2210B″ is designed to connect to 32 mm vials. The selection of each clip module can be done independently of which venting module is used, and vice versa.

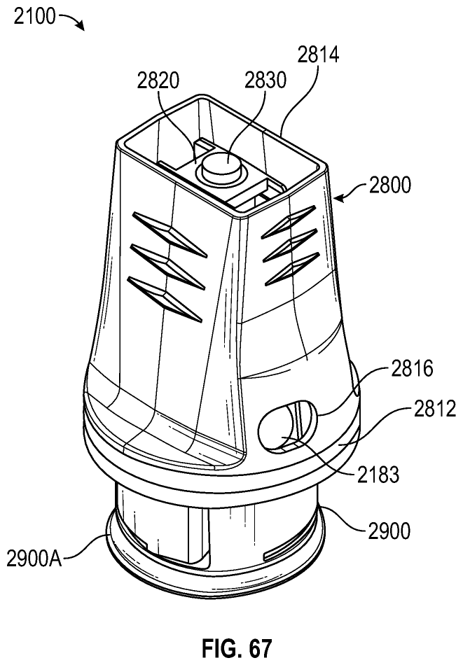

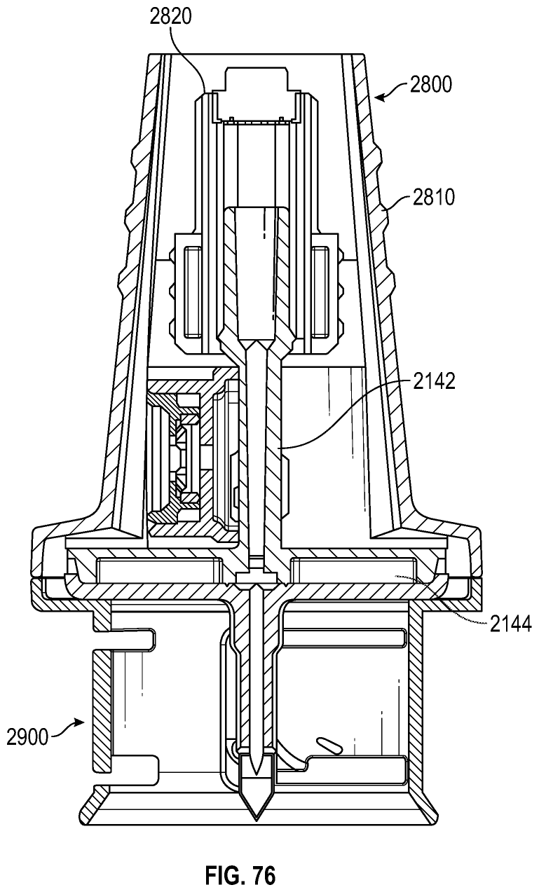

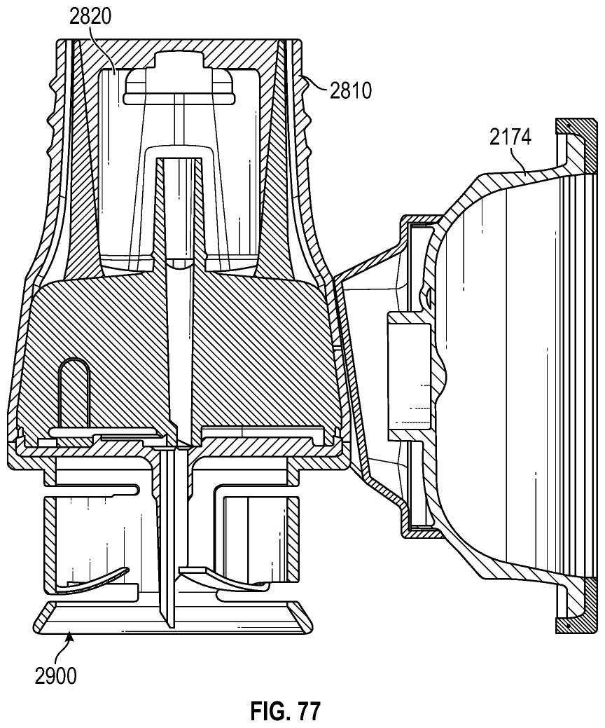

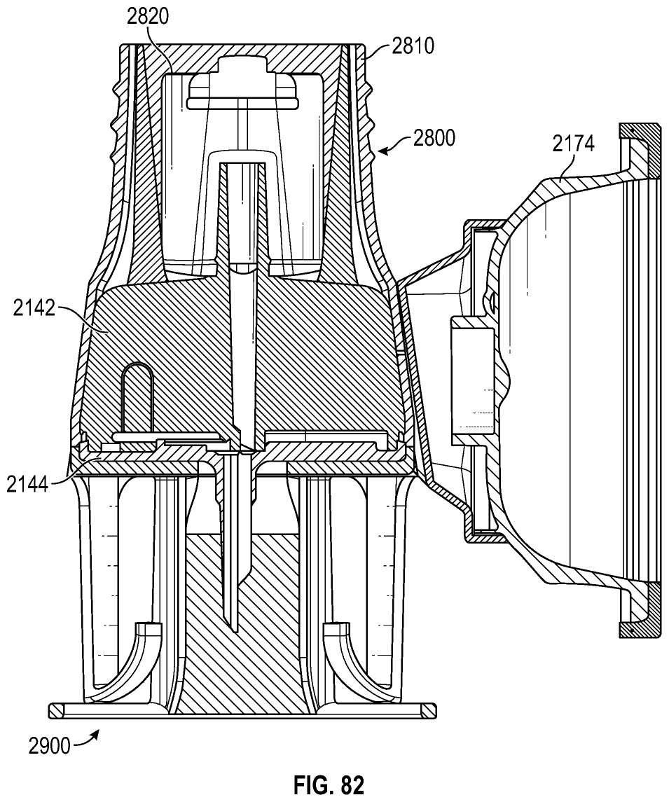

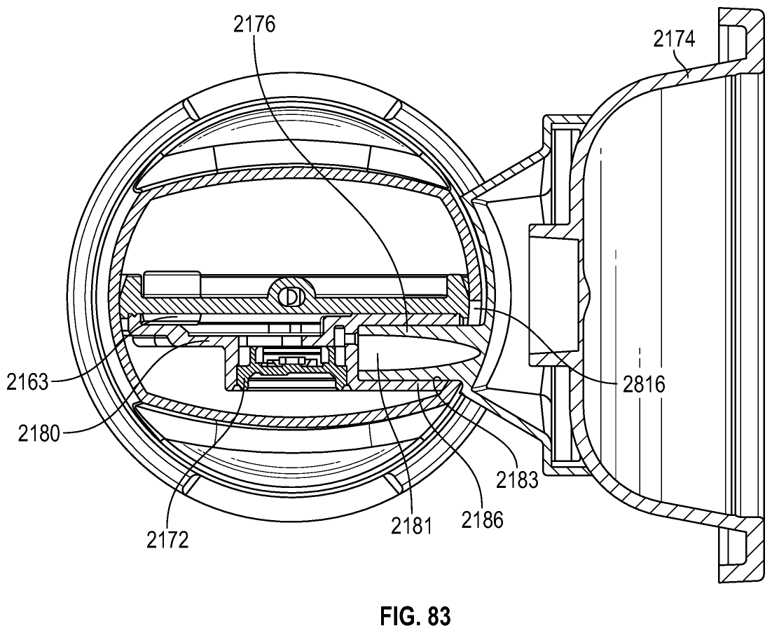

Referring to FIGS. 67-84, another example of a modular CSTD 2100 featuring a vial adaptor is shown. Modular CSTD 2100 is similar to the CSTD's that can be assembled in modular system 2100″ shown in FIGS. 52-61. Many features of modular CSTD 2100 are identical or substantially similar to features of modular system 2100″ and will not be described for brevity.

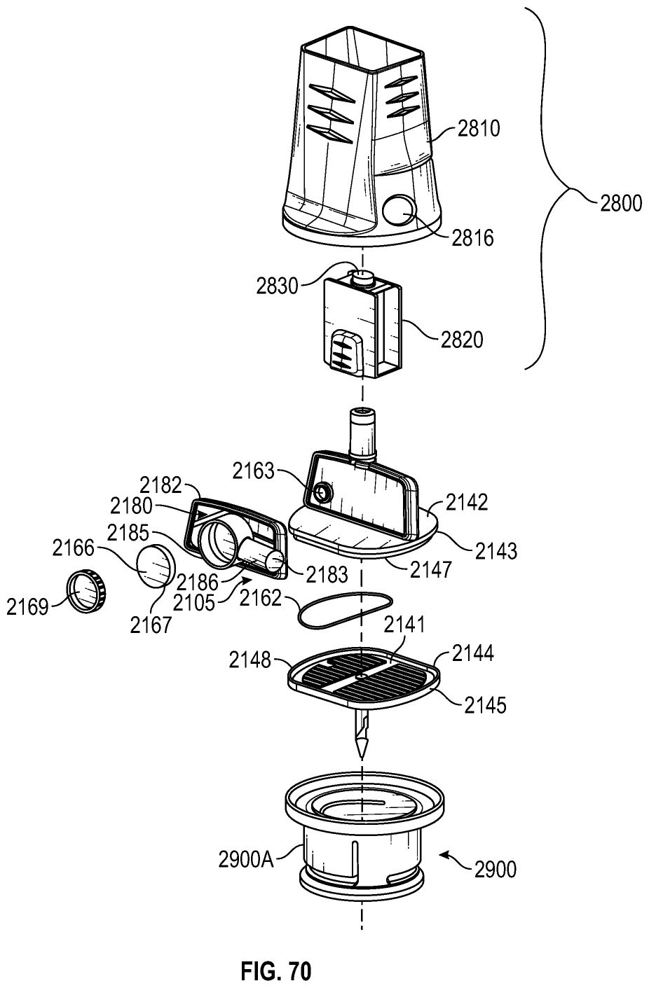

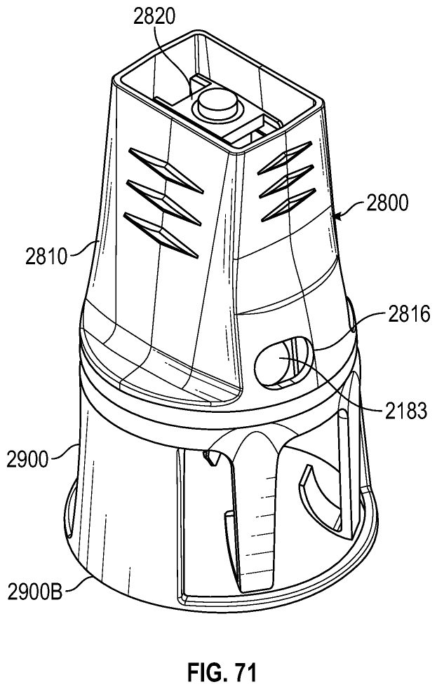

Modular CSTD 2100 includes a core or “base assembly” 2105, as shown in FIG. 70. Base assembly 2105 can be connected to two interchangeable clip modules and two interchangeable venting modules. Unlike the modular CSTD 2100″, the base assembly 2105 in modular CSTD 2100 includes three components: a vial spike 2120, a gas exchange unit 2180, and a modular adapter 2800.

Modular adaptor 2800 includes an external housing 2810 having a first end 2812 and a second end 2814. First end 2812 attaches to vial clip 2900, and second end 2814 is adapted to receive a syringe adaptor. Modular adaptor 2800 also includes an adaptor member 2820 having a first end 2822 and a second end 2824. First end 2822 has a socket 2823 configured to receive a coupling 2260 on vial spike 2120. A septum 2830 is provided in second end 2824 of adaptor member 2820. Second end 2824 and septum 2830 are configured to form a dry break coupling between vial spike 2120 and a syringe adaptor when the latter is inserted into second end 2814 of external housing 2810.

Second end 2814 of external housing 2810 is configured to center a syringe adaptor relative to modular adaptor 2800 as the syringe adaptor enters the second end. The inner geometry of external housing 2810 maintains the septum of the syringe adaptor in alignment with septum 2830 in adaptor member 2820. External housing 2810 therefore serves as a centering mechanism or guide that controls the position of the syringe adaptor during insertion.

Modular adaptor 2800 is configured to work with venting modules that feature either an activated filter housing or an inflatable membrane. Referring to FIG. 70, external housing 2810 has an opening 2816 that accommodates each type of venting module. When CSTD 2100 is equipped with an activated filter housing, opening 2816 serves as both an exhaust port that allows filtered gas to exit the CSTD and a ventilation port that allows air from the atmosphere to enter into the CSTD to equalize pressure. When CSTD 2100 is equipped with an inflatable membrane, opening 2816 provides a passage that allows the inflatable membrane to be plugged into gas exchange unit 2180 inside external housing 2810.

Modular adaptor 2800 can work with components of other vial adaptors, such as the vial spikes shown in FIGS. 37 and 47. In such cases, the septums for forming the dry break couplings are located in the adaptor member 2820 and not in the vial spikes.

Base assembly 2105 forms a central construct around which different CSTDs can be assembled. Base assembly 2105 is configured to connect to one clip module at a time and one venting module at a time to form a CSTD. Therefore, an assembled CSTD can consist of base assembly 2105 connected to one clip module and one venting module.

Base assembly 2105 manages the flow of both liquids and gases through a CSTD after the CSTD is connected to first and second fluid reservoirs. Vial spike 2120 forms a transfer passage that allows liquid in the first fluid reservoir to pass to the second fluid reservoir in a sealed environment. Gas exchange unit 2180 forms part of a vent passage that equalizes pressure within the CSTD after vial spike 2120 is connected to the first and second fluid reservoirs. The transfer passage and vent passage are physically separated from one another.

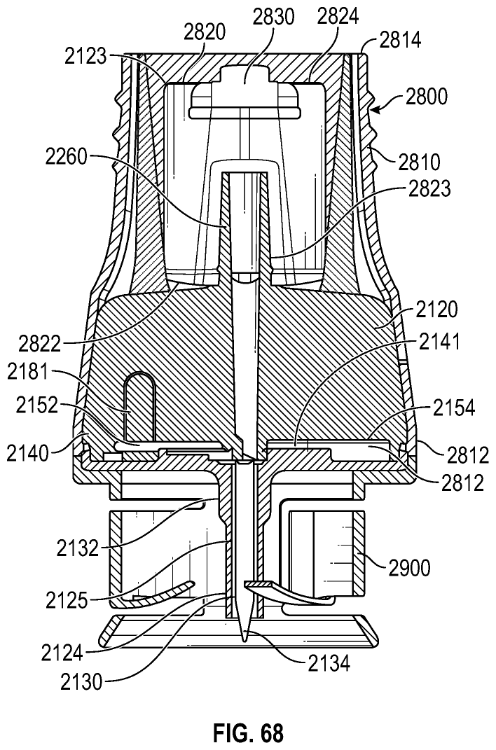

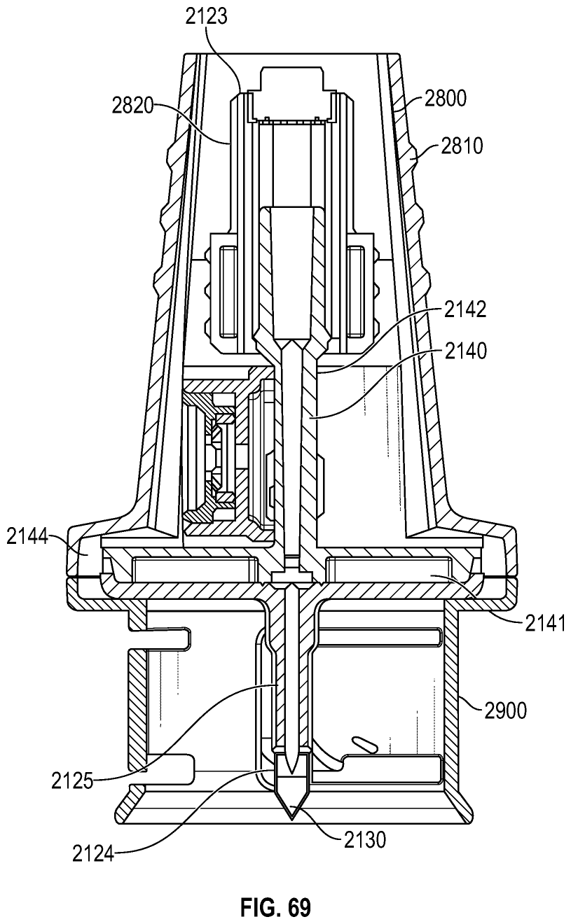

Referring to FIGS. 68 and 69, adaptor member 2820 forms a first connector 2123 configured to fluidly connect to a syringe. A distal end 2124 of vial spike 2120 forms a second connector 2125 configured to fluidly connect to a second fluid reservoir, such as a vial. Once vial spike 2120 is connected in fluid communication with first and second fluid reservoirs, respectively, the vial spike forms a closed fluid passage or transfer line between the first and second fluid reservoirs. The transfer line allows liquid to be transferred between the first and second fluid reservoirs in a sealed manner that prevents release of hazardous drugs into the environment.

Second connector 2125 is a spike connector 2130 having a cylindrical profile 2132 and pointed tip 2134. Spike connector 2130 has separate fluid passages that convey liquid and gas through the CSTD. Vial spike 2120 has a two-part housing 2140 that forms passageways for liquid and gas flowing within a CSTD. Housing 2140 includes a first housing portion 2142 adjacent to and fluidly connected with adaptor member 2820. Housing 2140 also includes a second housing portion 2144 adjacent to and fluidly connected with spike connector 2130.

First housing portion 2142 has a first cover piece 2143, and second housing portion 2144 has a second cover piece 2145. Second cover piece 2145 is configured to connect with first cover piece 2143 to join the first housing portion 2142 and second housing portion 2144 together. First cover piece 2143 and second cover piece 2145 form an enlarged flange section that functions as a finger stop or finger rest, providing more comfort when the device is held.

First cover piece 2143 and second cover piece 2145 also form a narrow gap, void, or space 2141 between first housing portion 2142 and second housing portion 2144. A ring-shaped lip portion 2147 extends around the periphery of first cover piece 2143 and projects from first housing portion 2142. A ring-shaped wall portion 2148 extends around the periphery of second cover piece 2145, forming a receptacle slightly larger in size than the ring-shaped lip portion 2147. Ring-shaped wall portion 2148 is adapted to receive ring-shaped lip portion 2147 to join first housing portion 2142 to second housing portion 2144 in a mated and sealed arrangement.

First cover piece 2143 and second cover piece 2145 create a first chamber 2152 and a second chamber 2154 that are separated by a divider wall 2149 when the first and second cover pieces are mated together. First cover piece 2143 and second cover piece 2145 are connected to one another so that they fully enclose and seal the chambers in a fluid tight manner from the exterior of vial spike 2120. In addition, first cover piece 2143 and second cover piece 2145 are connected to one another to seal the two chambers from one another.

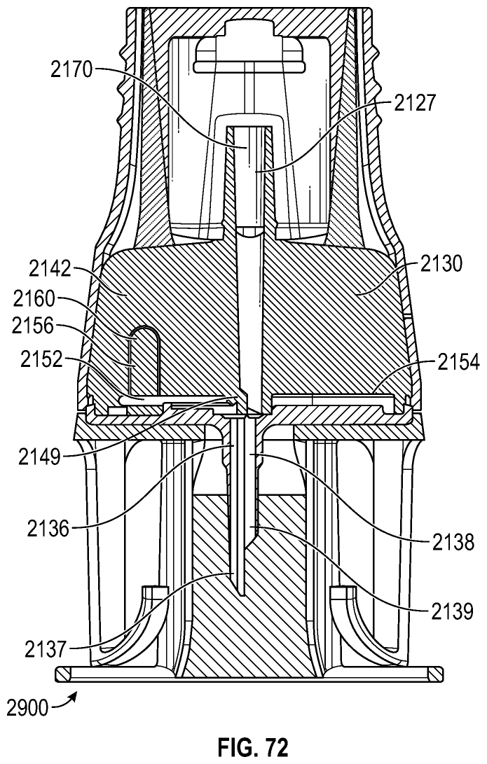

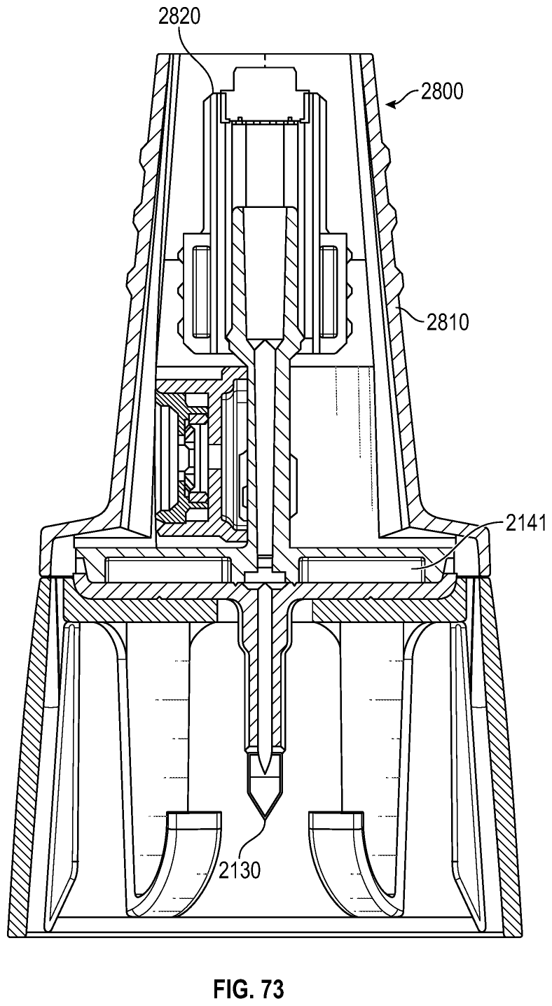

Referring to FIG. 72, spike connector 2130 has a first passage 2136 and a second passage 2138 extending parallel to the first passage. When vial spike 2120 is fully assembled, first passage 2136 fluidly connects with first chamber 2152 but not with second chamber 2154. In addition, second passage 2138 fluidly connects to second chamber 2154 but not first chamber 2152. First passage 2136 extends the entire length of spike connector 2130 and exits pointed tip 2134 where it forms a first opening 2137. Second fluid passage 2138 also extends the entire length of spike connector 2130 and exits pointed tip 2134 where it forms a second opening 2139. First chamber 2152 fluidly connects to a vent passage 2156 in first housing portion 2142. Second chamber 2154 fluidly connects to a fluid passage 2127 in coupling 2260. In this arrangement, first passage 2136 forms part of a vent line 2160 that equalizes pressure in modular CSTD 2100 with outside atmospheric pressure, while second passage 2138 forms part of a transfer line 2170 for transferring liquid between the first fluid reservoir and second fluid reservoir. Vent line 2160 and transfer line 2170 are sealed from one another within the CSTD, so that gas carried in vent line 2160 does not enter transfer line 2170, and liquid carried in the transfer line does not enter the vent line.

Each chamber in modular CSTD 2100 is configured to contain a filter material or filter component dedicated to each respective line. FIGS. 70, 74, 79 and 84 each show only a hydrophobic filter component 2162 for placement in vent line 2160. It will be appreciated that modular CSTD 2100 can also include a particle filter in transfer line 2170.

Vent line 2160 allows air from the atmosphere to enter the CSTD. Hydrophobic filter 2162 is configured to allow air from the atmosphere to pass through the hydrophobic filter, while not allowing liquids and aerosols in the air to pass through the hydrophobic filter. This prevents contaminants from the atmosphere from passing through the first chamber 2152, through the first passage 2136 and into the second fluid reservoir. It also prevents or substantially prevents liquids and aerosols from the second fluid reservoir from going beyond the first chamber 2152. Hydrophobic filter 2162 can have any suitable pore size, including but not limited to a pore size of 0.2 microns.

Vent line 2160 extends from first passage 2136, through first chamber 2152 and into first housing portion 2142. Vent line 2160 exits first housing portion 2142 through a side port 2163 and into gas exchange unit 2180.

Gas exchange unit 2180 has a hollow shell structure or body 2182 that connects to one side of first housing portion 2142 and over side port 2163. The interior of body 2182 provides a gas exchange passage 2181 that forms a section of vent line 2160. The gas exchange passage 2181 exits gas exchange unit 2180 through an elongated socket 2186. Gas in socket 2186 exits body 2182 through a port 2183 to the exterior of the body.

Gas exchange unit 2180 is configured to connect to different venting modules as noted above. In particular, gas exchange unit 2180 can connect to a filter unit that filters hazardous gas from the CSTD before discharging the gas to the atmosphere. Alternatively, gas exchange unit 2180 can connect to a gas storage unit that retains hazardous gas inside the CSTD and prevents the gas from being released to the atmosphere. This versatility allows different CSTDs to be manufactured and assembled using one base assembly design, increasing manufacturing efficiency, as noted earlier.

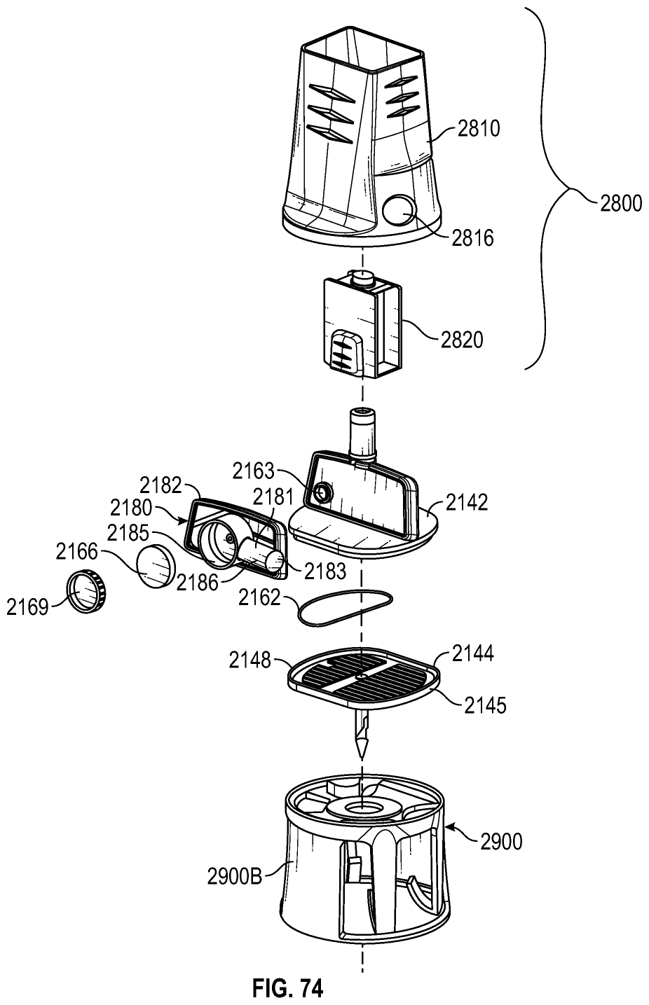

FIGS. 67-74 show modular assemblies that combine base assembly 2105 with a filter unit. Referring to FIGS. 70 and 74, the filter unit includes an activated carbon filter 2166 designed to filter hazardous gas in vent line 2160. A plug-in receptacle 2185 forms a filter housing adapted to receive activated carbon filter 2166. A cap 2169 closes plug-in receptacle 2185 in an airtight arrangement after activated carbon filter 2166 is received in the filter housing to retain and protect the activated carbon filter inside gas exchange unit 2180. During operation, hazardous gas in vent line 2160 passes into gas exchange passage 2181 and through activated carbon filter 2166 where it is filtered to remove toxins. The filtered gas then exits the filter housing and passes through elongated socket 2186 to port 2183. From there, the gas flows through opening 2816 in external housing 2810 and discharges to the atmosphere.

The gas exchange passage 2181 provides a two-way ventilation route for vent line 2160 when a filter unit is used with gas exchange unit 2180. That is, gas can flow through gas exchange passage 2181 in a first direction, where the gas is filtered through activated carbon filter 2166 and released to the atmosphere, as described above. In addition, gas exchange passage 2180 allows air from the atmosphere to enter through second port 2182 and flow through vent line 2160 in a second direction opposite the first direction to equalize pressure in the CSTD.

Activated carbon filters according to the present disclosure can have various geometries. In the present example, activated carbon filter 2166 is a cylindrical-shaped filter medium. Gas flows through a sidewall 2167 of activated carbon filter 2166 in a “lateral” direction. This provides a longer flow path and ensures that the entire volume of the filter is utilized to filter hazardous components, allowing more filtering to take place and increasing filter capacity.

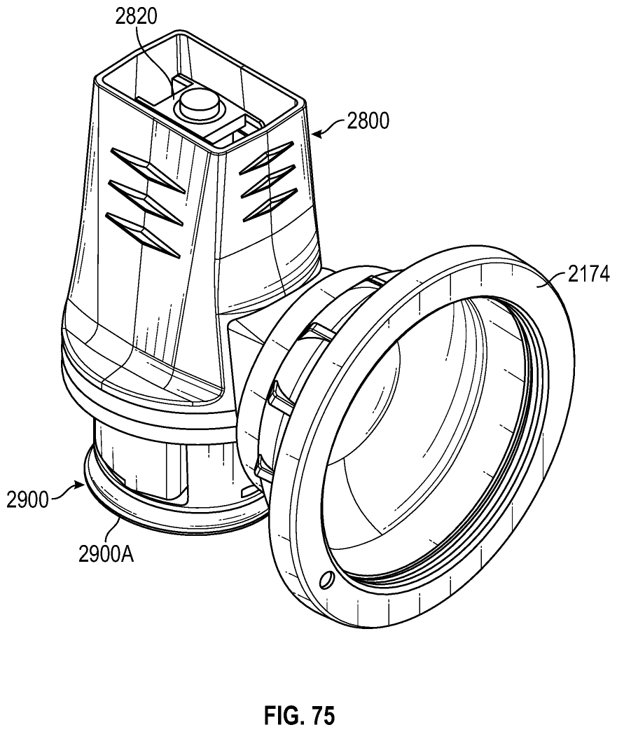

FIGS. 75-84 show modular assemblies that combine base assembly 2105 with an inflatable reservoir 2174. The membrane portion of inflatable reservoir 2174 is omitted in the drawing figures for clarity, with the understanding that membranes such as those appearing in FIGS. 47 and 52 can be attached.

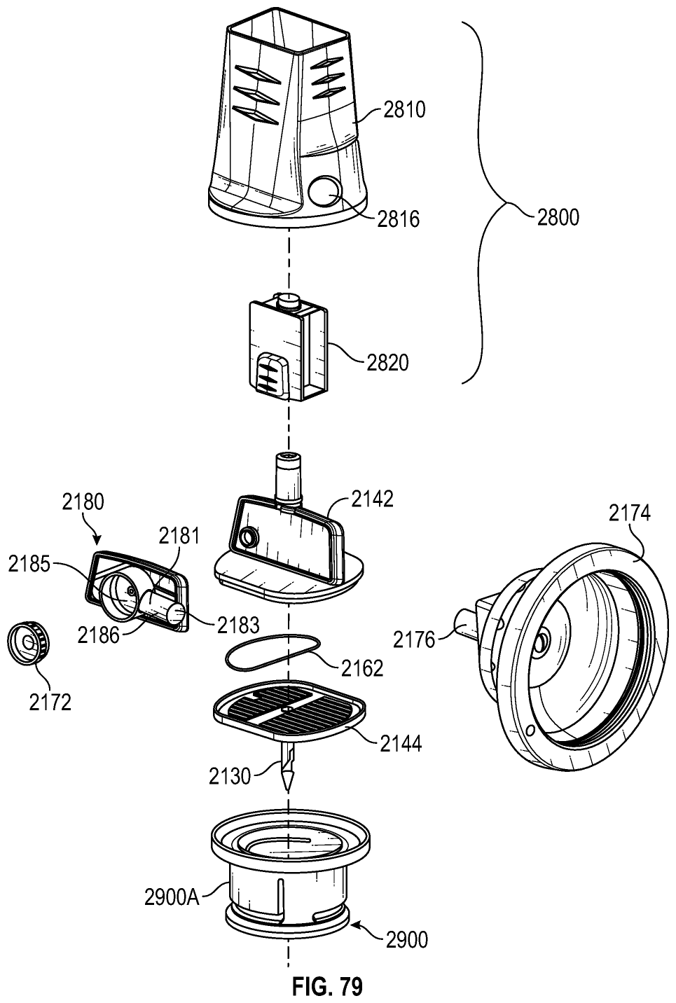

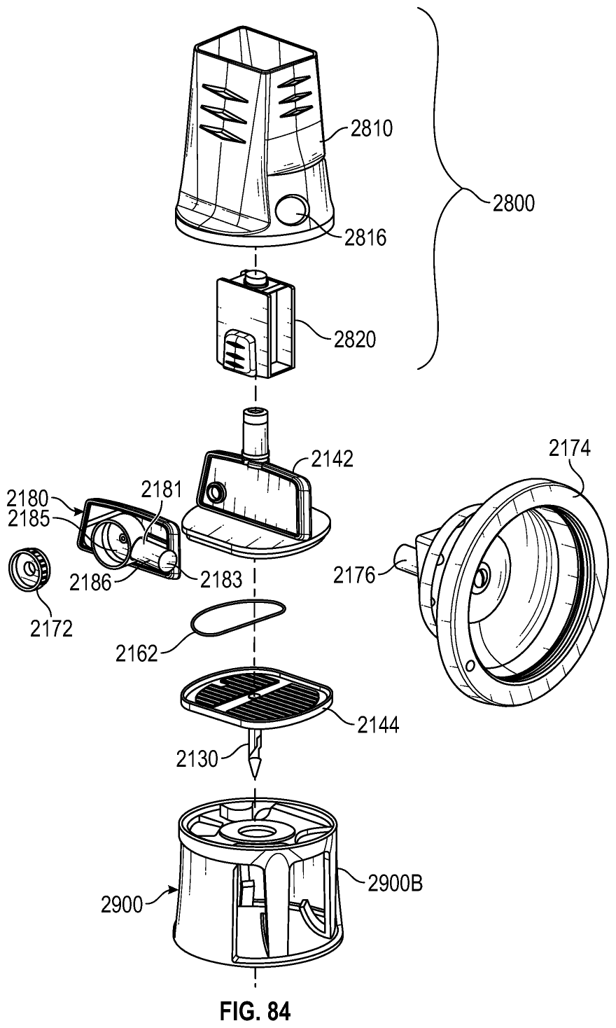

Referring to FIGS. 79 and 84, the venting module includes a check valve 2172. Plug-in receptacle 2185 is configured to connect to and receive check valve 2172. Check valve 2172 is a one-way valve that allows air from the atmosphere to enter gas exchange unit 2180 but prevents gas from exiting the gas exchange unit. Check valve 2172 is thus configured to open to allow air from the atmosphere to enter gas exchange unit 2180 and flow into vent line 2160 to equalize pressure in the CSTD. Check valve 2172 is also configured to close in response to positive pressure in the gas exchange passage 2181, such as when hazardous gas is released from a reservoir into the gas exchange passage, preventing the hazardous gas from escaping to the atmosphere through the check valve.

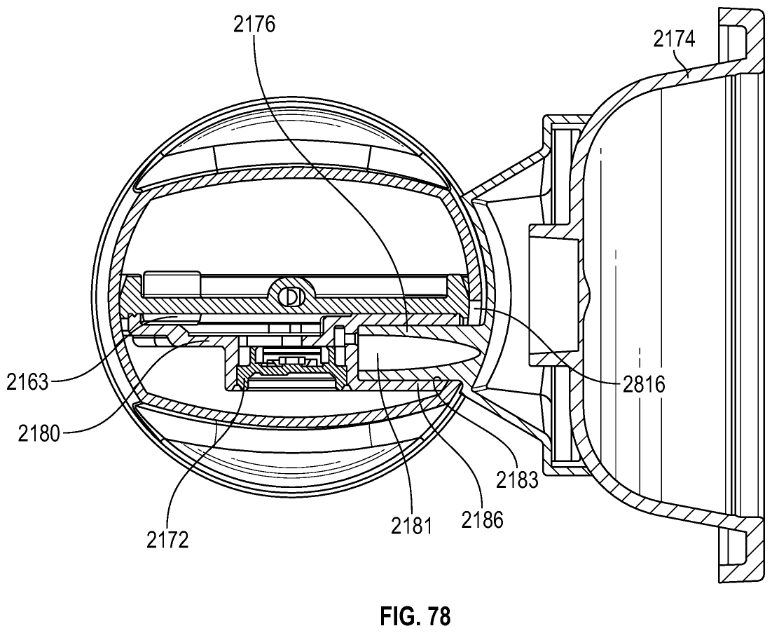

Reservoir 2174 is configured to connect to port 2183 on gas exchange unit 2180 to form an expandable balloon for the storage of gas. Referring to FIGS. 78, 79, 83 and 84, reservoir 2174 has a hollow tubular stem 2176 defining a passage into and out of the reservoir. Tubular stem 2176 can be inserted through opening 2816 in external housing 2810 and plugged into socket 2186 to connect reservoir 2174 to gas exchange unit 2180. In this arrangement, reservoir 2174 is configured to collect gas that enters gas exchange unit 2180 from vent line 2160. Reservoir 2174 is also configured to discharge stored gas into the gas exchange passage 2181 and vent line 2160 to balance pressure when pressure drops in the CSTD. When reservoir 2174 collects gas, the membrane expands, providing a visible indicator or alert that hazardous gas is being released from one of the reservoirs and stored inside the CSTD. When reservoir 2174 discharges gas, the membrane collapses, creating a visible indicator or alert that pressure is being equalized in the CSTD.

When gas is released from a vial after being penetrated with spike connector 2130, the gas will travel through vial spike 2120 and into gas exchange unit 2180. Once inside gas exchange unit 2180, the gas flows through plug-in receptacle 2185 and is trapped and collected by reservoir 2174. The trapped gas can remain stored in reservoir 2174 until a pressure drop occurs in the CSTD. When a pressure drop occurs in the CSTD, gas stored in reservoir 2174 is released into gas exchange passage 2181 and vent line 2160 to balance pressure. If additional gas is needed to balance pressure, check valve 2172 is configured to allow air outside CSTD 2100 to flow into first housing portion 2142 to balance the pressure.

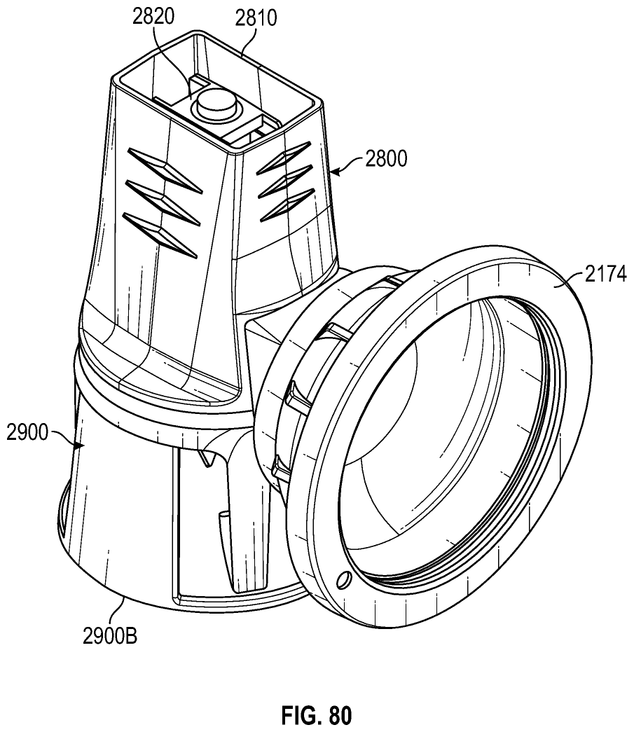

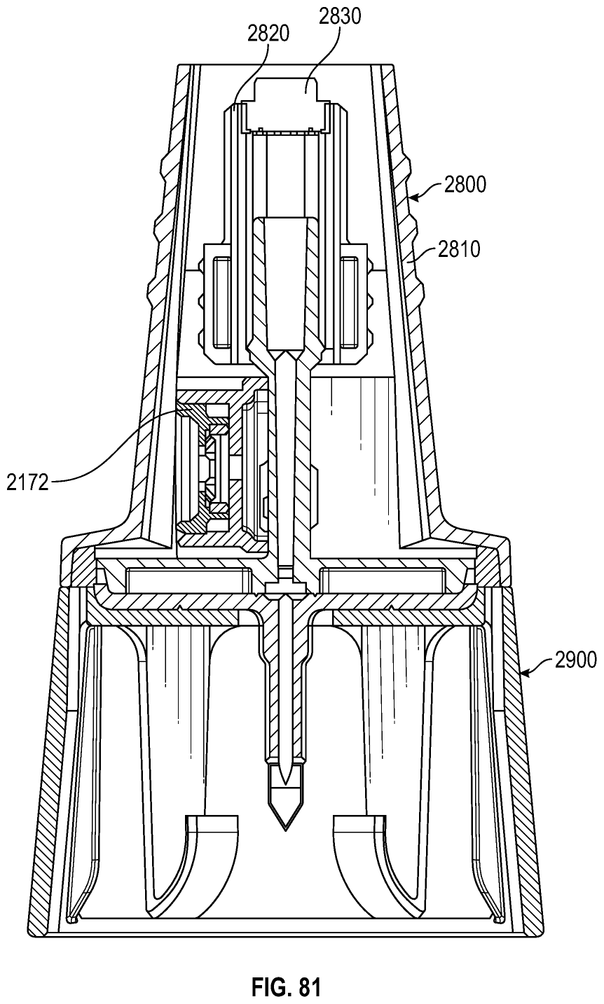

FIGS. 67, 71, 75 and 80 show four different CSTDs that can be assembled using different modules in modular system 2100. The CSTD's in FIGS. 67 and 75 have vial clips 2900A designed to connect to 13-20 mm vials. The CSTD's in FIGS. 71 and 80 have vial clips 2900B designed to connect to 32 mm vials. It will be appreciated that modular systems according to the present disclosure can include other clip modules designed to connect to any vial size or range of sizes, as well as other types of containers.

1. A closed system transfer device comprising:
a first adaptor configured to attach to a first reservoir, the first adaptor defining a first passage and comprising a first septum that seals an end of the first passage;
a second adaptor configured to attach to a second reservoir; the second adaptor comprising a housing having an interior, the housing defining a second passage and comprising a second septum that seals an end of the second passage;
a carrier movable in the interior of the second adaptor, the carrier defining a chamber that contains at least a portion of the second septum; and
a needle disposed in the interior of the second adaptor, the needle having a needle opening,
the interior of the second adaptor adapted to receive the first adaptor in a telescoping manner, with the first adaptor insertable into the second adaptor,
the carrier displaceable in the interior of the second adaptor, and relative to the needle, by the first adaptor when the first adaptor is inserted into the second adaptor,
the carrier displaceable within the second adaptor between a first position, in which the first septum abuts the second septum and the needle opening is sealed inside the second passage, and a second position, in which the first septum abuts the second septum and the needle opening is in fluid communication with the first passage to connect the first adaptor and the second adaptor in a fluid path open state.

2. The closed system transfer device according to claim 1 comprising a releasable lock that locks the first adaptor inside the second adaptor after the first adaptor is inserted into the second adaptor.

3. The closed system transfer device according to claim 2, wherein the releasable lock locks the first adaptor inside the second adaptor when the carrier is displaced to the second position.

4. The closed system transfer device according to claim 2, wherein the releasable lock comprises a first locking element on the carrier and a second locking element in the housing.

5. The closed system transfer device according to claim 2, wherein the releasable lock comprises a first locking element on the first adaptor and a second locking element on the second adaptor.

6. The closed system transfer device according to claim 2, wherein the releasable lock is released by pressing at least one side of the housing radially inwardly.

7. The closed system transfer device according to claim 6, wherein the at least one side of the housing comprises at least one push button.

8. The closed system transfer device according to claim 7, wherein the at least one push button is depressible radially inwardly to disengage a portion of the carrier from a section of the housing.

9.-12. (canceled)

13. The closed system transfer device according to claim 1, wherein the second adaptor comprises a third septum that is axially spaced from the second septum.

14. The closed system transfer device according to claim 13, wherein the needle opening is sealed between the second septum and the third septum when the carrier is in the first position.

15. (canceled)

16. (canceled)

17. The closed system transfer device according to claim 1 further comprising a female Luer connector connected to the housing of the second adaptor.

18. (canceled)

19. The closed system transfer device according to claim 1, wherein the housing defines at least one locking window configured to retain the carrier in one of the first position and the second position with at least a portion of the carrier visible through the at least one locking window to visually confirm that the carrier is in said one of the first position and the second position.

20. The closed system transfer device according to claim 1, wherein the first adaptor comprises a male Luer connector or a vial spike.

21. The closed system transfer device according to claim 1, wherein the first adaptor and the second adaptor are rectangular.

22. (canceled)

23. The closed system transfer device according to claim 17, wherein the needle is fixed in an interior of the female Luer connector.

24. The closed system transfer device according to claim 2, wherein the releasable lock comprises a first locking element on the carrier and a second locking element on the first adaptor.

25. The closed system transfer device according to claim 24, wherein the releasable lock is released by pressing at least one side of the housing radially inwardly.

26. The closed system transfer device according to claim 25, wherein the at least one side of the housing comprises a push button.

27.-100. (canceled)

101. The closed system transfer device according to claim 19, wherein the at least one locking window comprises a pair of first locking windows that extend through the housing, the first locking windows configured to lockingly engage a pair of lugs on the carrier to retain the carrier in the first position.

102. The closed system transfer device according to claim 101, wherein the at least one locking window further comprises a pair of second locking windows that extend through the housing, the second locking windows configured to lockingly engage the pair of lugs on the carrier to retain the carrier in the second position.

103. The closed system transfer device according to claim 102, wherein the pair of lugs are visible through the first locking windows and the second locking windows from outside the closed system transfer device, thereby providing a visible indicator of a position of the carrier and an operating state of the closed system transfer device.
